SEQUENCE LISTING (39 sequences)

SEQ ID NO: 1 - DNA, 39 bp, Artificial
  Primer 1
attattcgcgatggctcctggcatctctgggaccgaggc

SEQ ID NO: 2 - DNA, 39 bp, Artificial
  Primer 2
atcgtcgcgaagcttgagattgtccaagcaggtagccag

SEQ ID NO: 3 - DNA, 36 bp, Artificial
  Primer 3
agcaagtacttctgagccttcagtaatggctgcctg

SEQ ID NO: 4 - DNA, 39 bp, Artificial
  Primer 4
tggcagtactaagctggcaccactacttcagagaacaag

SEQ ID NO: 5 - DNA, 3179 bp, Artificial
  5′ GAPDH fragment
atcgtcgcgaagcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccatcgcgaataat

SEQ ID NO: 6 - DNA, 3238 bp, Artificial
  3′-GAPDH fragment
tggcagtactaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagttttgaggaagatgaactgagatgtgtcagggtgacttatttccatcatcgtccttaggggaacttgggtaggggcaaggcgtgtagctgggacctaggtccagacccctggctctgccactgaacggctcagttgctttgggcagttactcccgggcctcactttgcacgtgtgcttacctagtggagacaaaagtacatacctcggtagagcgcgcacgcctgtaaccccagcactttgggaggccaaggtgggtgtatcacctgaggtcaggagtttgagaccagcctggccaacatggtgaaactccgtctctactaaaattacaaaaatcagccaggcttcatggcacatgcctatagtcccagctacaggcatgctgaagcaggagaatcgcttgcaccccggaggcagaggctgcagtgagctgagaccacaccactgcactccagcctaggcaacagagtatgagactccatctcaaaaaaaaaaaaagtacctacctcagagttcaaactagtgaatattaggaagtgcttgagacagtgacaccaaagtgcacaataaatactcgccagtttcattattattaaagaatccatttgaatgtcagctcaacacagcctcctataccgaggcattgtgaaccgcatctccccagcttctccaggcttttccaagaatcagggacactgtagcctgttggtctcagtgtatgacagacacggaggaagcacatctttagctgatacttaaacagagaccctgagcgcacatacacccgcgcacacatgcatggagcttcaccttctctgtcattctgcagtgaccaggagagcaagagctcccacctcccttcaaaacactgtgcccatcccgggcactaaggcctctttaaagcacggcacctccacgagggagggccacagccacatacactccacctggcaggtggacagcgtgagcacgtggaccatagcagggacaaggtgccccggccagccccaacgccctctgccgctgacagggacagaagccctctccagctgcgtgtgctgcagaggccatgcgtagcctccagctgcattctattccactccagtgcctgggccagttagcaccagtgtggaagacagtgagctggctccggacaacagggatggaggaaaggtcccacattcacattcctgatacgtggacaaggtgaggggccgcaatcgctctggcagcattttaaagatggggaagtagcagacacccacgcgtgaaggcaggagagccccaactgtggtggaaatggccccagaatggtagggccaagcctagctccagacaccccagagccctggagaagccaagactgagggagaaagcctgagggaggagcgccccagtccccagggaccggcctggtgcagagctgcagctgatgttcccctctgtgcagccccaccctctgcctcgctgagctccctgctgcgagggcctcgggtgcaagggggaggcaggtctctatctcatggagctgtcagatgagacatcgcgatcggagtcctcagcctcgcttggcggcggcggcgggtcgctaagcgggaccgcagtgaaagcaggagactttctagaaaaaaacaccagttgtcaaccttggggcaggcaggaatcctgaagacggacggcactcctcctcctgctgcctcaccctctggcagcccgtgagaagtaccggaagcgagggcggggccgcgggatggcgagggagcggcagggactgaactctctccaaacccaccctgacagggaaatgggccccgcctgtgtcttgggaactcagaggctgaggtcaggcatgatggctcacgcctgtaattccagcactttgggaggcagaggcgggtggatcacgacgtcaggagttcaaggcaagcctggccaacatggtgaaaccccatctctactaaaaatgcaaaaattagccaggtgtggtggcgggcgccttggaggctgaggcaagataatcacttgaacctgggaggcggaggttgtagtgagccaaaaaaaaaaaaaaaagaaatagctgaagtcacagtaggagagaagctgctgagcctccagcaccctgactctagggccttggctttatgtctatctgcagtatttttgtgatttttaaaaattcactttcttgttgcggtgtaacttacacagggtcaaatgcacaaatcatggccctgactttagataaaaatctgcccccacaacccttctgttccttgccagtttttaaactgcctctaaccaggggaaccaccagagctggtggccttgggaggtttcagccctcccgtcatgaatggacatagctcatccaactgccaagggagagagctgtgggtctgggccagccccaccaggtaactcccaaagggcagccccacagcaagatgtgacccagtcattgcctgagggtctctggggctgtgttccaacctttctccccgctgtgtccccctggaaggccccatgcccaggggaggcgcctaccggtctccagactgggtgatgagccggcggcaggtctccatctgcacgtccaggccgcgcttcatgctgcacatctccatgtactcgtgcaggtgccggttcatgtcgttcttggccgtggccaactccagctgtggtggggcaggcagggccatcggggttaacaggtggcgttcacagcgcctctgttgcccccgccaggaggccaacacgccaagagcagtggctgggccgggggcccaggcagccattactgaaggctcagaagtacttgct

SEQ ID NO: 7 - DNA, 3164 bp, Artificial
  Upstream GAPDH flanking region
cgaagcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccatcg

SEQ ID NO: 8 - DNA, 3224 bp, Artificial
  Downstream GAPDH flanking region
actaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagttttgaggaagatgaactgagatgtgtcagggtgacttatttccatcatcgtccttaggggaacttgggtaggggcaaggcgtgtagctgggacctaggtccagacccctggctctgccactgaacggctcagttgctttgggcagttactcccgggcctcactttgcacgtgtgcttacctagtggagacaaaagtacatacctcggtagagcgcgcacgcctgtaaccccagcactttgggaggccaaggtgggtgtatcacctgaggtcaggagtttgagaccagcctggccaacatggtgaaactccgtctctactaaaattacaaaaatcagccaggcttcatggcacatgcctatagtcccagctacaggcatgctgaagcaggagaatcgcttgcaccccggaggcagaggctgcagtgagctgagaccacaccactgcactccagcctaggcaacagagtatgagactccatctcaaaaaaaaaaaaagtacctacctcagagttcaaactagtgaatattaggaagtgcttgagacagtgacaccaaagtgcacaataaatactcgccagtttcattattattaaagaatccatttgaatgtcagctcaacacagcctcctataccgaggcattgtgaaccgcatctccccagcttctccaggcttttccaagaatcagggacactgtagcctgttggtctcagtgtatgacagacacggaggaagcacatctttagctgatacttaaacagagaccctgagcgcacatacacccgcgcacacatgcatggagcttcaccttctctgtcattctgcagtgaccaggagagcaagagctcccacctcccttcaaaacactgtgcccatcccgggcactaaggcctctttaaagcacggcacctccacgagggagggccacagccacatacactccacctggcaggtggacagcgtgagcacgtggaccatagcagggacaaggtgccccggccagccccaacgccctctgccgctgacagggacagaagccctctccagctgcgtgtgctgcagaggccatgcgtagcctccagctgcattctattccactccagtgcctgggccagttagcaccagtgtggaagacagtgagctggctccggacaacagggatggaggaaaggtcccacattcacattcctgatacgtggacaaggtgaggggccgcaatcgctctggcagcattttaaagatggggaagtagcagacacccacgcgtgaaggcaggagagccccaactgtggtggaaatggccccagaatggtagggccaagcctagctccagacaccccagagccctggagaagccaagactgagggagaaagcctgagggaggagcgccccagtccccagggaccggcctggtgcagagctgcagctgatgttcccctctgtgcagccccaccctctgcctcgctgagctccctgctgcgagggcctcgggtgcaagggggaggcaggtctctatctcatggagctgtcagatgagacatcgcgatcggagtcctcagcctcgcttggcggcggcggcgggtcgctaagcgggaccgcagtgaaagcaggagactttctagaaaaaaacaccagttgtcaaccttggggcaggcaggaatcctgaagacggacggcactcctcctcctgctgcctcaccctctggcagcccgtgagaagtaccggaagcgagggcggggccgcgggatggcgagggagcggcagggactgaactctctccaaacccaccctgacagggaaatgggccccgcctgtgtcttgggaactcagaggctgaggtcaggcatgatggctcacgcctgtaattccagcactttgggaggcagaggcgggtggatcacgacgtcaggagttcaaggcaagcctggccaacatggtgaaaccccatctctactaaaaatgcaaaaattagccaggtgtggtggcgggcgccttggaggctgaggcaagataatcacttgaacctgggaggcggaggttgtagtgagccaaaaaaaaaaaaaaaagaaatagctgaagtcacagtaggagagaagctgctgagcctccagcaccctgactctagggccttggctttatgtctatctgcagtatttttgtgatttttaaaaattcactttcttgttgcggtgtaacttacacagggtcaaatgcacaaatcatggccctgactttagataaaaatctgcccccacaacccttctgttccttgccagtttttaaactgcctctaaccaggggaaccaccagagctggtggccttgggaggtttcagccctcccgtcatgaatggacatagctcatccaactgccaagggagagagctgtgggtctgggccagccccaccaggtaactcccaaagggcagccccacagcaagatgtgacccagtcattgcctgagggtctctggggctgtgttccaacctttctccccgctgtgtccccctggaaggccccatgcccaggggaggcgcctaccggtctccagactgggtgatgagccggcggcaggtctccatctgcacgtccaggccgcgcttcatgctgcacatctccatgtactcgtgcaggtgccggttcatgtcgttcttggccgtggccaactccagctgtggtggggcaggcagggccatcggggttaacaggtggcgttcacagcgcctctgttgcccccgccaggaggccaacacgccaagagcagtggctgggccgggggcccaggcagccattactgaaggctcagaagt

SEQ ID NO: 9 - DNA, 511 bp, Artificial
  Fragment 1
cgaagcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgat

SEQ ID NO: 10 - DNA, 2653 bp, Artificial
  Fragment 2
atctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccatcg

SEQ ID NO: 11 - DNA, 1966 bp, Artificial
  Fragment 3
cgaagcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacac

SEQ ID NO: 12 - DNA, 1198 bp, Artificial
  Fragment 4
tggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccatcg

SEQ ID NO: 13 - DNA, 259 bp, Artificial
  Fragment 8
cctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccatcg

SEQ ID NO: 14 - DNA, 1947 bp, Artificial
  Fragment 9
cgaagcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccatcg

SEQ ID NO: 15 - DNA, 1436 bp, Artificial
  Fragment 11
atctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccatcg

SEQ ID NO: 16 - DNA, 1177 bp, Artificial
  Fragment 17
atctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggagg

SEQ ID NO: 17 - DNA, 18106 bp, Homo sapiens
gtgagcagcacaggacacttcaatgcctgttgggttctgggctggctaagacatctgccggccctgggcagcatacggctcttgcagtcaccttcccgtcctccttatccccagctgggttgcaaccaaattgccagagtgacctaagaccagatctttgtctccagttctttttttattactccaaaaacacaaccaaagcagcatctcatccaattcttgtttgtttgtttttaatagtttttatttttcagagcagttttaggttcaaagcaaaattgagcagaaagtacagggagttcccttctaccccttgcccctacacatcacagccttccccaccttcaacatcctgcaccagggtggcacatttgttacagctgaacctacacttacacatcatctcctaaagtcatggtttaccttggagttcactgcacgtaatgacatgtacccaccattgcagtatcatacagaagagtttcactgccttacaaatcccctgcactccacctatttatccctctctccccacaacccctgatctttttactgttgccatcactttgtcttttccagaatgtatcattggaatgatccggtatggagccttctcaccttggcttcttagtaatgtgcgtttaaggcctccatgtcttccatggccttgtttctttttaatcagaagtaactgttttcaggcctgctctgaatctccttttctccctccaggctataatagatgaatttgagcagaagcttcgggcctgtcataccagaggtttggatggaatcaaggagcttgagattggccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccaggagatggggagggggaagtgggggctgggaaggaaccacgggcccccgcccgaggcccatgggcccctcctaggcctttgcctgagcagtccggtgtcactaccgcagagcctcgaggagaagttccccaactttcccgcctctcagcctttgaaagaaagaaaggggagggggcaggccgcgtgcagccgcgagcggtgctgggctccggctccaattccccatctcagtcgttcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgtgtgtcggccggggccactaggcgctcactgttctctccctccgcgcagccgagccacatcgctcagacaccatggggaaggtgaaggtcggagtcaacgggtgagttcgcgggtggctggggggccctgggctgcgaccgcccccgaaccgcgtctacgagccttgcgggctccgggtctttgcagtcgtatgggggcagggtagctgttccccgcaaggagagctcaaggtcagcgctcggacctggcggagccccgcacccaggctgtggcgccctgtgcagctccgcccttgcggcgccatctgcccggagcctccttcccctagtccccagaaacaggaggtccctactcccgcccgagatcccgacccggacccctaggtgggggacgctttctttcctttcgcgctctgcggggtcacgtgtcgcagaggagcccctcccccacggcctccggcaccgcaggccccgggatgctagtgcgcagcgggtgcatccctgtccggatgctgcgcctgcggtagagcggccgccatgttgcaaccgggaaggaaatgaatgggcagccgttaggaaagcctgccggtgactaaccctgcgctcctgcctcgatgggtggagtcgcgtgtggcggggaagtcaggtggagcgaggctagctggcccgatttctcctccgggtgatgcttttcctagattattctctggtaaatcaaagaagtgggtttatggaggtcctcttgtgtcccctccccgcagaggtgtggtggctgtggcatggtgccaagccgggagaagctgagtcatgggtagttggaaaaggacatttccaccgcaaaatggcccctctggtggtggccccttcctgcagcgccggctcacctcacggccccgcccttcccctgccagcctagcgttgacccgaccccaaaggccaggctgtaaatgtcaccgggaggattgggtgtctgggcgcctcggggaacctgcccttctccccattccgtcttccggaaaccagatctcccaccgcaccctggtctgaggttaaatatagctgctgacctttctgtagctgggggcctgggctggggctctctcccatcccttctccccacacacatgcacttacctgtgctcccactcctgatttctggaaaagagctaggaaggacaggcaacttggcaaatcaaagccctgggactagggggttaaaatacagcttcccctcttcccacccgccccagtctctgtcccttttgtaggagggacttagagaaggggtgggcttgccctgtccagttaatttctgacctttactcctgccctttgagtttgatgatgctgagtgtacaagcgttttctccctaaagggtgcagctgagctaggcagcagcaagcattcctggggtggcatagtggggtggtgaataccatgtacaaagcttgtgcccagactgtgggtggcagtgccccacatggccgcttctcctggaagggcttcgtatgactgggggtgttgggcagccctggagccttcagttgcagccatgccttaagccaggccagcctggcagggaagctcaagggagataaaattcaacctcttgggccctcctgggggtaaggagatgctgcattcgccctcttaatggggaggtggcctagggctgctcacatattctggaggagcctcccctcctcatgccttcttgcctcttgtctcttagatttggtcgtattgggcgcctggtcaccagggctgcttttaactctggtaaagtggatattgttgccatcaatgaccccttcattgacctcaactacatggtgagtgctacatggtgagccccaaagctggtgtgggaggagccacctggctgatgggcagccccttcataccctcacgtattcccccaggtttacatgttccaatatgattccacccatggcaaattccatggcaccgtcaaggctgagaacgggaagcttgtcatcaatggaaatcccatcaccatcttccaggagtgagtggaagacagaatggaagaaatgtgctttggggaggcaactaggatggtgtggctcccttgggtatatggtaaccttgtgtccctcaatatggtcctgtccccatctcccccccacccccataggcgagatccctccaaaatcaagtggggcgatgctggcgctgagtacgtcgtggagtccactggcgtcttcaccaccatggagaaggctggggtgagtgcaggagggcccgcgggaggggaagctgactcagccctgcaaaggcaggacccgggttcataactgtctgcttctctgctgtaggctcatttgcaggggggagccaaaagggtcatcatctctgccccctctgctgatgcccccatgttcgtcatgggtgtgaaccatgagaagtatgacaacagcctcaagatcatcaggtgaggaaggcagggcccgtggagaagcggccagcctggcaccctatggacacgctcccctgacttgcgccccgctccctctttctttgcagcaatgcctcctgcaccaccaactgcttagcacccctggccaaggtcatccatgacaactttggtatcgtggaaggactcatggtatgagagctggggaatgggactgaggctcccacctttctcatccaagactggctcctccctgccggggctgcgtgcaaccctggggttgggggttctggggactggctttcccataatttcctttcaaggtggggagggaggtagaggggtgatgtggggagtacgctgcagggcctcactccttttgcagaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactcttttcatcttctaggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagttttgaggaagatgaactgagatgtgtcagggtgacttatttccatcatcgtccttaggggaacttgggtaggggcaaggcgtgtagctgggacctaggtccagacccctggctctgccactgaacggctcagttgctttgggcagttactcccgggcctcactttgcacgtgtgcttacctagtggagacaaaagtacatacctcggtagagcgcgcacgcctgtaaccccagcactttgggaggccaaggtgggtgtatcacctgaggtcaggagtttgagaccagcctggccaacatggtgaaactccgtctctactaaaattacaaaaatcagccaggcttcatggcacatgcctatagtcccagctacaggcatgctgaagcaggagaatcgcttgcaccccggaggcagaggctgcagtgagctgagaccacaccactgcactccagcctaggcaacagagtatgagactccatctcaaaaaaaaaaaaagtacctacctcagagttcaaactagtgaatattaggaagtgcttgagacagtgacaccaaagtgcacaataaatactcgccagtttcattattattaaagaatccatttgaatgtcagctcaacacagcctcctataccgaggcattgtgaaccgcatctccccagcttctccaggcttttccaagaatcagggacactgtagcctgttggtctcagtgtatgacagacacggaggaagcacatctttagctgatacttaaacagagaccctgagcgcacatacacccgcgcacacatgcatggagcttcaccttctctgtcattctgcagtgaccaggagagcaagagctcccacctcccttcaaaacactgtgcccatcccgggcactaaggcctctttaaagcacggcacctccacgagggagggccacagccacatacactccacctggcaggtggacagcgtgagcacgtggaccatagcagggacaaggtgccccggccagccccaacgccctctgccgctgacagggacagaagccctctccagctgcgtgtgctgcagaggccatgcgtagcctccagctgcattctattccactccagtgcctgggccagttagcaccagtgtggaagacagtgagctggctccggacaacagggatggaggaaaggtcccacattcacattcctgatacgtggacaaggtgaggggccgcaatcgctctggcagcattttaaagatggggaagtagcagacacccacgcgtgaaggcaggagagccccaactgtggtggaaatggccccagaatggtagggccaagcctagctccagacaccccagagccctggagaagccaagactgagggagaaagcctgagggaggagcgccccagtccccagggaccggcctggtgcagagctgcagctgatgttcccctctgtgcagccccaccctctgcctcgctgagctccctgctgcgagggcctcgggtgcaagggggaggcaggtctctatctcatggagctgtcagatgagacatcgcgatcggagtcctcagcctcgcttggcggcggcggcgggtcgctaagcgggaccgcagtgaaagcaggagactttctagaaaaaaacaccagttgtcaaccttggggcaggcaggaatcctgaagacggacggcactcctcctcctgctgcctcaccctctggcagcccgtgagaagtaccggaagcgagggcggggccgcgggatggcgagggagcggcagggactgaactctctccaaacccaccctgacagggaaatgggccccgcctgtgtcttgggaactcagaggctgaggtcaggcatgatggctcacgcctgtaattccagcactttgggaggcagaggcgggtggatcacgacgtcaggagttcaaggcaagcctggccaacatggtgaaaccccatctctactaaaaatgcaaaaattagccaggtgtggtggcgggcgccttggaggctgaggcaagataatcacttgaacctgggaggcggaggttgtagtgagccaaaaaaaaaaaaaaaagaaatagctgaagtcacagtaggagagaagctgctgagcctccagcaccctgactctagggccttggctttatgtctatctgcagtatttttgtgatttttaaaaattcactttcttgttgcggtgtaacttacacagggtcaaatgcacaaatcatggccctgactttagataaaaatctgcccccacaacccttctgttccttgccagtttttaaactgcctctaaccaggggaaccaccagagctggtggccttgggaggtttcagccctcccgtcatgaatggacatagctcatccaactgccaagggagagagctgtgggtctgggccagccccaccaggtaactcccaaagggcagccccacagcaagatgtgacccagtcattgcctgagggtctctggggctgtgttccaacctttctccccgctgtgtccccctggaaggccccatgcccaggggaggcgcctaccggtctccagactgggtgatgagccggcggcaggtctccatctgcacgtccaggccgcgcttcatgctgcacatctccatgtactcgtgcaggtgccggttcatgtcgttcttggccgtggccaactccagctgtggtggggcaggcagggccatcggggttaacaggtggcgttcacagcgcctctgttgcccccgccaggaggccaacacgccaagagcagtggctgggccgggggcccaggcagccattactgaaggctcagattttaaaataaaccagaccaaggcagaaggcagcgaatacaccattgtttcctctgcttttcctggaaatctttgcctatggaggaatgcaaaggtactgctgcaggctgtgttcttttaagactgataagagtggtgaggaaagtcagtgtgggcgggatggagcagatccacaggctcccagggccacacaggttcctatggggcccggctcacccctcatcccctgtactgcgctggtcctgggctgtcccctcactgcactgtgagctcccgtaagggttagcgacaggttttcctcatcctggcagacccagacctcgaagggaattaactctctctgtgacccgtggcatacaagcctggctaactgctgaaaggggcccaatcctcttaccattctctccttatcactttattttctcacctaaaagccattttcagacactaaaaggaagactcccatattggaggtgccattgattctgagacctgtcctgatttcagaaatgtcacacgtggaaaacacggggctcagaatcaaagaaatccggtctccctccccgggcccattctgcagcccacactctgaggcaggcggagaggcagggcccaggcagcacaaactcagtgccagggacagtcgactgcctctctttgttttgggtacaaagggcaggggccgcaaggggaagcaaaatgaaacttcctggagatgacgttcgtgactgcctgagtgaagagaacggggaggataccagctgccataatgtggattggacagagagggtgggaacttagagaaagaaggaaacaggttacagggccagacagcagagatggtcaggacagtgacatttggggtgagatggcggctggctgggtgagaagccctagtggggacagcttccctggcaggcttggatccacagctggggccccacagtagtgggaggagcaggacacggtgactgttcaacccagggggaaaatacagtgccaccagcccttgttcagatcagctgcctacaccccaatttctccaccttggaagaaccggagtgcagggagctgggttctggaaccatcactcagggagactgggaagaaggaaatgaaccaaatatattctactgaaatacaaggctagtactgccaccagtagtctctgcatatgtctatgtccccacttaattcacaaatttacagcaggaacaaggtcaggaggtgaacgtgcatgtcaggagatgtccttgacaagccaccttgaaaactgcaggctcgctgtgtgaacagacttgggggtgggacgggaggagacagagagaggtgaggagcggtggggaagaactgcctcccgcccaccactctgggccttggctttaccttgctccatcttagtgaccctgagagatcaaaggtcacaaagataaaggcaccttgggtactcctgaggtcagcaagtcctcctaacggttcccctaatgcagaagtccccaaaccccaggccacggaccagtacctgtctgtggcctgttaggaaccaggccaaatagcaggaggtgagcagtgggcaagtgagtgaagcttcatctgtatttacagctgctccccatcactggcattacagcctgagctccacctcctgtcagatccgcggcggcattagattctcacaggatcctgaaccctaccttacgtgaactgtgcatgcgagggatctaggctgtgtgctccttatgagaatctaatgcctgatgatctgtcactatctcccatcacccccagatgggaccgtctagttgcggaaaacaagttcatggctcccactgattctacattatggtgagatgtataattatttcattatatattataatgtaataataatagaaataaagtgcacaggccgggtgcggtggctcatgcctgtaatcccagcactttgggaggccgaggtggtggatcacctgaggtcgggagttcgagaccagcttgaccaacatggagaaaccccatctctactaaaaatacaaaattagccaggcgtggtggcacatgcccgtaatcccagctacttgggaggctgaggcaggagaattgcttgagcccgggaggcggaggctgcagtgagccaagattgcgccattgcactccagcctgggcaacaagagcaaaactctgtctctaaaaataataataataatttttaaaaatagtaataaagtacacaataaatgtaatgtttgaatcatctcaaaatcatttcccccgggtcagtggaaaaactgtcttccacaaaccagtccctggtaccaaaaaggttggggactgctgcctaaagtcattgcactgggagagaggaggagtcagatgatttgaaaatacttttttttttttttttcctgagatggaatcttgctctgtcacccaggctgtggtgcagtggcacaatctcagctcactgcagcctccacctcccagattccagcaattctcctgcctcagcctcccaagtagctgggattacaggtgcccaccacaatgcctggctactttttgtatttttagtagagacggggtttcacatgttggtggccaggctggtctcacactcctgacctcaagtgatctgcccgcgttggcctcccaaagtgctgggattacaggcgagagccaccacgcccagccaaaaatagcttaaaaggcatgattccatttaagatattattttcgttctttcagatcatgttttacactgggcacttgaggtccagggaagactagggagagatggcagcccgagatctcttctcacgaagatggccgcacaaaggcccctagttggttgaaggcacagtgaaaacacaaaccttaaatccaccatccaaaagagggagacaagaatcaggtcaggcaggaggaggaaacagtgagccgcggtagacgggagggtggggagtactggtttcccttgccatgtctttagctagcagcacaaatccaggaaacctcatctacctggccaagaaaaccaggtttgatgcaggagcaggacagtttgatgtgtgttgggagaatgagtatataggaaaataatttctcgacttccttctcaaacacctccaatgtgacagcaataagaataaagtcaaggccgggcgcggtggctcacgcctgtaatcccagcactttggaaggccgaggcgggtggatcacgaggtcaggagatcgagaccatcctggctaacatggtgaaaccccgtctctactaaaaaatacaaaaaattagccgggcgtagtggcaggcgcctgtagtcccagctactctggaggctgaggcaggagaatggcgtgaacctgggaggtggagcttgcagtgagctgagatcgcgccactgcactccagcctgggcgacacagcgagactacgtctcaaaaaaataaataaataaataaataaatgaaaagaataaagtcactctctccttatccactggtttcactttccacggcttcaggtacccaaggtgaaccttgatctgaaaatattaaatgaaatattccagaaataaacaattcctaagttttaaattgcacgctgttctgagtggcctgataaaatctcactctctctgccgggcgcggtggctcacgcctataatcccagcactttgggaggccgaggtgggcggatcatgaggtcaggagatcgagatcatcctggctaacacggtgaaaccccatctctactaaaaaatacaaaagaaattagccgggcgtgatggcgggcgcctgtagtcccagctactcaggaggctgaggcaggagaatggcgtgaacccaggaggtggagcttgcagtgagccgagttcaagccactgcactccagcctgggtgacacagcgagactccgtctcggaaaaaaaaaaaaaaacctcactctatccagaccaggacatgaaccctccctctgtcccaaggatccacactgtctatactgcctgcccattagttacttaggagccatctcagtgatgagattgactgtcgtggtatggcagtgcccgtgttcaaggaacccttatttttacttcataatggccccaaagcacaagagtagtgatgctggcaatttggatatgtgaaagagaagccaaaatatgctccctttaagtgaaaaggtaaaagttttcaacttaataagaaaaagaatccgatgctgaggttggtaagctctacagtaagaatgaatcttctatccgtgaaattgtaaagaaggaaaaagaaatttgtgccagttttgctgtcacacctcgaactgcaaaaggtaccgccacagcgcacgctaagtgcttagttaagatagaaaaggcattgaatgtgtgggtgggagacatgaacagaaacacgttccagttgacagcaactggacccaagctatcaggaggtcagtgctatctttggtttcaggcatccactgtgggtcttggaacacatgccctgaagataaggggggactacgtatgaatctcccatgtgggcctgaagagaggctgagacgcagagaaggctccatgcctctcccaaagtcagagcgtcgggaaaggaccacctcaccaggccaggagagctcacgggggtcccgtccatccctcctcccacagcaacagcaacctccattctgtggttccacaaatccttcttcctttgtttgtatttttgagacggagtcttgctctgtcgcccaggctggagtgcagtggtgcgatctcggctcactgcaagctccgcctcccgggttcacgccattctcctgcctcagcctcccgagtagctgggactacaggtgcctgccaccacggccagctaattttttttttttgtatttttcgtagagacggggtttcaccgtgttagccaggatggtctcgatctcctgacctcgtgatcttcccgcctcggcctcccaaagtgctgggattacaggcgtgagccaccgcgcccggcctccacaaatccttcttgcagctcctaaagcctagagtttgagctaaagcaccagctccacttgcctctaggtacctctagaaggtacttcaaggcttgggaaaaagcatggagatttctctgttatctcaaaggctgagtggaagaaacaggaaagtcccagtccatctctgagtgccatggcactgatagtgttaataataataaaacagtccccactatgctacactcgctatgagccagacactgccctaagctctatatatgcattatttcatctgatcttctaaacaaccctataggtgaggtattattaaattcctcattttacaaataaaaaggcccggcacagtggctcatgcctgtaatcccaacactttgtggggccgagttaggaggactgcttgaatccagaagtttgagggcagcctggaaaacatagtgacaccctgttctctgtaaaaaaaatcaaaaaattagccgggcgtggtagcacacgcctgtggtcccagctacttgggaggctgaggtaggaggattgcttggatccagaagatcacggctgcagtgggcctggagtgacagagcaagaccctgtctcaaaaaaagaaagaaagaaagaaagaaagaaaaagaaacaggtgtagggctggacacatggctcatgtctctaattctagtactttgggaggccgaggcaggaggatcacttaagcccaagagtttaagacctgcctgggaaacacagggagacaccatctctacaaaaaaatacaaataaaaattagctgggtatggtggcacacacctgtgggcccagctactcagaaggctggggtgggaggatcactccagcctaggcaacagagtgaaacagagtgagaccttgtctcaaaaaagaaaggacagaaagacaagggagagagggagggagggatagagggggaggggaagagagagagagagagaaaggaaggaaggaaggaaggaaagaaaggaaaggaaaggaaaggaaaggaggggctcagagggaacaagtaacttattacaagttcagggtggttgtaagtaacagagctgagatttgaaccgatatctgtctgtttccagagcctgaggtcttcagagcccaccaggaggactatagctgagagatcactcctcctcccaagaccttggtctctagggtcctgcagcagggatgaggccactgcgcctgcagccccactcaaaaccctctgggacaccacgcccctggcttccgctcctgcccttac

SEQ ID NO: 18 - DNA, 16312 bp, Mus sp.
gtgagtggtcctgttaggttccggtgcagtaaacttcatctgcctccctaagtgctctgagactcctgtctgaactttctgccttgctagcctcactacagctatggaaccagagtgactaagaccagatcttaattcctggtcccttttttaaaggccattcatttccttttttgttaacatgcattggtattttgcctgtgtatatgtttgcatgaaggtgccaagtccccttggaattggagttagactgttgtgggccctgagaactgaacacaggtcttttgtaagagcagccagtgctcctaaccactatgccaccttcccagccccagtgaaggccttgagatagggtcttgctatgtagacccagctgcctcagactaacagagattcccctgcctctgtctcctggtagtagaggtatgccgcacatgtttgcactggactgcttctttagagtagtttcatgtttaaagcaaaagtcagtaagtagagctcccatatgctctgtcccacacccgcatggcctccaccccatgaccttcttgtcctggtatacgtggtcctggatgaaggaacgtgattgacagatcagcatccaaagtccactcactggacacagtggcacatacttaccacttgtgtcatactgggcattttcaggactttgaggtcaccttaccccccacccctgtggtggcatcagttagttggaatcacagtttcagattggtttctttagttttctctcttttcatggctatttctttttaagcagggagataatgtttccagccctgtctttaactttctgtcctccaggctataatagatgaatttgagcagaagcttcgtgcctgtcacaccagaggcatggatggaatagaggagtttgaaactggccagggaggcagccagcgagccctgtctgccaagaaaccctccgctggtatgagagggatgcaagcccacatggggctcctatgagtggtccccaccctttttttgttcctttgtggtttgtttttggttttgaaatggtccccttgcctttcttttctagtctcaaggctacagcctctgacttctgtagactcagacaatgactttgtcacacctaagccgcgacgtaccaaacctggtcgtccacaaactcagcagcgcaagaagtcccagaggaaagccaaagttgtcttcttgagtgacgagtccagtgaggacggtatgatacaccctcccaacagatcagctgcctgtgtggggtcttccagtggggcttgcctagctgtggattcattcttcctggccatgtcttcctcactatgcagctcggctgactttcacagtgtgccgtgtgcgtgtgtgtttgtgtgtgtgtttgtgtgtgtgtgtgcacacgtgtgcgcgtgcgcacgcgcgcatgtgtttactttgagatccattttgccccatctttatgactcagcttttgttcctataattctttgaatagaactttcagcagagatgacagaagaagagacacccaagagaaccacccccatccgcagagcatctgggcgaagacacaggtcctaggagctgcctttgcctactcccgtccctgccgtgcagggtatcttactggatgatcttggattcagtgcccccccccccttgtagaagtgcttgttgtctgtggttttaacatggaaatggcctttgcattgaggtctttctcttagagccctgctgccactttccctttctccgagcctgtgccaattgctgttttctaatgaataaatgtttttatagacttttaagcatcctgtcctggctggtctgtcccatccttagccctgagctgtgtctggatcttcatggcccctgtggtgcctcatccttgcacaactgaaactctgctgtgcagaattatgcggtttctaggttcacgtgaacagttctcagcagtctggggtactttgcaccccagaaaacattcggtaatacctagagacttatttctgtttgccttgtggggcagggggctgcagtgaaagccttaagatgaaggccaccttcatgcacattgtgccccataacacagcatgtcaggagtacacaacttgagaaatgctgctctagacctaaggtttgtttcctctgtgcgccccaccccatcccccgaagactggcaaatgactcagcctggctaatggcagagattatgaaccccactcagttctataggtgacaaggtccatcgccccagagccagtccttcactacactaaggggtctgcaggaggtccagctgtaagccatgctgtgtgcccaccatccccaagcccttaatctgctgagtcacttggagcaggaggcacaactgctgtttctgctgccatgacaacagcacagtgctgctgaagtgctccctacctcttggtgaggggacagtaatgatagatgacaggcgtgaggcaaaagggaaggtgcacctctgaaacactaggttagggctgggctatggaagggctctttgggtaggaaaagggttaacgctgtgcatagagcctcggtaggaaggcagaaaagggactctgaatctgccatgcctctcctccccaccactgcgctggggccactcccacccttcccaccctgttcatctggcctgctccccgctcatgggaagcctgggcacgggccacacagctgtcagtctaacacagccttggctctggggccccaaggaggtagggcaatctggaagggcaaggagccaagactagatttggggtgcagcccagcgtgctccctgccttttaagcaaaggttatcaccaggccagctaaacttagcaataggctcttgagctagatgagcagggggctggtttcatgttgcactggcctagcaaagaggcgccagagtccccctgccctgcacctgctacagtgctcccaagcccttccacccttcccagcctccctacccttcggagtgaagaatcccggtctcgaaaagacaaaataaaaccaaagaatgccttttctcccttccacttgtggcaagaggctaggggcttccctgtcctggctcaggggtataatatttcctctcctgtgttctcccctcactgatctcaccctgtgtccacgagggcagccaaaaaaggagagtccctggctccagggccagtcagcacgtccctggatggggagggacttccgccctcacgtcccaactctccaccctggggctacagtgggtgaaaggggcagtgtctcctagcctgggcggggcagctctcaggttccgaggagggatactataggaggcctctgagcctcctccaattcaacccttaagagggatgctgcccttaccccggggtcccagcttaggttcatcaggtaaactcaggagagtgtttgtaagtctcaattatcaggtttccaggctgcagggcatcctgacctatggcgtagcaatctccttttcaagcgtcttcctggccacccccttgccctcacagcgcggtacacaagccaggcctctggccaaagacagaagccaggaggggggggggggcagatagggaaatggggctcctgagggctcccgcgggaggaaaagtgcccccaccctggcattttcttccactcttttttttttcgggggggggggttgctgtgtcactaccgaagaacaacgaggagaagatcctcaacttttccgcagccttttcaataatggggagaggttcgatgatgcagtggcagggagacccacacttctccatttcccctgttctcccattttactcgggaagcagcattcaggtctctgggtcctggatgtccttggtgcacactccaaggactcctcgtccttaagttcatagtctgtattccctgagtcctatcctgggaaccatcacccggtcacctcctgagcggggcaatctcagctcccctccccctatcagttcggagcccacacgcttggtgcgtgcacatttcaaaaatgaggcgggtccaaagagagggaggaggggaaatgagagaggcccagctactcgcggctttacgggtgcacgtagctcaggcctctgcgcccttgagctaggactggataagcagggcgggaggcggggcgcgcgtcatcagctcccccccaccatccgggttcctataaatacggactgcagccctccctggtgctctctgctcctccctgttccagagacggccgcatcttcttgtgcagtgccaggtgaaaatcgcggagtgggccgcaggaggccggggacagtcggaaactgggaaggggagtgggcactgtacgggtctagggatgctggtgcgaagtgtgcaagccggacccaggctccgcattgcaggggggggatgatggaggacgtgatggggcgcacggcgggaatggaggcggggtgggggaggggactgcctggtgtccttcgggccacgctaatctcattttcttctcctgcagcctcgtcccgtagacaaaatggtgaaggtcggtgtgaacgggtgagttccagggcggggccctgctccgttgcctacgcaggtcttgctgacccgggggctctgcagtactgtggggaggtggatgaggtggccgaagcgcccaaggagacctcaaggtcagcgctcggacctggcgatggctcgcacttgcggcccccagctgctgcacctctggtaactccgcctttgcggggatgagcggcccggagtcttaagtattaggaacaaccccacgcgcccgttcagacccatcccgtaatccccagtccggggcttctttctttactttcgcgccctgaggagtcacgtgccaggagggaagcccctcccccatctcccccttcctcggggtcggtggcatggcgttgtgaggtgcatacctttgcgcatcatctcccaggcttgggcttcctttagggtaactggccgccgccatgttgcaaacgggaaggaaatgaatgaaccgccgttatgaaatcttgcttaggccttccttcttcctagcttgtgactaacctcattcctctcggctgggtggagtgtcctttatcctgtaggccaggtgatgcaaggcttccgtgctctcgagagagttctacctcacaatctgtctcaccttattagccttaaaagcccttgagccttattgtcctcgggcataatgcgtattctagattattctctgaaaatcaaagcggacttacagaggtccgcttgacctcccaaccccagaggtagttatggcgtagtgcagagccgtgggatggggagctgagtcatggtggttctgaaaagaaattttccaccacaaaatggctcctgtagtagcagccccttccatcccctgcacttcccatcacagcctcgcactgacccaggccctataggccaggatgtaaaggtcattaagaggattgggtgtccctgcgcctcagaatcctgcccttctccccgttccatcctccagaaaccagatctctccactccgccctgatctgaggttaaatttagccgtgtgacctttctggatctggggtctgagcgggctctccaccctgctccccctacacacatctgttgctccggctctcatttttgcccgagaagaacaggtgtttcgcgaacgagccctgggattagggttggaaaccccccacatgttttctcagtctttccccttagttcgagggacttggaggacacaggtgggcccgccctgtgctgctcacgctgacctttagccttgccctttgagcttgctgatgaatgagttcacaggtctgccctgtccagggggtgtagcctgaagtccagccatgctggaacaaacttcccagggcatgagtgatgggggtgatgtgccaagcttgtacccagactggggcaaactgccacttcttaagagacttagaatgacttggaggaggtttgctgggcaagcaatcacctcttggacaggaaagaaacctccactttataaccgtgctataaaagccctgccaggcctcggctgctcaaagaatataaaattagatctctttggactttctagggtgggaacagtctatatttgggttgtacatccaagcattcaactagctttattaaagggaattctgaacaaaacatgaacttcctgatgcatacatttctaatgtactgtgtctgcataaaggcttgtacctttgttgtggtacgtgcatagctgatggctgcaggttctccacacctatggtgcaacagtattccactctgaagaacatgagatagcctggggctcactacagacccatgaggagttctgatctcagctcccctgtttcttgtctttcagatttggccgtattgggcgcctggtcaccagggctgccatttgcagtggcaaagtggagattgttgccatcaacgaccccttcattgacctcaactacatggtctacatgttccagtatgactccactcacggcaaattcaacggcacagtcaaggccgagaatgggaagcttgtcatcaacgggaagcccatcaccatcttccaggagcgagaccccactaacatcaaatggggtgaggccggtgctgagtatgtcgtggagtctactggtgtcttcaccaccatggagaaggccggggtaagtggccggaaagctgaaggtgacgggcacccttgatatggtgcaacctgaaaaccaagaactgagtctgaaatcaacttctttcccttaaacaggcccacttgaagggtggagccaaaagggtcatcatctccgccccttctgccgatgcccccatgtttgtgatgggtgtgaaccacgagaaatatgacaactcactcaagattgtcagcaatgcatcctgcaccaccaactgcttagcccccctggccaaggtcatccatgacaactttggcattgtggaagggctcatggtatgtaggcagtggggagacagctcatgcatttcttatcttaccctgccatgagtggacccttctttgtaggtgtcccttttgggtagaggggtgccgtgcaggacctcactcattgcccccgtgttttctagaccacagtccatgccatcactgccacccagaagactgtggatggcccctctggaaagctgtggcgtgatggccgtggggctgcccagaacatcatccctgcatccactggtgctgccaaggctgtgggcaaggtcatcccagagctgaacgggaagctcactggcatggccttccgtgttcctacccccaatgtgtccgtcgtggatctgacgtgccgcctggagaaacctgtatgtatggggagagctgggcttgtctctggtgtgacagtgacttgggacaaggatagtcattttggggtttgttcttatcaggccaagtatgatgacatcaagaaggtggtgaagcaggcatctgagggcccactgaagggcatcttgggctacactgaggaccaggttgtctcctgcgacttcaacagcaactcccactcttccaccttcgatgccggggctggcattgctctcaatgacaactttgtcaagctcatttcctggtatgtggggatgggaaacctgacttttaagagcaactgggggtttggtgccctctggtggctagctcagaaaagaaacccaaactaacagttgtcccaatttgttctaggtatgacaatgaatacggctacagcaacagggtggtggacctcatggcctacatggcctccaaggagtaagaaaccctggaccacccaccccagcaaggacactgagcaagagaggccctatcccaactcggcccccaacactgagcatctccctcacaatttccatcccagacccccataataacaggaggggcctagggagccctccctactctcttgaataccatcaataaagttcgctgcacccacctctccttggttttaatgcggtgttgggggagggagggtgtgtatggggaggtgaagcaggctcaatcaacttttgtgcatgtatctttattggctctgacatccagctgggtgccggaagtccagggctacattctatccaagtcccagcccagggcgcctgtcctgaacagctgatatgtctggcaatgcggagggtggggcttgacaatgggacaggaaatgcagtctgagactcacttgttggacagcactgacttccagagctcactgctcttgagaaagatcggtgtccttttatctgcaaacaaaggttaattgtacccagccctcctgcagacagacgtaaaattagataaaattggaagggcgcccagtacagtaaaacctagtggtatccatgaagaggggctgtagcactggtcgatgctttggttcgtgcgtcacacagtgactcagtgcttgggaagtattccacaccgtcattgcttttaaaggttccattccaatagttcaacatagcgtctcctgggaggtcgtgactgactccctaggtcagggacatcttaaccttttggttttgtcagtgacagccatggcggcataccctcagctgctcactggagaaacaggccccgagcaccagtaacctgcaggccacacccatgtgcgggcagcagcttcacctcctcagccgcgacctgggcccagcacaggacttcccaaaggcgagcctgcacggccatgttcactgcaagaggcaagtgaacagctgggagcatgtggacagagcaccccacaccctccaaagccttactagggagcaaagccctatcccaaggtacatgatgaatctgcttcacacgagtgcctaggcaagttagcaccagtacaaaatccaagatacccaaggaatacaggcaggtcccgtgatcacattccttacaaagtggaaaagggaggctcacaagtgtcctggcagcattccgaagatggagaagtagcagatacccacatgtgaggaaggcaggaaggagtgtcccaatgtcatgggcgttgtacagggagggtggggctgagcctagctccagacaccccagaacctggggagggacagcccagggccaggcttcatgcagtggcacagaagacccacacccctgcagacccacacccctgccccagtgagctctggggtgccagacactggtttctatctcatggagctatcagatgagacgtctcgatcggagtcctcagtctcactcggcggtggcggcgggtcgctaagcgggaccgcagtgaaagcaggagactttctagaaaaaaacaccagctgtcaccttggaccaagcaggaaggctgcagacttctaagcttgctttctcatggccttctgaggtacagggggtgccaggcaagggctggggagggaagaagacacttcccttttcaacttgatggggagcagtgcctctcttatcttggggggctctgagaggctcagagatcagaaaagtgcgaagtcacagcacggagatgccccctgagccacgctggggccttagcttcttgtccagcagtaatctggtatgggggtgggggctgctggaccccaaaactcctgggtgcaagcaatcctcttgcctccaaagtggctggggtggagccacagatctcttgtaagcatgcacactatactcagcttgatgtaatttgggagattgggttgggttttttgttgtctttgagacagggcctgtacgcattcctgtctggcctggaactctatacagaggtgaggctggcctggtacttggagtacctgcctgttgcttctatgcatttgaatgacagatatgcgctagcacacctgacttactgtagtttcttggattttgtttgttgttgctatttggtttgtttgggacaaatttttgctgctgtcgtccaggacagcctaaaactcatgacaatccttctgcctctgccttccaagactgaaactgcagacatgtaccactctgcctggtttgcagtaaattttaaaaaaaatttttaatgtgtgtgtgtctgtgtatgtgtatctgtctgtgtgtctgtgtgtgtatgtgtgtgtctgtgtgtgtatctgtctgtgtgtgtttatctgtgtgcctgtatctgtctgtgtgtatgtctgtgtgtatctgtgtgtctgtgtctgtctgtgtgtgtatctgtgtgtgtgtgtttgtatctgtgtgtgtgtctgtgtgtctgactgtctgcctgtctgtctctgtgtgtgtgtgtgtgtgtacacagggccccacacaggctaggcaagtcctctaccactaagctatactccgaaaccccagatttactcttacacaaggtcaaacacataaacccgtgatttggataagatcctgtccccgtatctttggtccttgcctggtttaggttctaagacactcagagttgggacactggtcagtttcatctgatggccaaggcagggagaggaagcttcattctgggccacacctacaggcgactcccaaggacagctggacataaagatgtgacctggtccctgcccacagaccatcggggcctattccagccttcctccctgggagagagacccagcccgctcggggcaggcccttaccggtccccggactgtgtgatgagccggcggcaggtctccatctgcacgtccaggccccgcttcatgctgcacatctccatgtactcatgcaggtgtcggttcatgtcgttcttggctgtggccagctccagctgcagtaaggtggggtcagggaggaccacggttagcaaacccaggcaaggccctgggggccaagcagctcaggagacaagtaagctacaactgcctgtttcccccaggatatggaggaggggtggggcaactggcgatctgacttcaggccctcactgtggctaggcaagggctcaggacagtgctccccgctcggccctgtcctgccaatccactcctactgccacccaagaatcaatcagctgatgtctgagttctctgtaagaacagcaagagcacacaacacagtaaggatggtgcggtgcggtgcggtgcggggagcacgcatccctccagcgcttactcggctcaggcagcagggtcccgaaggcaaacctaaatataaaacccggtctcaaaaaggggtaaggaggccacaccaggcggctcagtggataaggctcctgcgcatctggcaatgtgaatttgttctctaacctccacacatgcaccgtggcatgtggagagagagagacacacacacactaataacaataataataaaatttagtattttgggggctggtgagatggctcagtgggtaagagcacccgactgctcttccaaaggtccagagttcaaatcccagcaaccacatggtggctcacaaccatccgtaacgagatctgacgccctcttctggtgtgtctgaagacagctacagtgtacttacatataataaataaataaatcttaaaaaaaaaaaaaaaaatttagtattttgtttgtttgtttttcgaaacagggtctctccatgtagccctggctgtcctggaactcactctatagaccaggctggcctggaactcagaaatgcgcctgcctctgcctcccgagtgttgggattaaaggtgtgcaccaccactcccggcaaggcagcagttcttaacctgtgagtctcaatccccttgggggttgactatcagacactctgcatgtctgtatttatgttataattcgtaacagtagcaaaattagttatgaagcagcaatgaaatagtttatgcctggggccaccacagcgtgaagcactgtgttaagggtcacagcgtctggaaggctgagcaccactgctttaaggacagacagggccactcatctgtccatctttgccactgagcctcccctcaactgaagctaattcactgtgaaatattccaaagcctcgctgcaatggagggaacctggctgcttctgtttatagcatttcattccacatcatcttctcccttatatcctgctcgcccacataagagagagaccttcctcctgagtgattacagggtaaatccccaacaaaagaaatggggggggggagtctctgtatcaagaaaagtctcccttccagagctgactctgcagccaggtttgaaggcagcaaaaaaagtaggctgaggcggaaaagtatctaggccagagacaaactgcggcctctctttgtttgtgatacaaaggccaggggccagatagggagaatcagactgcggtggggagatttgcttggctgcctgagggcaaagacaaggaggccgagtggccttgaagcatgtggatcagctgtgggaagtcacccgaaacaaatgtgaactggaccgggtgggagtgggccccaggagcagacaaaggagacagccaggacactggcattttgggtcatctgctgaacagctgggtggtaagccacagaggagatggcttgaaaggcatgcttgttcccacagcagactgcaccatttgtgagaaaaacatgacactgaggatcagagctcagctgccaccccccacagacaaatgcacacagcattccaggtcagagatcagctgccacccatgtacatcaacacgcacacacacacacactattacaggtcagagagcagctgtcacccatacataccaacacacacacacactattacaggtcagagagcagctgtcacccatacataccaacacacacacacacacacacacacacacactattacaggtcagagagcagctgtcacccattcataccaacacacacacacacacacacacactattacaggtcgagagcagctgtcacccataaatacaagcatacccctcactccctcccctaccccacctccattatagtctctcaaagcataaggcagtactaccagccatgtctctaagtgtcatatgtcaccctgatttacacactgaaggggaagatgcacattctagaagatgccttcaacaaatcacccccaaatttcagactcagtgtgggagaggcaggggtagaggtagataggagcgatgaaggggcggggccatggaagaaaactccacccaccactctgcatcaccattcaataatgggggctggtctatctctatgccaataaccctgagatcaagggtcatggagataaaggtcctacagatgctccagggagacccaggagctactctgctaccaacgctaccaagtgagcatcctttggctttctcctcaactcattctcccaggagagaacagaagtcatacaacttaggaacagcttcaaacatgttcccaatttttttgggagggcagggggagagtagaggtttgaagcagggcttctctgtgcagccctggccatcctggaagtagttctgttccccaggctgtcctcgaactcacagagatcctcctgcctgttttcccactaccaggactaaaggtggcaccaccacactccattttataggtactggtatcaaacctagggcttcacgcatgaaggtaggcaggcattctaccatgagagcacagtcccagcccagggtgctcttctctgtcctaccacatcatgtttacacttagttatttgtggcctaagggatgaaccacagaagggcagcagctcctgatcactgtgctaagactcctacctggctgacggtagggaaaaaaggaccaagccttaaatctaccgtctgtaagaggtggacacaacccagggagggggacaggaaggaggaaggagcagtaagaggagcggacaggaggagatgggagatcatttgtccccaggcaaagcttaactggtggcagaacactggctgggtgggaattctgggttaaaaaccaaaagcactggttttcctcagagtagcacggccatctgacatgtgctggccactgcagaagagttacttccaaagttccctttcaaaatgcaaacagagtgacccccatgggcacgggaatgcaccacagggctcgcagggagcccaatggcattagtctcctgcctcatccctgcatcagtgtctaagacatagagccctgttcactgtccaatggcgacccccttcctgcagtccctgcctgcagtccagcaggacaatctaaaccccagccccaccaccattcatacccgatgccccaaggctcaaaaaacaaaagagggagacacctccaggagctccaaggctgagccaaggagacaggagtccctctgggtgctacggcacttagggtgttcccagtaataaatgcgcacactaccgcatgtcaacctgccagatcctgtcctaagagccacgcgcgcctcactctatctgatcatcttaataaccctgcctgcctaccagcctgtcgggagagattatgaacatctctattggacaaataaagagacaggcccacccttagcagggtagaaaccatctctaaccctaacaggctgaggcaggaggattgcctcaagtttgactggctacacagaaatctggtctgggtgggggcttaggatggctcagtagttaagagcatgccctgttcttgcagaggacccaagtttattttccagcgcccacatcaggttcacaaacacctgtgagcgccagctcctggaaacctgatgcccacctctgacctccaaggagagagggggagggagggagggaaggaaggagggagggagggagggagggagggagggagggagggagagagagagaaagagagagagagagaaagagagaggagagatgggggcgtggatctgcgaggaaggcaggcaggcaagatgaatgtgtgtgagagcctacaacaaaagaataaaaaggaatgaaggagctagctgaggaacacctcatcagtgggaggcttactgcacagcatgaagacctgagtttgatcttagaacccatctagaaaataagctagggctgaagagatggctcagcagttaagagcactgtgtttgctcttgcaattcccagcaaccacatggtagctcacaacctgggacctgatgccctcttctggcatgcaggtgtacatacagatagaacactcatatattaaataaataaataaataaataaatcttaaaaaaataaaataaactagccatagtagaacacacttgcaatcccaaagctaagaatgtacagacagacagacagacagacagacagacaaaagatgcctagggctccctggccagctcggcctactcagcaagtttcagaccagtgaaagaccctgtctcaattaaaaaaaaaaaacaaaaaacccttaaccacactcaaagaacactacccaggtttgtcttctggcctccacacttgtatgtgtacacatgtgcgcgtgcacgcacacacacacacacacacacacacacacacacacacacacagaggaaaggaggaagacccaaggaaggataaaaagctggtcagtcaaagctggcttaaagctcctgccgtaatcctatcgcacaggaggcgtgggcagaagcaccaggagcttgaggccaacctagactacaagacaagatcttgaaaagaaacagagaggcaggggcaggaaggacactcacaagtgcgagagagcttgcagttgtgatgcacacttttaatcccaggagaggcacagtaacttctccagagtgagttccaggacagccagggctacagagaagccctgtctgtcagggggaaaagaaaaaaaaaagaaagggggggacagaaaagaaaaagatagtctggtgggaaatgattcaccgggtaaaggcatgagccaccaagtccaaagacctaaacttgattgctgtcacccccatggtaaaagagaacttcctcccacacgttgttctctgacctctacacatgtgcatggactgcgcatgctctctgacactccatccatccatccatccatccatccatccatccatccagtgtgtgtgtgtgtgtgtatgtgtgtgtgtgtgtgtgtgtaaaataaataaaaaggaaggagggagggacagcaaaagatttgttccatgtggtgacagagccgggatttgaacccacatctatgtgcctccagatcctgaagtctcaaggacctgctacagctaccacaactgagaggatcactactgacaacagatggaagctcgggccctggggttcagcgaggaggaggccagcaacacacctacagcgtggctcctgagccctggacctacagcgtggctcctgaggccggcacccttac

SEQ ID NO: 19 - DNA, 11058 bp, Rattus sp.
gtgagtggacccggatcactggccctgttgggttccggtgtaggaaacggaatccgctctccctaagtgctcagagatccctgtctggacttgctagcctcactacagccgtgcaaccgaagtgacttaaggccagacctttttttttttttttttctgagctggggaccgaacccagggccttgcgcttgctaggcaagtgctctaccactgagctaaatccccaaccccaggccagaccttttttaaaggccattcgtttcctcttttatttattttttagtgtgcactggtattttgcctatgtttatgtctgtgaaggagcccagtgccctggagttggagccacagacagttgtgggtgctgagaactaaacacaggtcttaaccactatgccgccccccccccccccagcctcagtaaaggccattttctacttgcgatagggtcttgctatgtagacccagctggcctcaaactaacagagattcccctgcctctgtctccagtgtgctggtagtaaaggtgtacccccatgttcggcactagactgcttctttagagtggtctcatgtttaaagtaacactgagttcccatctgcctgtcccacaccctcatggcctctaccccatcaccttatcctcggtacgtgatcctacaatctacattgactgggatgaaggaatgagattgacagatcagcatccaaagtccactcactgaatccagtggcacattcttgccactagtgtcatactgggcattttcaggactgtgaggtcacctcactccccaccctgtggtggtatcagtcagttggaatcacagtttcagagtttgtttctttaacttgctgtatctttcatggctgctttttaaatagggagataatgcttccgaccccgtctttacctttttctccaggctgtaatagatgaatttgagcagaagcttcgtgcctgtcacaccagaggcatggatggaatagaggagcttgaaactggccaaggaggcagccagcgggccctgtctgccaagaaaccctccgccggtgtgagaggcaagcccacgctgggccccacaggcagtcccctcttttcttttttttcctttgtggtttgtttttggttttgaaatcatgtccctctgcatattccagtctcaaggcaacagcctctgacttctgtagactcagacaatgactttgtcacacctaagcctcgacgtaccaaaccccgtcgtccacaaactcagcaacgcaagtcccagaggaaagccaaagttgtcttctcaagtgacgagtccagtgaagatggtaagatacaccctcccaacaggacaactgcttgtgtggggtctccagccgggcttgcctggctgtggattcattctccctggcagtgtcttccccactgtgcagctgctgggctgactttcactctgtgtgtgtgtttgtggtgtgtgtccagatccgttttgccccatctttatgactcagcgtttgttcctataattctttgaatagaactttcagcggagatgaccgaagaagagacacccaagaaaaccacccccatccgcagagcatctgcgcaaagacacaggtcctaggagctgcctttgcctattcccatccctgctgtgcagggtatcttactgaatgatcgagaattcaacgccccccccccccttggaaaatacttgttgcctccttgtttttaacatgtaaatggtcttttcgagcaaggcctttctcttagagcccctgactgctactttccaagatccaatcaccatgttttctagtgaataaatgtttttatatacttttaagcattctggccggctggtctgttccattattaggcagcagatagcagtccagagagagaaccccaggcatcctgagccatctctggaccttcatggaccctgtggtgcttcatctttgcacaactgctgagatttgctgtagagagttctgtggtttctaggttcacataagcacttctcagcctgggggtactttgcaccccagaacacgtttggcaatatctagagaccaggggtgggagagatggctcagcgattaggagcacttactgcccttccagaggtcctgagttcaattcccagcaaccacatggtggctcacagccatctgtaatgagatctggtgccctcttccggtgtgtctaaagacagctatataataaataaataaatctttaaaaaaaaaatctagagacctatttcttctgcccaccttgtggggcaggggctgctgtaggcacccagtcagtcagagcctaaggaggaagactccttcatccatagcactcccaacacagcatgtcgggactgtgcaactagaggtgctgctctagacctgcggtttgtttcctgtgtgccacaccccatcccagaagactggcaaatgagtcagcctggctggtggcagagatcatgccattagctctgttggtgacaaggtccacctcccaagagtcagtcctccactatgctaaggggtctctcgctcagctgcaggcccaggctgtgctatgtgctgcccaccttccccaagcccttaatctgctgagtcacttggagcaggaggcaggactgttgtttccgctgtcatgacaacagcacagtgcagctgagcctctccctacctcttggtatggaaagagtaatgacaggcgtgaggcagaagaagatgcacctcagaaacactaggttaggggtggactgtggaagggctcttgagacctctgggtaggaaaagggttaactcggtggatagagcctcagtaggaagacagaaaagggacagctgagtctgagatgcctgtctccccagcactgcactggggccactcctaccctttccaccctgtttttggcctgctccctgctcatgaaaagcctaggcatggaccacacagctgtcagtctaaacacagctttggctctggggccccaaggaggtagggcaatcccagaagggcaaggagccaggactagatttggggtgcagcccagcgtgctccctgccttttaagcaaaggttatcaccaggccagctaaacttagcaataggctcttcagctagaagtgcagggggctggtctcaagttgcactgggctagcaaagaagccccagagtccccctggcccgcacctgctgatggcccgcacctcccaatcaaccccttccaccctttctagcctccctaccctgcaaagcaaacaagtccttttctccctccactgtgcaaagaggctaggggttgcctgttctggctcgggatacttcccttctgctttcttccctttctgatctcaccctgcttccacgagggcagccaaggaaagagagtccttggctcctgggccagtcagcaggtcgcctggatggggagggacttccgccctcacgtcccagctctccaccctggggctgcggtgggtgaaagggacagtgtctccaatcctgggcggtacaactcaggttccggggagggaaggcctctgagcctcctccaacccaaccctcaacagggatgcttaccccggggccccggtataggttcagcaggtaaactcagggaagtgttgtctcaatcaggtttccagactgcaggggatccccgacctatggcgtaggaatctccttttcaagcctctagtcacttccttgctttcacatcgcggtacacaagccaggactctggcctaagagatagaagccaggcggtggggagagaggaaaatggggctgcgaagggcccccaccggaggaaaagtgccccccccgtccctcccccagccttttcttctgctccttttttcggggcggggggggcgtgctgtgtcactaccgaagaacaacgagaagatcctcaacttctcctaagccttttcactaatagggagaagttcgatggggcagccttgggcagacccacacttctgctccatttccctggttcctgcagctctcagattctcccattttattcgggaagcagctttctggtttctgggtcctggatgtccttggtgcacactccaaggactcctcgtccttaatccatagtctgtattccctgagtcctatcctgggaaccctcatccggtcacttcctcggcgggacaatctcagctcccctccccctctcaggtcggagcccacacgcttggtgcgtgcacatttcaaaaacgaggcgggtccaaaaagagggagggggggaatgagagaggcccagctactcgcggctttacgggtgcacgtagctcaggcctctgcgcccttgagctgggactggatgagccgagcgggaggcggggcgcgcgtcatcagctccccccaccatccagttcctataaatacggactgcagccctccctggtgctctctgctcctccctgttctagagacagccgcatcttcttgtgcagtgccaggtgaaaaagaaaagaaaaaaaaggactgggccgcaggaggccggagacgaatggaaattaggaatggggggaaggacgctgtacgggtttaggggcgctggtgcgaggtccggaagccgagcccaggctccgcattgcagaggatggtagaggacgtgatggggcatgcggcgggaatggaggcgggtggggggaggggactggccacgctaatctgactttcttctcccgcagcctcgtctcatagacaagatggtgaaggtcggtgtgaacgggtgagttccagggcggggccccgctccgttgcgcatgccggtctggctaaattcggggctgtgctgtagcgtgcggggaggtggatagggtggccgaagtacccaaggagacctcaaggtcagcgctcggacctggggaaggctcgcacttaacgggaccccagctgttgctccccttgtaactccgcctttgctggggagcggcccggagtctaaagtagcaggagcacccccgacactggcgcgcacccgctcaggatccgacccgtaatccccaggctggggcttctttctttactttcgcgcccagaggagtcacgtgccagaaggggagcccctcccccatcgtccccttcctcagggtcggtggcacgggggtgtgaggtgcatacctttgcgcatcttctaagggcttcacttatggtaactggccgccgccatgttgcaaacgggaaggaaatgaatgaaccaccgttaagaaatcttccttcggccttcctcattcttagcttgtgactaactttctcattcctctcagctgggtggagtgtcctttattctgtaggccaggtgatggatggcagggtttccgtgctctcattagagctctactatacaatccgtctctcgtcagcctttataggcctccagccttatttccctcgggcataacttatattctagattgttctctggaagtcgaagtgcatttacagaggtctacttggcctcccagccccggaggtgcggtagcaatggcgtagtgccgagcccgggggtggggtggggagctgagtcatgatggtcctgaaaagaaattttctgccacaaaatggctccggtggtagcagccccctccctccagggcctccacttccatcccagctcagcactgacccaaaccctataggccaggatgtaaaggtcactaagaggattgggtgtctctgagcctcaggatcctacccttttccccaacccatcctccagaaaccagatctccccattccgccctgatctggggttaaatttagctgtctgacctttctgtatctggggtctgagctgggctctctgccctgttcatccctccacacatctgttgctcctgctccgatttttttttttttttttttttggccgggaaagacaggtgttttgcaaatgagtcctgggattagggctggaaaatcactggttttcttgatctttcccccagaaagggacttggaggaagcaggtgggctggccctgtcctgctcactctgacctttagccttgccctttgagctgctgatgaatgagttctctcctgtccgggagtgtagcctgaagtccagccatgctggaaccggcttcccaggcctgtgtggtgggggtgatgaataccatgtgccaagcttgtacccagaccggggcaaaccgccacttcttaagagacttaaaatgactttggggtgccgggcaaggctgtgggcctaatcacctcttggacaggaaaggaacctccactttatagctgctataaaagtcctgccagccctgggtggctcaaggaatataaaattagatctctttggactttctagggagggaacattctatatttgggttgtacatccaagcattcaaccggctttattaaaggaaattctggacaaaacatgaacttcctgctgcttacgtttctgatgctgtactgaatcagcgtaagaacttgcacctttttgtgatgcgtgtgtagcgggctgctgtaggatctccacgcccatggtgcagcgatgctttactttctgaagaacatgcgttggcccagggctgactacaaacccaggaggggttcactgatcccaactaactcgcctatttcttgcctcagatttggccgtatcggacgcctggttaccagggctgccttctcttgtgacaaagtggacattgttgccatcaacgaccccttcattgacctcaactacatggtctacatgttccagtatgactctacccacggcaagttcaacggcacagtcaaggctgagaatgggaagctggtcatcaacgggaaacccatcaccatcttccaggagtacgtatggaaacatgcacagggtacttcgaggagatggtaaggggcggcattaactctgtcctgttccccgcctgtaggcgagatcccgctaacatcaaatggggtgatgctggtgctgagtatgtcgtggagtctactggcgtcttcaccaccatggagaaggctggggtaagtggccaggaagctggaggtaaggggaacccttgatatgggtgcaacctccaaactgaagagctgagtctgaaatcaacttctcctgcacaggctcacctgaagggtggggccaaaagggtcatcatctccgccccttccgctgatgcccccatgtttgtgatgggtgtgaaccacgagaaatatgacaactccctcaagattgtcagcaatgcatcctgcaccaccaactgcttagcccccctggccaaggtcatccatgacaactttggcatcgtggaagggctcatggtatgtaggcaatggagacagctcatgcatgagtggacctttctttgaagatgtccctttgggtaggagggctgccctgcaagacctcacccattgcctctatgctttctagaccacagtccatgccatcactgccactcagaagactgtggatggcccctctggaaagctgtggcgtgatggccgtggggcagcccagaacatcatccctgcatccactggtgctgccaaggctgtgggcaaggtcatcccagagctgaacgggaagctcactggcatggccttccgtgttcctacccccaatgtatccgttgtggatctgacatgccgcctggagaaacctgtacgtagggggagagctgggtttgttttgtctatggtatggtggtgacttggggcaagagcagtcatctttggttttgcccttacaaggccaagtatgatgacatcaagaaggtggtgaagcaggcggccgagggcccactaaagggcatcctgggctacactgaggaccaggttgtctcctgtgacttcaacagcaactcccattcttccacctttgatgctggggctggcattgctctcaatgacaactttgtgaagctcatttcctggtatgtggggaccgcaagcctggctcttgagagtaactgaaggtttggtgccctctggtggccagcttagaaaagaagcccaaactaaccgttgtcccaatctgttctaggtatgacaatgaatatggctacagcaacagggtggtggacctcatggcctacatggcctccaaggagtaagaaaccctggaccacccagcccagcaaggatactgagagcaagagagaggccctcagttgctgaggagtccccatcccaactcagcccccaacactgagcatctccctcacaattccatcccagaccccataacaacaggaggggcctggggagccctcccttctctcgaataccatcaataaagttcgctgcaccctcttcttgtgtgtgaatgcagtgttgtgggggttatgggcaggtgaaggaggctccattcggttttgtgcacatatctttattggctctgccatccagctgggtgctagaggtccagggctacattctaaccgagtgccagcccaggccgcctgtccagacacctgatgtgtctggcagtgtggagggtggggcttgacactggaacagaaggtgcagtctttgaacagcactgactttcagagcttgctgctcttgagaaagatctgtgtcctttatctgcaaacaaaggttaattgtacccagctctcaaagtctccagatgttaaattaggtaaagttggaagggtgcccagtacagtaggactaattgtgtccatgaagagggaaggtccctgctgagctgttcacctgtcttcaggtgtgggcacttgaaggtgctttggttctggggcagtgactagccagcgctcactgttggttgggaatgaaatgaaggtgcattacacggtgactcgactcaacgccttcattgctttcaaaggtcccattcaaatgtcagctctgcatagcctctcctgggagatcctagctccctaggaccagggacattgtaaccttttggttttgtcagtgacagccatggaggcattccctcagctggtcactgaggagacaccctgagcacccctaaccttgtgcaggccacacccaagtgtggcagcagcttcatctcagtggcgacctggcccaggcactggacttcccaaaggcacagccatgttcactgcagtgggcaggtgggcagctagctgtgagcatgtgcagtgtccacgccctcaaggccttcctagggagcaaggtcctatccaaaggtacatgcggaatctgtttcacatgagtgcctaggccagttagcaccagtacaaaacccaaggaaaacaggcaggtcccataaccacattccttacaaatggaaaagggaggctccaagtgtgctggcagcattccaaagatggagaagtagcagatacccacatgtgaggaaggcaggagtgccccaatgtgttgggagttgtacagggggtggggccgagcctagctccagacaccccagaaccctggagaggacaagccagggccaggcttcatgcggtggcacagaagatggtcctctgcagacccacacccctgccccagagagctctggggcaccaggccctggttcctatctcatggagctgtcagatgagacatctcgatcggagtcctcagtctcgcttggcggcggcggcgggtcgctaagcgggaccgcagtgaaagcaggagactttctagaaagaaataccagttgtcaccttggggcaaagcaggaaagctcctgagacttccaaacttgctttctctggccttatcttttgaggtacagggggatgacaggcaaggacgggggagggaaggaaaacttgttttcaacttgatgggaggaacagtttcgctctttgggactcagagatcagaaaagtctgaagtcacagcatggagatgttccctgagccacactaaggccctagcttcttatccagcaataattcggtgtgcggggggctgtggggactgtaggaggtgagactgggggagtatgggggtttagggggctgtttcactctgttgtcactgctgaccccaaattcctgggtgcaagcaatcctcttgccttcagttgattttctttttttttttaatttattttatttatatgagtacactgtaactgttttcagacataccagaagagggcatcagatcccattacagatggttgtgagccaccatgtggttgctgggaattgaactcaggacctctggaagagcagtcagtgctcttaaccgctgagccatctctccagcccctcttgccttcaaagtggctggggtggccacaaacccagttagcgtgctcaccatacaatgcaatttggggggtcgggttgggttgggttttttgttctctttgagatgagtcagtttgcattcctgcctggccttgaactcctcacagaggcgaggctggtctagtacttgtggcaattcacctgcccgttgcttctgtgcatctgaatgataggtatgcgctcataagcctgacttaactgccattccttggatttttgtttgttgttgttgctgtttggtttgtgtgggacaaggttttactgctgtccagaacagcctaaaactcatgacaatccttctgcctccgccttccaagactgagactgcagacatgtaccgctaggcctggtttagagatttctaaaaaaatcttagtgtgtgtgtgtgtgtctgtgtgtgtgtgtgtgtgtgtctgtgtgtgtgtgtgtgtgtgtctgtgtgtgtctgtgtgtgtctgtgtgtgtgctagacaggctagacaaggactctaccactgagctatattccaaaaccccagatttactcttaaacaaggtcaaacagctaaactgtgatttggatttggataatcctgtccctctatttctttgctccttgccaggtttgggctctcagaccctcagagttgggcactagtcggtttcatctgacggccaaggcagggagaggaagcttcattctaggccacacctacagggactcccaaggacagctggacataaagatgtgacctggtcactcccacaggccactggggcctgtccagccttcttccctgggagacagccacgccactcagggcggcccttac

SEQ ID NO: 20 - DNA, 3158 bp, Homo sapiens
agcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagcca

SEQ ID NO: 21 - DNA, 3218 bp, Homo sapiens
aagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagttttgaggaagatgaactgagatgtgtcagggtgacttatttccatcatcgtccttaggggaacttgggtaggggcaaggcgtgtagctgggacctaggtccagacccctggctctgccactgaacggctcagttgctttgggcagttactcccgggcctcactttgcacgtgtgcttacctagtggagacaaaagtacatacctcggtagagcgcgcacgcctgtaaccccagcactttgggaggccaaggtgggtgtatcacctgaggtcaggagtttgagaccagcctggccaacatggtgaaactccgtctctactaaaattacaaaaatcagccaggcttcatggcacatgcctatagtcccagctacaggcatgctgaagcaggagaatcgcttgcaccccggaggcagaggctgcagtgagctgagaccacaccactgcactccagcctaggcaacagagtatgagactccatctcaaaaaaaaaaaaagtacctacctcagagttcaaactagtgaatattaggaagtgcttgagacagtgacaccaaagtgcacaataaatactcgccagtttcattattattaaagaatccatttgaatgtcagctcaacacagcctcctataccgaggcattgtgaaccgcatctccccagcttctccaggcttttccaagaatcagggacactgtagcctgttggtctcagtgtatgacagacacggaggaagcacatctttagctgatacttaaacagagaccctgagcgcacatacacccgcgcacacatgcatggagcttcaccttctctgtcattctgcagtgaccaggagagcaagagctcccacctcccttcaaaacactgtgcccatcccgggcactaaggcctctttaaagcacggcacctccacgagggagggccacagccacatacactccacctggcaggtggacagcgtgagcacgtggaccatagcagggacaaggtgccccggccagccccaacgccctctgccgctgacagggacagaagccctctccagctgcgtgtgctgcagaggccatgcgtagcctccagctgcattctattccactccagtgcctgggccagttagcaccagtgtggaagacagtgagctggctccggacaacagggatggaggaaaggtcccacattcacattcctgatacgtggacaaggtgaggggccgcaatcgctctggcagcattttaaagatggggaagtagcagacacccacgcgtgaaggcaggagagccccaactgtggtggaaatggccccagaatggtagggccaagcctagctccagacaccccagagccctggagaagccaagactgagggagaaagcctgagggaggagcgccccagtccccagggaccggcctggtgcagagctgcagctgatgttcccctctgtgcagccccaccctctgcctcgctgagctccctgctgcgagggcctcgggtgcaagggggaggcaggtctctatctcatggagctgtcagatgagacatcgcgatcggagtcctcagcctcgcttggcggcggcggcgggtcgctaagcgggaccgcagtgaaagcaggagactttctagaaaaaaacaccagttgtcaaccttggggcaggcaggaatcctgaagacggacggcactcctcctcctgctgcctcaccctctggcagcccgtgagaagtaccggaagcgagggcggggccgcgggatggcgagggagcggcagggactgaactctctccaaacccaccctgacagggaaatgggccccgcctgtgtcttgggaactcagaggctgaggtcaggcatgatggctcacgcctgtaattccagcactttgggaggcagaggcgggtggatcacgacgtcaggagttcaaggcaagcctggccaacatggtgaaaccccatctctactaaaaatgcaaaaattagccaggtgtggtggcgggcgccttggaggctgaggcaagataatcacttgaacctgggaggcggaggttgtagtgagccaaaaaaaaaaaaaaaagaaatagctgaagtcacagtaggagagaagctgctgagcctccagcaccctgactctagggccttggctttatgtctatctgcagtatttttgtgatttttaaaaattcactttcttgttgcggtgtaacttacacagggtcaaatgcacaaatcatggccctgactttagataaaaatctgcccccacaacccttctgttccttgccagtttttaaactgcctctaaccaggggaaccaccagagctggtggccttgggaggtttcagccctcccgtcatgaatggacatagctcatccaactgccaagggagagagctgtgggtctgggccagccccaccaggtaactcccaaagggcagccccacagcaagatgtgacccagtcattgcctgagggtctctggggctgtgttccaacctttctccccgctgtgtccccctggaaggccccatgcccaggggaggcgcctaccggtctccagactgggtgatgagccggcggcaggtctccatctgcacgtccaggccgcgcttcatgctgcacatctccatgtactcgtgcaggtgccggttcatgtcgttcttggccgtggccaactccagctgtggtggggcaggcagggccatcggggttaacaggtggcgttcacagcgcctctgttgcccccgccaggaggccaacacgccaagagcagtggctgggccgggggcccaggcagccattactgaaggctcaga

SEQ ID NO: 22 - DNA, 508 bp, Homo sapiens
agcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgat

SEQ ID NO: 23 - DNA, 2650 bp, Homo sapiens
atctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagcca

SEQ ID NO: 24 - DNA, 1963 bp, Homo sapiens
agcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttcctaagcttgtctttgtttcatctttcacattagcccagtttcatgcagcagagagagggttatcagtgcagagagagatgagtgagcccagagtcctagggcctgtcccgggatggcagatgagcttcctgccccgtcactgccacctttcccctctcaacctctggaccctgcacagtgaccagacagcctctctggggagaattatgcagtgcctaggctccagatcagtgcttctgaaccgggggcaattttgtctgccagaggacatctgacaacacctggggcctgttttgttgtcatagcctataggggaagaatgctaccagcatttgtgggaagaggccagggatgtggctcaacatcctgcagtgcacaggatggcccctcaacaaagaatcacacggcccacaatgtcaatagcgtcacagttgagaaaacctgctctagaccaagggttgctttctgccgtgtgcctcaccccacccccactcgtgttccctaatcccatctccaaaggttggcagcagaccggcccaggctcgtggaagttcagatcatgatcccctccagctctgcaggagacaagacctgtctcccagcattcctcattgttcccgggtctgcagagggcgtgagctatgctgcaggcgggctgccccctgaagcctgcgcacccctctccagctcctcaagtcttctctgctgagtcaccttcgaaccggaggctgtgagctggctgtcgtgaccacac

SEQ ID NO: 25 - DNA, 1195 bp, Homo sapiens
tggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagcca

SEQ ID NO: 26 - DNA, 256 bp, Homo sapiens
cctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagcca

SEQ ID NO: 27 - DNA, 1941 bp, Homo sapiens
agcttgagattgtccaagcaggtagccagagagcgccatcagccaagaaaccatccactggtacgtaaggcagcctgtgcgggcgagaccagactgggccctcccctcctgcagtgatttgtttcttcttcttttttaaatcacgttttcctgccttttctaggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaaggtatgatgctcccgcctgttcccggccgagaaggcacacagctagggtgcagagggctggtttccataggacctgctgcgggggcctgagtgtagatgctctgccccactgccgcagaagggcctctcctgtacagcttggattttatttcttctgtgcggtgtgggattgtctcacttgttctctgatatctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagcca

SEQ ID NO: 28 - DNA, 1433 bp, Homo sapiens
atctattttttcaccatctttgtgactcagctttttcttattcctttaattctttgcatagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaaccggaggctgtgagctggctgtcgtgaccacactggtgcctctgctgtcatgacaacagcacactacgtcagtagtgctccctgggcactgagctccctctttgcggggagaagacagtaatgaaaaatgacaagcatgaggcagaggggaagatcacgcttgggtggtgcaggagcatggaggtgctcttaatgctctcaatgagaaagggttaacggtcctggttgcaggaatagctgagtcagaggtggggcttcctccactcccccaccccacccctttcaccattagggaccttcttgccttgctcttgctactctgctctgggtggtcattgtgaaaagcccgcaccaaccatgccagtggcagccagacgaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagcca

SEQ ID NO: 29 - DNA, 17130 bp, Cricetulus griseus
  misc_feature (4674)..(4912): n is a, c, g, or t
  misc_feature (11347)..(13938): n is a, c, g, or t
  misc_feature (14757)..(14870): n is a, c, g, or t
gtgagtggtgcagggtgactggccctattgggttcctgttcagtgaacacagtctgttcttcgagtgctgtgggactcttaacttactgccgtgctggcctcactacagccatttaactagagtgacttgagatcactttctatgtccacttcccggtcctaaaaaaaaaatttattttgtatacattagtattttgcctgtgtgtatgtccatgtgaaggtgccagatctggaacttgagttacaggcagttgtgagctgccacatgggtgctgggaactgaacccaggtcttctgcaggagcagcaagtgccatctctctagcccccagtaaaagccgatttctagttgagacatggtctttctgtgtagctctagctggcttagaatttgctacctagaccaggctggccccaaattcagattcccctgcctctgcctctcatgtgctggtaggaaaggcgtgagtcacatgcttggcactagactagctttgtgtttagagcaaaactaagtaagtagagttcccgtttgtccctgtcctctgccccatcatcttcatacccttgggtactgtgatcttacaatctacagtgacctggatgaatgagtgtgagtgacaatgaatgacagagcagtacccagagtccacagttcacactgatactgggttcactgtacagtggcacatacttgtcaccacagtgtcatactggacattttcatgaccctaaggccacatcacccctgtggtgtcactcagttgtaatttcaccctatatagtgatttcaggttggctttttacttagaatataacatttaaattatgtttcttttttggttttgagttttgtttttcgagacagggtttctctgtattgctttggaggttgtgctggaacttgctctggagaccaggctggcttcgaactcacagagatcctcccacctctgcctcccgagtgctgggattaaaggcgtgcgccaccaatgccccagcatctttctgtttctctgtttttaaagagggagataatgtttccagtgctgtttttaacttttttcctgtaggctaccatagatgaatttgagcagaagcttcgggcatgtcataccagaggcatggatggaatagaggagcttgaaactggccaagaaggcagccagcaagccctgtctgccaagaaaccctctgctggtatgagagacaagcccacacggactccccacaaatcatccctgtcatttattggtttttgtttttaaaatcacatctccctgccatttctagtctcaaggcgacagcctctgacttctatagattcagacaatgactttgtcacacctaagcctcgacgcaccaaccgtcatcgtccaaacactcagcaacgaaagtccaagaagaaagccaaagttgtcttttcaagtgatgagtccagtgaggatggtatgactcgccctcccggcagctgcttgcttgccttggggtctccttggggcttgcctggttgtagagtcattcttcctggtaatgtgttccccactgtgcacgttggctcacctttgcagctctatgttttaagatccactttacaccatctttgtgactcagcttttgttcctataattctttgaacagaactttcagcggaaatgacggaagaagagactcccaagagaaccacccccatccgcagatcatctgcccgaagacacaggtcttaggagttgcctcacctattcccatccctgctgtgcagggtatcttaattgatttttaattcatttcctcccttgtaaaaatgttgcctccttgtttttaatatgtaaatggtctttgcattcaaggcctttctcctagagcccatctgactgctgctttcactttccaacaaacacttaaccaatcaccatgttttctaagaataaatgtttttatatacttttattctttgctggtctgtttcatcttccaccttagcccagattcaggcagcaatagagggttatcaaaccagagagaagatacaccagcccaggcatcctgaggctgtctctgggtcttcatgtttcctcattgattcatcttgcacaactgatgagactccactgtagagaattctcacttctcagcctgggggtactttctacccagagacctatttctgtcatagccttttgggacacataaagcttccttcctgcatagaacaccccacaacagtgtatcaggagtgtacaagttgacaaacaatgctctagacctacggttttcttcctctgtgtgcctcatcccagaagagatcatgactcccaggagtcagcctttactatggggtctgcaggggcgtccagcccctcagcggcaagccatgcccaccctccccaagtccttaatctgctgagtcacttggaacaggagacactgattctgctgtcatgacaacagcacattgccatagaaatgctccctacctcttacgtgtgtggtgggggaacagtaatgacaaaccataggcaggaggcaaaagggaagacggcacctcagaaacatgtgttaggttagggcagaactatggaggggctcctgagactctttgatgggaaagggttaatgctgctcctgaaacctctgttggaaggcagaaaagggacagggctgagtccccgcactgggaccatttccatcctctgcatcctgcccccggctcatggaaagcctgggcatgggccacacagctgtcagtcttggctctggggccccaaggaggtagggcaatcccagaatggcaaggagccaggactggatttggggtgcagcccagcctgctccctgccttttaagcaaaggttatcaccaggccagctaaacttagcaattaggctcttcagctaaaagagcagggggctggtctcaagttgcactgacctagcaaagaggccccaggatccccctgcccagcacctgtggctgagctcccaagcccttcccgagagctcaggatccaccctttccaccctccctactcttcagaggaggaaccccctttctccttcccacttgttggagggggctggggccaggctgttctggcttggggtataataccccctaccccttctactttcccctcctctcagacctcaccctgcctccacgagggcagccaagaaaggagagtccctggctgcagggccagtaggcacgtcccaggacggggagggacttccgccctcacgtccagctctccgccctggggctgcagtgggtgaaaggggcagtgtctcctagcctgggcggtgcaaccctcaggttccgaggaggaacgctctgggaggcttctttgcctcctccaacccaacccacaaccaggacattgtcctcaccccggggccccaacctagaccttaactgaggaacacagaggccagtttgtaagtctcaattatgcagggcatcccgacctgtggcgtagggagcgcccctccaggccgcttccctagcctcctcctggccctcacagcccaggcctctggcccaagaaatggaagtgggggtgggggatggaactgcgaatgcgaagggcccccgcaggaggcaaagtgacccctcccgggccttttctgctccgagacttgtttttgcctgtgtcactaccgaagaaccacgagaagatcctcaacttttccacagcctttgcataaaggggagagggtcggcggtgcagctgtggcacacacgcacttctgctcaacccgcccccccccgcccccgttcctgttccttcccaggttctccccattttatcggggcggcaacttttaggtccctgggtcctggaagtccttagtacacactcttcgtccttaagtccatagtctgtattccctcggtcctatcctgtcccccatcaccgggtcacctccccagcgaagcaatctcagttcccctccccctctcagccccgagcccacacgtttggtgcgtgcacatttcaaaaacgaggcgggtccaaagagagggggtggggaggtgccgagtggcccagctactcgcggctttacgggtgcacgtagctcaggcctcagcgcccttgagctgtgactggatggatgagcggggcgggaggcggggcgagcgtcctcggcgctccccaccaccccagttcctataaatacggactgcagccctccccggtgctctctgctcctccctgttctagagacagccgcatctttccgtgcagtgccaggtgaaaaccgcagagtgggccgcaggtggccggggacggtcggaaacggggaaggggggcgctcagcccgggactgcgggcgctggggcgagctccactgcccgagcccgggctccgcattgcagaggctggagggggaagggatggggggcgcggcggggggcacgctgacctctctttcttctctctccctccgcagcctcgctccggagacgcaatggtgaaggtcggcgtgaacgggtgagttcgtggctgggctagggtggggctccgggtcccgctccgtcgcgtatgcaggtctaccccaccccggggctctgcgggagcgtggggtggccggtgggtggccgcagcacccaaggagacctcaaggtcagcgagccacctcctcccttgcggggatgagcagcgcggagtccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaggtcggtggcaccgggccgtgcggtgcccgctttagcgcatccatcatctcccaagggcttcctttagggtggctggccgccgccatgttgcaaacgggaaggaaatgaatgaaccaccgttaggaaacctcccttcggccttcctccttcctagcccgtgactaacctccccactccctccccgggtggagtcgcctctgtactgtaagccaggtgatgcaaggcttccgtgctctcgagagagctctacctcgccagctgtctcatattattagcctcaaagcagccctcaagcctcatttaccttgagcatatgatatattttgtagattctctgagaatcgaagcggacttggagaggtctgcttgtccttctcccagcccaaaggtggtagctatggcgtagcgccggaggggggagtggggggggagctgagtcatggtggttctgaaaagaaaatttccaccacaaaatggctccggtgctagcatccccttccccccataacctctgcttcccatcacaccctgacccaaaccctgtaggccagactgtaaaggtcactaagaggattgagtgtctgagcctcggaaccctgcccttctccccatcccatcctctggaaaccagatctcccccgctccaccctaatctgaggttatatttagccggctgacctttcagtatttggggtctgggcccctacacacatctgttgctcctgctcctgatttttagctagcaaattcaagtgctttgcaaattagagcccagggattaggggttggaaagctcagtggttttctcagtctttccctttagggggagggacttggaggaagcaggtgggccgacccctgtcctactcattctgacctttaaccttgccctttgagcttgatgatgctgagtgcacgagttcttcctgtccagggggtgtagcctgaagccaggccaggctagaacaaacttcccagggggtgggggtagtgaatgccttgtgcccacacaggggcacactgccacctcttggagacttgaaatgactggtgggggggttggacaaggctttgagcccaatcacctcttggacaggaaagtaacccccactttatggccctgctgtaaaagcccagtcaaacctcatttgtccaaggaagatagacctcttggggcttcctaaggataggggtgttctatatttgggccctgcttctaagcattcagccagctttattaaaggaaattcataacaaaacttgaatttcctgcttcttaaatactaatagtgtgctggatctccattaaaaatgctgtcttgcacagtaggctatggtttctgtgggctctctacagctatgggacaactggattctgttttctgaagggcatgtgtcagctcagtactgactatagacctatgagttctctgaccccctaactcacctttttttttcttgcctcagatttggccgtattggacgcctggttaccagggctgccttcacttctggcaaagtggaagttgttgccatcaatgaccccttcattgacctcaactacatggtctacatgttccagtatgactctacccatggcaagttcaaaggcacagtcaaggctgagaatggaaagcttgtcatcaacgggaaggccatcaccatcttccaggagcgagatcccgccaacatcaaatggggtgatgctggcgccgagtatgttgtggaatctactggcgtcttcaccaccatggagaaggctggggcccacttgaagggcggggccaagagggtcatcatctccgccccttctgctgatgcccccatgtttgtgatgggtgtgaaccaagacaagtatgacaactccctcaagattgtcaggtgaggatggcagagggctgtggcaaagtgggcaagcaggggcaaggttacaggtgggcgagcctcctaacctgtctcttctcttcagcaatgcgtcctgcaccaccaactgcttagcccccctggccaaggtcatccatgacaactttggcattgtggaaggactcatggtatgtagttcatctgtttcatcctgccagcagtgggcgctgtggtgggggccctgcaagacctcactccctgcctctgtgtctttcagaccacggtccatgccatcactgccacccagaagactgtggatggcccctccgggaagctgtggcgtgatggccgtggggctgcccagaacatcacccctgcatccactggcgctgccaaggctgtgggcaaagtcagcccagagctgaacgggaagctgactggcatggccttccgtgttcctacccccaacgtgtccgttgtggatctgacatgtcgcctggagaaacctgtatgtctggggtgggctgagggttgtctctagtggtgaggttggggcttgagtagtcaccttgatttttgcccttaataggccaagtatgaggacatcaagaaggtggtgaagcaggcatctgagggcccactgaagggcatcctgggctacaccgaggaccaggttgtctcctgcgacttcaacagtgactcccactcttccacctttgatgctggggctggcattgctctcaatgacaactttgtaaagctcatttcctggtatgtgggatcagaaacagtcttttaaaagtaacttggggtttgtcaccccttggtgtctaactcaaaaagtaaacccttcctaactgccctcccaatgtgttctaggtatgacaatgaatttggctacagcaacagagtggtggacctcatggcctacatggcctccaaggagtaagaagcccaccctggaccatccaccccagcaaggactcgagcaagagggaggccctggctgctgagcagtccctgtccaataacccccacaccgatcatctccctcacagtgtccatcccagacccccagaataaggaggggcttagggagccctactctcttgaataccatcaataaagttcactgcacccatcttccttggcctttcaatgtaaggttgggggagggaggctgtgacctagcaaggttgggaatcctctgtgtcacttttcaaacagggcactagccacatgccagcccaggtttcctgtcctgaacagatgaaaattcacctaaggtgtcttggtgctgggaggagtgggggttgacaatgggaccagtagtatggtcttagaggcttgggctggactgcatcaagttccagggctgtgtgtgtgttatctgcaaacaaaggtcaattgtgtctggaggccttaggtaaaattggaaggatgcccaacatagtaaaaatgtatcagccaggggaagtgactacactgtatctaacctgaaacagctgagctgtaagccagcagctgtcactatgttcaggtgtggtccgctggttctggggtggtcacttgtatccagtttgttaggaagtgttgtcattgcttgttaggaagacaacacatctcaggctgggcagtggtagtgcgtgcctctaatgccagcattcccagcacggaagtggcagaggcaggaggacagcctggaataacaacccagggcagagcccccatctcggaaaagacaaaaaccagaaagtgcttaaaacattgacacaaaggtgctcaaatattccttcattgcttttagagattccactgtcagcttggcatggcctctagtgagacatcctgacttggtcccctgctttccaaggtcaggagaatgatagccacagaacgttccctcagctgatggctggagaaccggggtccctgagcccccaccctcacacccatgtgcaggagggagcttcacctttccctccgagcagtgtctgccttcaggacaccgctctcagcccagacactgagtcttctttgtgtgcatttccccggggaaaagctacacattccctgtacagcaggcaggagggcagctgtgagctcatggacacgggacagaaggccaggtaccctgcctccctgcggtaccccgcctcccttcgaggccttactaggaagcaaagccttctccaggtgtacgtgatgcggaaagtctgcagaaagtcggggactcccagatgagtctgctgcacagtgcctgggccagttagcaccagaacaaaagtccaagatagccaaggagaacaggcaggtcccacaatcacattccttacaaacggagaagggaggatcacaagtgttctggcagcgtttcaaagatagagaagtagcaaacagtcagagtgaagaaggcaggagtgtcccaatgtcatggggatatccaggaagggtggggccaagcctagctccagacacccagggagcccgggcgaggagacagccccagggccaggcttcatgcagtggcacaaaagacccccaccccggcctcggtgagctccagggagccaggcactggtccctatctcatggagctatcagatgagacatggcaatcggagtcctcagtttcgcttggcgctggcggcgggtcgctaagcgggaccgcagtgaaagcaggagactttctagaaaaaaacaccagatgtcaccttggggcaagcaggaagcccaagcaggcctgcatgctcgctctcctttccatggccttatcttctggggtgcagctggatgccaggcagggaagtgggtgggaagacttgtccccattcacctctatgaggaacagttcccctcttggaggctctgagaggctcagagatcaaaaatgtctcaagtcacagcatggagatgcttcctgagcccacagtgtgctgacaccagggccagcttcttgtccagctgtaaagggggtggggctgtctcactatgttcccaaggctgacccccaacacttggtacaaagcaatcctcttgctccagagtagctgggatgacaagcaaaggccactaaacctagtgaacatgctgcacactgtgcctagcttaatgtaattgggggagtgggttggggttttgttttgagacagggtctcagtatgcattcctggctggcctggaattcacagaggagagaccggcctggtacttgtggcaatctgtagggagacaccgtaaccacgcctcctaagagctggctcatcagggtgtggtcaaggtgatgtcagggtgacatgggagaggtgtaaagggataaggaacgcacatggtagctcttttccctgctgctttggcttgctctgctttcctgccggttggctacctaataaatatatcctgaccatcacgggctatgtattagtaattaatcctaatagccggtgtctccctacaacaatccacctgcctgtgacacctatgtgcttggatgacagatgtacaccaccacacctggcttactgtggttccttagattttacctgttgttgctggttgttttattttaagacaagtttttgctattgtagtccaggatagcctcaaactcacggcagtcttttgcttcagcctcccaaggctgggcttgcagacatgtaccatcatgcctgggttacacatttttaatatataatttatttttattctgtgttaattggtatttgcctgcatatatgtctgtgtaaaggtatcagaagccctggaactggagttacagacactcgtgtgggtgctgggaattgaacccaggccctctggaagagcatccagtgttcttaactaatgagccatctctcagcccccaggtttttttttttttttttttactctgtgtgtatgtgtgtgtgtgcacacgcacgcgcatgctcgaacatacaccagcacacagggtctcacacaggctagacaagtactctaccactgagctatattccccaaacccaaatttactcttacacaaggtcaaatacttaaatcatgacttagactttggataaaaacctgcccctgtatctcttcattcctagccaggtttcggctctcagacactggggcactggtaggagtcgtctgaccaccagggcagggagaggaagctatcaatctgggccacacctataggtgactccaaaggacagctccacttgcagatgtgacctggtcaccggccactgggctgggacgagggccaatcagagcaggtccttaccggtcccccgactgcgtgatcagggcaggtccttaccggtcccccgactgtgtgatgaggcggcggcaggtctccatctgcacgtccaagccccgtttcatgctgcacatctccatgtactcgtgcaggtggcggttcatgtcattcttggctgtggccagctccagctgcagaaaagtagggcagggcaggatgaggaggttagcaagcccaaggcagacaggggattagagcccagctgggccccggaggccaggcagccatggttaaaagctcggccttcaaaagaaagcaagcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacacacacacacacacacacacacacacttacttcctgaggaagactgcagggaacaaggctagatagaacccattattccaggagacaaaagtgaagcaaaaatggtctcttaaagcacaaagcagcactaccaacagtggtctcttccagcctatatatcaccctagtttacacattgaagggaaagatgtacattctagaacattttcaacaagtcagcccccaaattccagactcagcatgggagatcagagcagtagaggtagatggaggagatagaggggccagggccatggaggaaaactcccacctgtcatcagctttaccatccaataatgggcagctctacctcagtaacactgagagattaagggccatacagacaaaaggaacttttgccacaaagctaccaagtgaactttctttacctttctcctcaagtcattcttccaggaaggacagaagtcacacaacttagaaacaactttccatccatgtgcagtatacatatttttatgtgtgtggatacagatgtgcatgtgtgtggttacatggggaagcctgatgtcgatgcagggaatcttcctaggtggttcttatactctgagtctgtgtaacggcattggttgtcctggaacttgatctgtagaccagactggcctcaaactcacagagatctacctgtctctgcctcccgaatgctgggattaaaagcgtgtgcctcagccggacgttggtggcgcatgcctttaatcccagcactcgggaggcagaggcaggaggatctctgtgagttctagaccagcctggtctacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctagttccagggcagcctccaaagccacagagaaaccctgtctcaaaaaacaaaacaaaaataaataaataaataaataaaatcctgcgcctccactgcccggctcagaatatttttaaagacaaggattatgtagcccaggctggcctcaaacttgttatgtagctaagatgatggtggcctctgatcctcctgcctctacctcccaagtgttaggactataggctggcactaccacacccggtttcatgtgggctaggatcacactcaggcagggcttcatgcacattgggcaggcattctaccaactgagttacacagtcccagcccaggatgtctcaccagatcacggttacagctaggcacctgtggtctgaggtttgcaccacatggcagccccagtcactgagctaaggtgaaactcatacttgactaaaggcacggaatctgaaaggggtgggtacaacccaggaggaaaatcggcggaagcaataagaggcgtcaacaggcaatggtcacttttccccagacaagtctcagctggaagcagcacacgggctgggtatctgaaactcagaagtttcctgggttaaaaacaaaagccctggtttttcttaggatagtatgttcatttgatgtgtgctgggacactgagaacacaggaaaataacttccaaagtttgttctcaaaatgcagacagggtgaacctttacagggcttgcagggaggctgatacaagaggtctcctgcctcattcacccatcagagtccaagacacagtcctcacacagccccacccaaccaactgtcctccgacggtgacctcctccctacaaacctgcctgcagcccagaaggacagtcagagccccagctccatagccatttgtaaaggactccccaggctcaaaaacaaaagggagactcctccaggagctcacaggctgagccaaggaggcaggagtgctccggcacttagggtgtccgggtgataatgcacaatatgtatgatgtgtcaacctgccagatcctgtcctaagcgccacgtgtctatctgatcatcctaataacccagcctgcggggagagacatgcacatctccttctgacaaataaagaggcaggcccaagcttagcatggcagtacacatctgtaacccagcactcaacaggcagagcaggaggattgactgcctcctattccaggccagtttggctacacagaaagaccctgtactgatgctaagagagatggctcagagttaagagcacttcctgctcctggagaggacccaggtttgctttccagcacccatgtcaggctcacagtcgcctgtgaacaccagctccaggggatctgatgccctcttctggcctccaggggcacgcgcgtgtgcgctcacacacacacacacacacacacacacacacacacacacacacacacacacacagagtgcaaagctgaagtgcagctcacacccataatcctagcaaatggagacaaaaggaccaggagttcgaggccaacctagactacaagacaagtacttgtaaataaatacatatatacgaaaataaacaaaccaaaaataatgaaacaggcactcagatcaagggagccagtattggtggcacacgcctttaatcccagcactcaggaggcagaggcaggtggatctctgagtttgaggccagcctagtctacagaatgagatccaggactacacagagaaaccctgtctcaccctcctctcccctaaaaaaaattaagaaagaaaagaaagagccaggggctggtaagcaatggatcagcaggtaaaggcacaagccaccaagcccaacaacctaaatttgatccctgggacctacatggtagaaggagagaactcactgcaagctgtcctctgacctccacacacacaccatggcaggcacatgctctccccgccacatacatacatacatacatacatacatacatacatacatacatacatgtaaacagaaaagaaagtaggaagacaaaaggttcattctaagtggtgacagagccaagatttgaacccacatctatctgcgtctagatcctgaggtctcaagggcccattagagctaccaaaactggggtggggtgcactcctcctgtcaacaggtggaaggtcctggtcttgggctctggctgggaggaggccagccagcagcacacctccagcctggtttgtgagccctgggaacagagcccctgccaaaccccttac

SEQ ID NO: 30 - DNA, 3020 bp, Artificial
  amplicon
tacggccggcttcactgtacagtggcacatacttgtcaccacagtgtcatactggacattttcatgaccctaaggccacatcacccctgtggtgtcactcagttgtaatttcaccctatatagtgatttcaggttggctttttacttagaatataacatttaaattatgtttcttttttggttttgagttttgtttttcgagacagggtttctctgtattgctttggaggttgtgctggaacttgctctggagaccaggctggcttcgaactcacagagatcctcccacctctgcctcccgagtgctgggattaaaggcgtgcgccaccaatgccccagcatctttctgtttctctgtttttaaagagggagataatgtttccagtgctgtttttaacttttttcctgtaggctaccatagatgaatttgagcagaagcttcgggcatgtcataccagaggcatggatggaatagaggagcttgaaactggccaagaaggcagccagcaagccctgtctgccaagaaaccctctgctggtatgagagacaagcccacacggactccccacaaatcatccctgtcatttattggtttttgtttttaaaatcacatctccctgccatttctagtctcaaggcgacagcctctgacttctatagattcagacaatgactttgtcacacctaagcctcgacgcaccaaccgtcatcgtccaaacactcagcaacgaaagtccaagaagaaagccaaagttgtcttttcaagtgatgagtccagtgaggatggtatgactcgccctcccggcagctgcttgcttgccttggggtctccttggggcttgcctggttgtagagtcattcttcctggtaatgtgttccccactgtgcacgttggctcacctttgcagctctatgttttaagatccactttacaccatctttgtgactcagcttttgttcctataattctttgaacagaactttcagcggaaatgacggaagaagagactcccaagagaaccacccccatccgcagatcatctgcccgaagacacaggtcttaggagttgcctcacctattcccatccctgctgtgcagggtatcttaattgatttttaattcatttcctcccttgtaaaaatgttgcctccttgtttttaatatgtaaatggtctttgcattcaaggcctttctcctagagcccatctgactgctgctttcactttccaacaaacacttaaccaatcaccatgttttctaagaataaatgtttttatatacttttattctttgctggtctgtttcatcttccaccttagcccagattcaggcagcaatagagggttatcaaaccagagagaagatacaccagcccaggcatcctgaggctgtctctgggtcttcatgtttcctcattgattcatcttgcacaactgatgagactccactgtagagaattctcacttctcagcctgggggtactttctacccagagacctatttctgtcatagccttttgggacacataaagcttccttcctgcatagaacaccccacaacagtgtatcaggagtgtacaagttgacaaacaatgctctagacctacggttttcttcctctgtgtgcctcatcccagaagagatcatgactcccaggagtcagcctttactatggggtctgcaggggcgtccagcccctcagcggcaagccatgcccaccctccccaagtccttaatctgctgagtcacttggaacaggagacactgattctgctgtcatgacaacagcacattgccatagaaatgctccctacctcttacgtgtgtggtgggggaacagtaatgacaaaccataggcaggaggcaaaagggaagacggcacctcagaaacatgtgttaggttagggcagaactatggaggggctcctgagactctttgatgggaaagggttaatgctgctcctgaaacctctgttggaaggcagaaaagggacagggctgagtccccgcactgggaccatttccatcctctgcatcctgcccccggctcatggaaagcctgggcatgggccacacagctgtcagtcttggctctggggccccaaggaggtagggcaatcccagaatggcaaggagccaggactggatttggggtgcagcccagcctgctccctgccttttaagcaaaggttatcaccaggccagctaaacttagcaattaggctcttcagctaaaagagcagggggctggtctcaagttgcactgacctagcaaagaggccccaggatccccctgcccagcacctgtggctgagctcccaagcccttcccgagagctcaggatccaccctttccaccctccctactcttcagaggaggaaccccctttctccttcccacttgttggagggggctggggccaggctgttctggcttggggtataataccccctaccccttctactttcccctcctctcagacctcaccctgcctccacgagggcagccaagaaaggagagtccctggctgcagggccagtaggcacgtcccaggacggggagggacttccgccctcacgtccagctctccgccctggggctgcagtgggtgaaaggggcagtgtctcctagcctgggcggtgcaaccctcaggttccgaggaggaacgctctgggaggcttctttgcctcctccaacccaacccacaaccaggacattgtcctcaccccggggccccaacctagaccttaactgaggaacacagaggccagtttgtaagtctcaattatgcagggcatcccgacctgtggcgtagggagcgcccctccaggccgcttccctagcctcctcctggccctcacagcccaggcctctggcccaagaaatggaagtgggggtgggggatggaactgcgaatgcgaagggcccccgcaggaggcaaagtgacccctcccgggccttttctgctccgagacttgtttttgcctgtgtcactaccgaagaaccacggccggcctga

SEQ ID NO: 31 - DNA, 3537 bp, Artificial
  amplicon
tactcgcgattcactgtacagtggcacatacttgtcaccacagtgtcatactggacattttcatgaccctaaggccacatcacccctgtggtgtcactcagttgtaatttcaccctatatagtgatttcaggttggctttttacttagaatataacatttaaattatgtttcttttttggttttgagttttgtttttcgagacagggtttctctgtattgctttggaggttgtgctggaacttgctctggagaccaggctggcttcgaactcacagagatcctcccacctctgcctcccgagtgctgggattaaaggcgtgcgccaccaatgccccagcatctttctgtttctctgtttttaaagagggagataatgtttccagtgctgtttttaacttttttcctgtaggctaccatagatgaatttgagcagaagcttcgggcatgtcataccagaggcatggatggaatagaggagcttgaaactggccaagaaggcagccagcaagccctgtctgccaagaaaccctctgctggtatgagagacaagcccacacggactccccacaaatcatccctgtcatttattggtttttgtttttaaaatcacatctccctgccatttctagtctcaaggcgacagcctctgacttctatagattcagacaatgactttgtcacacctaagcctcgacgcaccaaccgtcatcgtccaaacactcagcaacgaaagtccaagaagaaagccaaagttgtcttttcaagtgatgagtccagtgaggatggtatgactcgccctcccggcagctgcttgcttgccttggggtctccttggggcttgcctggttgtagagtcattcttcctggtaatgtgttccccactgtgcacgttggctcacctttgcagctctatgttttaagatccactttacaccatctttgtgactcagcttttgttcctataattctttgaacagaactttcagcggaaatgacggaagaagagactcccaagagaaccacccccatccgcagatcatctgcccgaagacacaggtcttaggagttgcctcacctattcccatccctgctgtgcagggtatcttaattgatttttaattcatttcctcccttgtaaaaatgttgcctccttgtttttaatatgtaaatggtctttgcattcaaggcctttctcctagagcccatctgactgctgctttcactttccaacaaacacttaaccaatcaccatgttttctaagaataaatgtttttatatacttttattctttgctggtctgtttcatcttccaccttagcccagattcaggcagcaatagagggttatcaaaccagagagaagatacaccagcccaggcatcctgaggctgtctctgggtcttcatgtttcctcattgattcatcttgcacaactgatgagactccactgtagagaattctcacttctcagcctgggggtactttctacccagagacctatttctgtcatagccttttgggacacataaagcttccttcctgcatagaacaccccacaacagtgtatcaggagtgtacaagttgacaaacaatgctctagacctacggttttcttcctctgtgtgcctcatcccagaagagatcatgactcccaggagtcagcctttactatggggtctgcaggggcgtccagcccctcagcggcaagccatgcccaccctccccaagtccttaatctgctgagtcacttggaacaggagacactgattctgctgtcatgacaacagcacattgccatagaaatgctccctacctcttacgtgtgtggtgggggaacagtaatgacaaaccataggcaggaggcaaaagggaagacggcacctcagaaacatgtgttaggttagggcagaactatggaggggctcctgagactctttgatgggaaagggttaatgctgctcctgaaacctctgttggaaggcagaaaagggacagggctgagtccccgcactgggaccatttccatcctctgcatcctgcccccggctcatggaaagcctgggcatgggccacacagctgtcagtcttggctctggggccccaaggaggtagggcaatcccagaatggcaaggagccaggactggatttggggtgcagcccagcctgctccctgccttttaagcaaaggttatcaccaggccagctaaacttagcaattaggctcttcagctaaaagagcagggggctggtctcaagttgcactgacctagcaaagaggccccaggatccccctgcccagcacctgtggctgagctcccaagcccttcccgagagctcaggatccaccctttccaccctccctactcttcagaggaggaaccccctttctccttcccacttgttggagggggctggggccaggctgttctggcttggggtataataccccctaccccttctactttcccctcctctcagacctcaccctgcctccacgagggcagccaagaaaggagagtccctggctgcagggccagtaggcacgtcccaggacggggagggacttccgccctcacgtccagctctccgccctggggctgcagtgggtgaaaggggcagtgtctcctagcctgggcggtgcaaccctcaggttccgaggaggaacgctctgggaggcttctttgcctcctccaacccaacccacaaccaggacattgtcctcaccccggggccccaacctagaccttaactgaggaacacagaggccagtttgtaagtctcaattatgcagggcatcccgacctgtggcgtagggagcgcccctccaggccgcttccctagcctcctcctggccctcacagcccaggcctctggcccaagaaatggaagtgggggtgggggatggaactgcgaatgcgaagggcccccgcaggaggcaaagtgacccctcccgggccttttctgctccgagacttgtttttgcctgtgtcactaccgaagaaccacgagaagatcctcaacttttccacagcctttgcataaaggggagagggtcggcggtgcagctgtggcacacacgcacttctgctcaacccgcccccccccgcccccgttcctgttccttcccaggttctccccattttatcggggcggcaacttttaggtccctgggtcctggaagtccttagtacacactcttcgtccttaagtccatagtctgtattccctcggtcctatcctgtcccccatcaccgggtcacctccccagcgaagcaatctcagttcccctccccctctcagccccgagcccacacgtttggtgcgtgcacatttcaaaaacgaggcgggtccaaagagagggggtggggaggtgccgagtggcccagctactcgcggctttacgggtgcacgtagctcaggcctcagcgcccttgagctgtgactggatggatgagcggggcgggaggcggggcgagcgtcctcggcgctccccaccaccccagttcctataaatagcagagctcgtttagtgaac

SEQ ID NO: 32 - DNA, 537 bp, Artificial
  amplicon
tactcgcgaagaagatcctcaacttttccacagcctttgcataaaggggagagggtcggcggtgcagctgtggcacacacgcacttctgctcaacccgcccccccccgcccccgttcctgttccttcccaggttctccccattttatcggggcggcaacttttaggtccctgggtcctggaagtccttagtacacactcttcgtccttaagtccatagtctgtattccctcggtcctatcctgtcccccatcaccgggtcacctccccagcgaagcaatctcagttcccctccccctctcagccccgagcccacacgtttggtgcgtgcacatttcaaaaacgaggcgggtccaaagagagggggtggggaggtgccgagtggcccagctactcgcggctttacgggtgcacgtagctcaggcctcagcgcccttgagctgtgactggatggatgagcggggcgggaggcggggcgagcgtcctcggcgctccccaccaccccagttcctataaatagcagagctcgtttagtgaac

SEQ ID NO: 33 - DNA, 30 bp, Artificial
  Primer GlnPr1896
tacggccggcttcactgtacagtggcacat

SEQ ID NO: 34 - DNA, 30 bp, Artificial
  Primer GlnPr1897
tcaggccggccgtggttcttcggtagtgac

SEQ ID NO: 35 - DNA, 35 bp, Artificial
  Primer GlnPr1901
tactcgcgaagaagatcctcaacttttccacagcc

SEQ ID NO: 36 - DNA, 40 bp, Artificial
  Primer GlnPr1902
gttcactaaacgagctctgctatttataggaactggggtg

SEQ ID NO: 37 - DNA, 40 bp, Artificial
  Primer GLnPr1903
caccccagttcctataaatagcagagctcgtttagtgaac

SEQ ID NO: 38 - DNA, 20 bp, Artificial
  Primer GlnPr1904
cgctagcaccggtcgatcga

SEQ ID NO: 39 - DNA, 31 bp, Artificial
  Primer GlnPr1905
tactcgcgattcactgtacagtggcacatac

(19) United States
(12) Patent Application Publication
(10) Pub. No.: US 2020/0087679 A1
(43) Pub. Date: Mar. 19, 2020

(54) EXPRESSION CASSETTE

(71) Applicant: GLENMARK PHARMACEUTICALS S.A., La Chaux-de-Fonds (CH)

(72) Inventors: Christel AEBISCHER-GUMY, La Chaux-de-Fonds (CH); Martin BERTSCHINGER, La Chaux-de-Fonds (CH); Daniel LUESCHER, La Chaux-de-Fonds (CH); Pierre MORETTI, La Chaux-de-Fonds (CH)

(21) Appl. No.: 16/397,006

(22) Filed: Apr. 29, 2019

Related U.S. Application Data
(63) Continuation of application No. 15/653,243, filed on Jul. 18, 2017, now abandoned.
Application No. 15/653,243 is a continuation of application No. 14/003,410, filed on Sep. 5, 2013, now abandoned.
(60) Provisional application No. 61/567,675, filed on Dec. 7, 2011.

(51) Int. Cl. C12N 15/85; C12N 15/79

(52) U.S. Cl. CPC C12N 15/85; C12N 15/79

(57) ABSTRACT

The present invention relates to an expression cassette useful for the expression of a polynucleotide sequence encoding a polypeptide.

### RELATED APPLICATION

This application a continuation of U.S. patent application Ser. No. 15/653,243, filed Jul. 18, 2017, which is a continuation of U.S. patent application Ser. No. 14/003,410, filed Sep. 5, 2013, which is the National Stage of International Application Number PCT/IB2012/056977, filed Dec. 5, 2012, which claims the benefit of U.S. Provisional Application No. 61/567,675, filed Dec. 7, 2011, each of which is incorporated by reference herein in its entirety.

### REFERENCE TO SEQUENCE LISTING SUBMITTED ELECTRONICALLY VIA EFS-WEB

The content of the electronically submitted sequence listing (Name: 3305_0060003_SeqListing.txt; Size: 148,870 bytes; and Date of Creation: Apr. 24, 2019) is herein incorporated by reference in its entirety.

### FIELD OF THE INVENTION

The present invention relates to an expression cassette useful for the expression of a polynucleotide sequence encoding a polypeptide. The present invention is also directed to vectors and host cells which comprise the expression cassette and uses of the expression cassette for the production of a polypeptide from a host cell.

### BACKGROUND OF THE INVENTION

Expression systems for the production of recombinant polypeptides are well-known in the state of the art and are described by, e.g., Marino M H (1989) Biopharm, 2: 18-33; Goeddel D V et al., (1990) Methods Enzymol 185: 3-7; Wurm F & Bernard A (1999) Curr Opin Biotechnol 10: 156-159. Polypeptides for use in pharmaceutical applications are preferably produced in mammalian cells such as CHO cells, NSO cells, SP2/0 cells, COS cells, HEK cells, BHK cells, or the like. The essential elements of an expression vector used for this purpose are normally selected from a prokaryotic plasmid propagation unit, for example E. coli, comprising a prokaryotic origin of replication and a prokaryotic selection marker, optionally a eukaryotic selection marker, and one or more expression cassettes for the expression of the structural gene(s) of interest each comprising a promoter, a polynucleotide sequence encoding a polypeptide, and optionally a transcription terminator including a polyadenylation signal. For transient expression in mammalian cells a mammalian origin of replication, such as the SV40 Ori or OriP, can be included. As promoter a constitutive or inducible promoter can be selected. For optimized transcription a Kozak sequence may be included in the 5′ untranslated region. For mRNA processing, in particular mRNA splicing and transcription termination, mRNA splicing signals, depending on the organization of the structural gene (exon/intron organization), may be included as well as a polyadenylation signal. Expression of a gene is performed either in transient or using a stable cell line. The level of stable and high expression of a polypeptide in a production cell line is crucial to the overall process of the production of recombinant polypeptides. The demand for biologic molecules such as proteins and specifically antibodies or antibody fragments has increased significantly over the last few years. High cost and poor yield have been limiting factors in the availability of biologic molecules and it has been a major challenge to develop robust processes that increase the yield of desirable biological molecules on an industrial scale. Thus there is still a need for improving the efficiency of expression vectors to obtain high expression in recombinant polypeptide production.

### SUMMARY OF THE INVENTION

The present invention relates generally to expression systems such as expression cassettes and expression vectors which can be used to obtain increased expression in recombinant polypeptide production. In one aspect, the present disclosure provides an expression cassette which comprises a promoter, a polynucleotide sequence encoding a polypeptide, and a non-translated genomic DNA sequence downstream of a eukaryotic Glyceraldehyde 3-phosphate dehydrogenase (GAPDH) promoter, wherein the polypeptide encoded by the polynucleotide sequence is not GAPDH, and wherein the non-translated genomic DNA sequence downstream of the eukaryotic GAPDH promoter starts within a region spanning from nucleotide position around +1 to nucleotide position around +7000, wherein the nucleotide position is relative to the transcription start of the GAPDH mRNA, and wherein the length of the non-translated genomic DNA sequence downstream of the eukaryotic GAPDH promoter is from around 100 to around 15000 nucleotides.

In a further aspect, the present disclosure provides an expression cassette which comprises a promoter, a polynucleotide sequence encoding a polypeptide, and a non-translated genomic DNA sequence upstream of a eukaryotic GAPDH promoter, wherein the polypeptide encoded by the polynucleotide sequence is not GAPDH, and wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, wherein the length of the non-translated genomic DNA sequence upstream of the eukaryotic GAPDH promoter is from 100 to around 15000 nucleotides, with the proviso that the expression cassette does not comprise a eukaryotic GAPDH promoter or fragments thereof.

In a further aspect, the present disclosure provides an expression vector comprising an expression cassette and a host cell comprising an expression cassette or an expression vector comprising an expression cassette.

In still further aspects, the present disclosure provides an in vitro method for the expression of a polypeptide, comprising transfecting a host cell with an expression cassette or an expression vector and recovering the polypeptide and the use of an expression cassette or an expression vector for the expression of a heterologous polypeptide from a mammalian host cell.

### BRIEF DESCRIPTION OF THE FIGURES

FIG. 1 shows reporter expression construct (REP) consisting of mouse cytomegalovirus promoter (mCMV), Ig donor acceptor fragment (IgDA) containing the first intron, IgG1 antibody light chain (IgG1 LC), Internal Ribosomal Entry Sites derived from Encephalomvocarditis virus (IRES), IgG1 antibody heavy chain (IgG1 HC), green fluorescent protein (GFP) and simian virus 40 polyadenylation signal (poly (A)).

FIG. 2 shows transient expression of IgG1 antibody in CHO-S cells on day 5 post-transfection (Mean of IgG titers are plotted for two independent transfections). Cells were transfected using the GAPDH_A and GAPDH_B vectors (GAPDH_A and GAPDH_B), the same vectors without GAPDH upstream and downstream elements (A and B) and the pGLEX41 vector as a control (pGLEX41). The concentration of the accumulated IgG1 antibody in the supernatant was determined using the Octet instrument (Fortebio, Menlo, Calif., USA).

FIG. 3 shows expression of IgG antibody in HEK293 EBNA cells. Cells were transfected using the GAPDH_A and GAPDH_B vectors (GAPDH_A and GAPDH_B) and the pGLEX41 vector as a control (pGLEX41). The supernatant was harvested and analysed on day 10 after transfection using the Octet instrument. The data represent N=3 independent transfections in tubespins per vector.

FIG. 4 shows an expression level study on a batch production using cellular pools. Cells were transfected and pools of stable cells were created using GAPDH_A and GAPDH_B vectors (GAPDH_A(1), GAPDH_A(2). GAPDH_B(1) and GAPDH_B(2)), the same vectors without the GAPDH upstream and downstream elements (A(1) and A(2)) and the pGLEX41 vector as a control (pGLEX41). After 7 days of culture the supernatant was analyzed using the Octet instrument for accumulated antibody in the supernatant. Mean of IgG titers are given (μg/ml) for each pool. The data represent N=2 batches per pool.

FIG. 5 shows an expression level study on populations generated by stable transfection and limiting dilution. Cells were transfected using the GAPDH_A and GAPDH_B vectors (GAPDH_A and GAPDH_B), the same vectors without the GAPDH upstream and downstream elements (A and B) and the pGLEX41 vector as a control (pGLEX41). The mean value of GFP fluorescence expressed by clones and minipools from stable transfections was read 14 days after transfection. Cells were cultivated under selection pressure in 96-well plates. The data represent N=48 clones or minipools per vector.

FIG. 6 shows the effect of medium additives insulin and PMA (phorbol 12-myristate 13-acetate, a phorbol ester) on expression of IgG1 antibody in the supernatant. After transfection with the GAPDH_A vector (GAPDH_A) and the pGLEX41 vector as a control (pGLEX41) the cells were either diluted in PowerCHO2 medium, 4 mM Gln, +/−insulin and PowerCHO2, 4 mM Gln, PMA+/−insulin. No difference in expression could be observed compared to the standard medium for pGLEX41 (filled bars) or GAPDH_A (open bars).

FIG. 7 shows an overview of the human GAPDH locus. The GAPDH gene is flanked by the genes NCAPD2 and IFFO1.

FIG. 8 shows details of the human GAPDH gene, the GAPDH up- and downstream elements and the fragments created for the analysis of the GAPDH upstream fragmentation study. The NruI restriction site was introduced to facilitate cloning steps and is not part of the genomic 5′ GAPDH upstream sequence (it is therefore highlighted using an asterisk). The sizes of the fragments are: Fragment 1 (SEQ ID NO: 9): 511 bps, Fragment 2 (SEQ ID NO: 10): 2653 bps, Fragment 3 (SEQ ID NO: 11): 1966 bps, Fragment 4 (SEQ ID NO: 12): 1198 bps, Fragment 8 (SEQ ID NO: 13): 259 bps, Fragment 9 (SEQ ID NO: 14): 1947 bps, Fragment 11 (SEQ ID NO: 15): 1436 bps, and Fragment 17 (SEQ ID NO: 16): 1177 bps.

FIG. 9 shows expression results of fragmentation of the GAPDH upstream and downstream elements. Expression results were obtained in transient transfection in CHO cells on day 10 after transfection. The quantification was done using the Octet instrument. Vector pGLEX41 serves as negative control. pGLEX41-ampiA also is a negative control showing the basal expression of the vector without the GAPDH flanking elements. pGLEX41-up/down contains the full length flanking (upstream and downstream) regions and serves as positive control. pGLEX41-up contains only the upstream flanking region and pGLEX41-down contains only the downstream flanking region. All other constructs contain the fragments described in FIG. 8. The fragments 2 and 3 were either cloned in the same direction as IgG1 LC and IgG1 HC or in opposite direction in relation to IgG1 LC and IgG1 HC (AS).

FIG. 10 shows transient expression of IgG1 antibody in CHO-S cells on day 8 post-transfection (Mean of IgG titers are plotted for three independent transfections: error bars: SD+/−). Cells were transfected using vectors with the Chinese hamster GAPDH upstream element in combination with the mouse CMV (A_GAPDH_UP) or the Chinese hamster GAPDH promoter (A_GAPDH_UP_PR). The plasmids having only the mouse CMV (A) or the Chinese hamster GAPDH promoter (A_PR) were transfected as a control. The concentration of the accumulated IgG1 antibody in the supernatant was determined using the Octet QK instrument (Fortebio, Menlo, Calif., USA).

### DETAILED DESCRIPTION OF THE INVENTION

The present disclosure relates to expression cassettes and expression vectors which comprise a promoter, a polynucleotide sequence encoding a polypeptide, and a non-translated genomic DNA sequence downstream of a eukaryotic Glyceraldehyde 3-phosphate dehydrogenase (GAPDH) promoter, wherein the polypeptide encoded by the polynucleotide sequence is not GAPDH, and wherein the non-translated genomic DNA sequence downstream of the eukaryotic GAPDH promoter starts within a region spanning from nucleotide position around +1 to nucleotide position around +7000, wherein the nucleotide position is relative to the transcription start of the GAPDH mRNA, and wherein the length of the non-translated genomic DNA sequence downstream of the eukaryotic GAPDH promoter is from around 100 to around 15000 nucleotides.

The present disclosure further relates to an expression cassette which comprises a promoter, a polynucleotide sequence encoding a polypeptide, and a non-translated genomic DNA sequence upstream of a eukaryotic GAPDH promoter, wherein the polypeptide encoded by the polynucleotide sequence is not GAPDH, and wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, wherein the length of the non-translated genomic DNA sequence upstream of the eukaryotic GAPDH promoter is from 100 to around 15000 nucleotides, with the proviso that the expression cassette does not comprise a eukaryotic GAPDH promoter or fragments thereof.

The term “expression cassette” as used herein includes a polynucleotide sequence encoding a polypeptide to be expressed and sequences controlling its expression such as a promoter and optionally an enhancer sequence, including any combination of cis-acting transcriptional control elements. The sequences controlling the expression of the gene, i.e. its transcription and the translation of the transcription product, are commonly referred to as regulatory unit. Most parts of the regulatory unit are located upstream of coding sequence of the gene and are operably linked thereto. The expression cassette may also contain a downstream 3′ untranslated region comprising a polyadenylation site. The regulatory unit of the invention is either operably linked to the gene to be expressed, i.e. transcription unit, or is separated therefrom by intervening DNA such as for example by the 5′-untranslated region of the heterologous gene. Preferably the expression cassette is flanked by one or more suitable restriction sites in order to enable the insertion of the expression cassette into a vector and/or its excision from a vector. Thus, the expression cassette according to the present invention can be used for the construction of an expression vector, in particular a mammalian expression vector. The expression cassette of the present invention may comprise one or more e.g. two, three or even more non-translated genomic DNA sequences downstream of a eukaryotic GAPDH promoter or fragments thereof, and/or one or more e.g. two, three or even more non-translated genomic DNA sequences upstream of a eukaryotic GAPDH promoter or fragments thereof. If the expression cassette of the present invention comprises more than one DNA sequence downstream and/or upstream of a eukaryotic GAPDH promoter or fragments thereof these DNA sequences may be directly linked, i.e. may comprise linker sequences e.g. linker sequences containing restriction sites that are attached to the 5′- and 3′-ends and that allow comfortable sequential cloning of the sequences or fragments thereof. Alternatively, the DNA sequences downstream and/or upstream of a eukaryotic GAPDH promoter or fragments thereof may be not directly linked, i.e. may be cloned with intervening DNA sequences.

The term “polynucleotide sequence encoding a polypeptide” as used herein includes DNA coding for a gene, preferably a heterologous gene expressing the polypeptide.

The terms “heterologous coding sequence”, “heterologous gene sequence”, “heterologous gene”, “recombinant gene” or “gene” are used interchangeably. These terms refer to a DNA sequence that codes for a recombinant, in particular a recombinant heterologous protein product that is sought to be expressed in a host cell, preferably in a mammalian cell and harvested. The product of the gene can be a polypeptide. The heterologous gene sequence is naturally not present in the host cell and is derived from an organism of the same or a different species and may be genetically modified.

The terms “protein” and “polypeptide” are used interchangeably to include a series of amino acid residues connected to the other by peptide bonds between the alpha-amino and carboxy groups of adjacent residues.

The term “non-translated genomic DNA sequence” as used herein includes DNA that constitutes genetic information of an organism. The genome of almost all organisms is DNA, the only exceptions being some viruses that have a RNA genome. Genomic DNA molecules in most organisms are organized into DNA-protein complexes called chromosomes. The size, number of chromosomes, and nature of genomic DNA varies between different organisms. Viral DNA genomes can be single- or double-stranded, linear or circular. All other organisms have double-stranded DNA genomes. Bacteria have a single, circular chromosome. In eukaryotes, most genomic DNA is located within the nucleus (nuclear DNA) as multiple linear chromosomes of different sizes. Eukaryotic cells additionally contain genomic DNA in the mitochondria and, in plants and lower eukaryotes, the chloroplasts. This DNA is usually a circular molecule and is present as multiple copies within these organelles. A non-translated genomic DNA sequence is normally not operably linked to a promoter and thus is not translated. It may contain gene(s) which are not translated, thus gene(s) that encode e.g. a protein which is not expressed.

The term “non-translated genomic DNA sequence downstream of a eukaryotic GAPDH promoter” as used herein corresponds to non-translated eukaryotic genomic DNA 3′ of a eukaryotic GAPDH promoter. Non-translated genomic DNA sequence downstream of a eukaryotic GAPDH promoter normally starts at nucleotide position around +1, preferably at nucleotide position +1, wherein the nucleotide position is relative to the transcription start of the GAPDH mRNA i.e. is relative to the origin of the transcription start of the eukaryotic gene coding for GAPDH. The non-translated genomic DNA sequence downstream of a eukaryotic GAPDH promoter is usually of the same origin as the eukaryotic GAPDH promoter, e.g. if the GAPDH promoter is of human origin the non-translated genomic DNA sequence downstream of the human GAPDH promoter is as well of human origin and corresponds to the naturally occurring human genomic DNA sequence downstream of the human GAPDH promoter.

The term “non-translated genomic DNA sequence upstream of a eukaryotic GAPDH promoter” as used herein corresponds to non-translated eukaryotic genomic DNA 5′ of a eukaryotic GAPDH promoter. Non-translated genomic DNA sequence upstream of a eukaryotic GAPDH promoter normally starts at a nucleotide position around the 5′ end of the eukaryotic GAPDH promoter, preferably at the nucleotide position immediately after the 5′ end of the eukaryotic GAPDH promoter. The non-translated genomic DNA sequence upstream of a eukaryotic GAPDH promoter is usually of the same origin as the eukaryotic GAPDH promoter, e.g. if the GAPDH promoter is of human origin the non-translated genomic DNA sequence upstream of the human GAPDH promoter is as well of human origin and corresponds to the naturally occurring human genomic DNA sequence upstream of the human GAPDH promoter.

Positions of the eukaryotic GAPDH promoter, the non-translated genomic DNA sequence downstream or upstream of the eukaryotic GAPDH promoter and other DNA sequences as indicated herein are relative to the transcription start of the GAPDH mRNA e.g. are relative to the origin of the transcription start of the eukaryotic GAPDH if not specifically otherwise indicated.

The term “non-translated genomic DNA sequence upstream of a eukaryotic GAPDH promoter extends to” or “non-translated genomic DNA sequence downstream of a eukaryotic GAPDH promoter extends to” is used to define extension of the length of non-translated genomic DNA sequence upstream and/or downstream of a eukaryotic GAPDH promoter from the start to a particular genetic element e.g. extension to an intron. This extension includes the full length of the DNA sequence encoding the genetic element e.g. the intron or a part thereof.

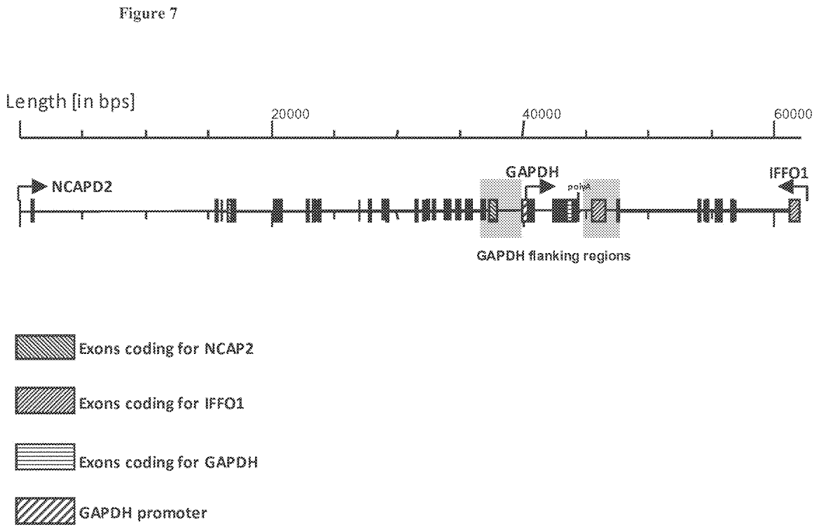

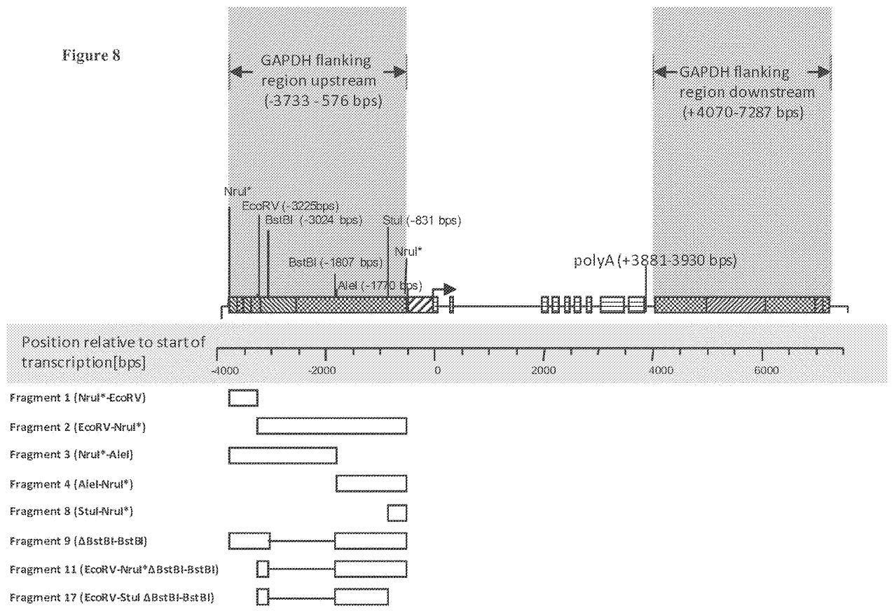

The eukaryotic GAPDH promoter and the eukaryotic genomic DNA upstream and/or downstream of the GAPDH promoter can be found for human, rat and mouse in the NCBI public databank (Entries for human, mouse, rat and Chinese hamster GAPDH gene are Gene IDs 2597 (mRNA: NM_002046.3), 14433 (mRMA: NM_008084.2), 24383 (mRNA: NM_017008.3) and 100736557 (mRNA: NM_001244854.2), respectively; National Center for Biotechnology Information (NCBI): http://www.ncbi.nlm.nih.gov/) and are exemplarily shown in FIGS. 7 and 8 for the human GAPDH gene.

The eukaryotic GAPDH promoter is usually considered to stretch from around bps −500 to around +50 relative to the transcription start of the GAPDH mRNA. The human GAPDH promoter is located on chromosome 12. The human GAPDH promoter is considered by Graven et al. (Graven et al., (1999) Biochimica et Biophysics Acta, 147: 203-218) to stretch from bps −488 to +20 relative to the transcription start of the GAPDH mRNA based on a fragmentation study. According to the NCBI public databank the human GAPDH promoter stretches from bps −462 to +46 relative to the transcription start of the GAPDH mRNA as defined by the NCBI public databank. If not specifically otherwise indicated, the human GAPDH promoter as referred to herein stretches from −462 to position +46 relative to the transcription start of the GAPDH mRNA which correspond to the sequence stretching from bps 4071 to 4578 of SEQ ID NO: 17.

The numbering used for the DNA of the GAPDH gene, the IFF01 gene and the NCAPD2 gene of human, mouse and rat origin as referred herein corresponds to the numbering used for these genes in the NCBI public databank (http://www.ncbi.nlm.nih.gov/).

The term “promoter” as used herein defines a regulatory DNA sequence generally located upstream of a gene that mediates the initiation of transcription by directing RNA polymerase to bind to DNA and initiating RNA synthesis.

The term “enhancer” as used herein defines a nucleotide sequence that acts to potentiate the transcription of genes independent of the identity of the gene, the position of the sequence in relation to the gene, or the orientation of the sequence. The vectors of the present invention optionally include enhancers.

The terms “functionally linked” and “operably linked” are used interchangeably and refer to a functional relationship between two or more DNA segments, in particular gene sequences to be expressed and those sequences controlling their expression. For example, a promoter and/or enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Orientation” refers to the order of nucleotides in a given DNA sequence. For example, an orientation of a DNA sequence in opposite direction in relation to another DNA sequence is one in which the 5′ to 3′ order of the sequence in relation to another sequence is reversed when compared to a point of reference in the DNA from which the sequence was obtained. Such reference points can include the direction of transcription of other specified DNA sequences in the source DNA and/or the origin of replication of replicable vectors containing the sequence.

The term “expression vector” as used herein includes an isolated and purified DNA molecule which upon transfection into an appropriate host cell provides for a high-level expression of a recombinant gene product within the host cell. In addition to the DNA sequence coding for the recombinant or gene product the expression vector comprises regulatory DNA sequences that are required for an efficient transcription of the DNA coding sequence into mRNA and for an efficient translation of the mRNAs into proteins in the host cell line.

The terms “host cell” or “host cell line” as used herein include any cells, in particular mammalian cells, which are capable of growing in culture and expressing a desired recombinant product protein.

The term “fragment” as used herein includes a portion of the respective nucleotide sequence e.g. a portion of the non-translated genomic DNA sequence downstream and/or upstream of a eukaryotic GAPDH promoter or a portion of the nucleotide sequence encoding a particular genetic element such as a promoter. Fragments of a non-translated genomic DNA sequence downstream and/or upstream of a eukaryotic GAPDH promoter may retain biological activity and hence alter e.g. increase the expression patterns of coding sequences operably linked to a promoter. Fragments of a non-translated genomic DNA sequence downstream and/or upstream of a eukaryotic GAPDH promoter may range from at least about 100 to about 3000 bp, preferably from about 200 to about 2800 bp, more preferably from about 300 to about 2000 bp nucleotides, in particular from about 500 to about 1500 bp nucleotides. In order to clone the fragments of the non-translated genomic DNA sequence downstream and/or upstream of a eukaryotic GAPDH promoter in the expression cassette of the present invention, usually linker sequences containing restriction sites that allow comfortable cloning are attached to the 5′- and 3′-ends of the fragments.

The term “nucleotide sequence identity” or “identical nucleotide sequence” as used herein include the percentage of nucleotides in the candidate sequence that are identical with the nucleotide sequence of e.g. the non-translated genomic DNA sequence downstream and/or upstream of a eukaryotic GAPDH promoter, after aligning the sequences and introducing gaps, if necessary, to achieve the maximum percent sequence identity. Thus sequence identity can be determined by standard methods that are commonly used to compare the similarity in position of the nucleotides of two nucleotide sequences. Usually the nucleotide sequence identity of the candidate sequence to the non-translated genomic DNA sequence downstream and/or upstream of a eukaryotic GAPDH promoter is at least 80%, preferably at least 85%, more preferably at least 90%, and most preferably at least 95%, in particular 96%, more particular 97%, even more particular 98%, most particular 99%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%.

The term “CpG site” as used herein include regions of DNA where a cytosine nucleotide occurs next to a guanine nucleotide in the linear sequence of bases along its length. “CpG” is shorthand for “-C-phosphate-G-”, that is, cytosine and guanine separated by only one phosphate; phosphate links any two nucleosides together in DNA. The “CpG” notation is used to distinguish this linear sequence from the CG base-pairing of cytosine and guanine.

The term “alternative codon usage” as used herein includes usage of alternative codons coding for the same amino acid in order to avoid the CpG sequence motif. This includes using preferably codons not having an internal CpG site (for example GCG coding for Alanine and containing a CpG site, might be replaced by either GCT, GCC or GCA) as well as avoiding joining of two codons that leads to a new CpG site.

The term “around” as used herein in relation to the length of a DNA sequence and in relation to a nucleotide position which is relative to the transcription start of the GAPDH mRNA e.g. is relative to the origin of the transcription start of the eukaryotic GAPDH includes values with deviations of a maximum of ±50%, usually of a maximum of ±10% of the stated values e.g. “around 3000 nucleotides” includes values of 2700 to 3300 nucleotides, preferably 2900 to 3100 nucleotides, more preferably 2995 to 3005 nucleotides, “around 100 nucleotides” includes values of 50 to 150 nucleotides, preferably 90 to 110 nucleotides, more preferably 95 to 105 nucleotides, “around 15000 nucleotides” includes values of 13500 to 16500 nucleotides, preferably 14500 to 15500 nucleotides, more preferably 14990 to 15010 nucleotides, most preferably 14995 to 15005 nucleotides. “around 200 nucleotides” includes values of 150 to 250 nucleotides, preferably 190 to 210 nucleotides, more preferably 195 to 205 nucleotides, “around 8000 nucleotides” includes values of 7200 to 8800, preferably 7500 to 8500 nucleotides, more preferably 7990 to 8010 nucleotides, most preferably 7995 to 8005 nucleotides, “around 500 nucleotides” includes values of 450 to 550 nucleotides, preferably 475 to 525, more preferably 490 to 510, most preferably 495 to 505 nucleotides, “around 5000 nucleotides” includes values of 4500 to 5500 nucleotides, preferably 4750 to 5250, more preferably 4990 to 5010, most preferably 4995 to 5005 nucleotides, “around 1000 nucleotides” includes values of 900 to 1100 nucleotides, preferably 950 to 1050, more preferably 990 to 1010, most preferably 995 to 1005 nucleotides, “around 4500 nucleotides” includes values of 4050 to 4950 nucleotides, preferably 4250 to 4750, more preferably 4490 to 4510, most preferably 4495 to 4505 nucleotides, “around 1500 nucleotides” includes values of 1350 to 1650 nucleotides, preferably 1450 to 1550, more preferably 1490 to 1510, most preferably 1495 to 1505 nucleotides. “around 4000 nucleotides” includes values of 3600 to 4400 nucleotides, preferably 3800 to 4200, more preferably 3990 to 4010, more preferably 3995 to 4005 nucleotides, “around 2000 nucleotides” includes values of 1800 to 2200 nucleotides, preferably 1900 to 2100, more preferably 1990 to 2010, most preferably 1995 to 2005 nucleotides, “around 3500 nucleotides” includes values of 3150 to 3850 nucleotides, preferably 3300 to 3700, more preferably 3490 to 3510, most preferably 3495 to 3505 nucleotides, “around 2700 nucleotides” includes values of 2430 to 2970 nucleotides, preferably 2600 to 2800, more preferably 2690 to 2710, most preferably 2695 to 2705 nucleotides, “around 3300 nucleotides” includes values of 2970 to 3630 nucleotides preferably 3100 to 3500, more preferably 3290 to 3310, most preferably 3295 to 3305 nucleotides, “around 3200 nucleotides” includes values of 2880 to 3520 nucleotides, preferably 3000 to 3400, more preferably 3190 to 3210, most preferably 3195 to 3205 nucleotides, around +7000 or around position +7000 includes positions +6300 to +7700, preferably positions +6700 to +7300, more preferably positions +6990 to +7010, most preferably positions +6995 to +7005, around +1 or around position +1 includes positions −10 to +10, preferably positions −5 to +5, more preferably positions −1 to +2, around −3500 or around position −3500 includes positions −3150 to −3850, preferably positions −3300 to −3700, more preferably positions −3490 to −5010, most preferably positions −3495 to −3505.

The term “around” as used herein in relation to the numbering used for the DNA of the GAPDH gene, the IFF01 gene and the NCAPD2 gene of human, mouse and rat origin as referred herein or used herein in relation to a position in a sequence of a SEQ ID number includes values with deviations of a maximum of ±500 bps, preferably ±100 bps, more preferably ±10 bps, most preferably ±5 bps.

In one embodiment, the present disclosure provides an expression cassette which comprises a promoter, a polynucleotide sequence encoding a polypeptide, and a non-translated genomic DNA sequence downstream of a eukaryotic GAPDH promoter, wherein the polypeptide encoded by the polynucleotide sequence is not GAPDH, and wherein the non-translated genomic DNA sequence downstream of the eukaryotic GAPDH promoter starts within a region spanning from nucleotide position around +1 to nucleotide position around +7000, wherein the nucleotide position is relative to the transcription start of the GAPDH mRNA, and wherein the length of the non-translated genomic DNA sequence downstream of the eukaryotic GAPDH promoter is from around 100 to around 15000 nucleotides.

In one embodiment, the length of the non-translated genomic DNA sequence downstream of a eukaryotic GAPDH promoter is at least around 100 nucleotides and extends at its maximum to the second last intron of the IFF01 gene or to a part thereof. In one embodiment, the length of the non-translated genomic DNA sequence downstream of a eukaryotic GAPDH promoter is at least around 100 nucleotides and extends at its maximum to the last intron of the IFF01 gene.

The human IFF01 gene is located in human DNA around bps 6665249 to 6648694 of chromosome 12 (NCBI gene ID: 25900). In one embodiment, the length of the non-translated genomic DNA sequence downstream of a eukaryotic GAPDH promoter extending at its maximum to the last intron of the IFF01 gene in human stretches at its maximum to around bps 6650677 of chromosome 12 coding for the IFF01 gene in human (position +7021). In one embodiment, the length of the non-translated genomic DNA sequence downstream of a eukaryotic GAPDH promoter extending at its maximum to the second last intron of the IFF01 gene in human stretches at its maximum to around bps 6657230 of chromosome 12 coding for the IFF01 gene in human (position +13574). The non-translated genomic DNA sequences downstream of a eukaryotic GAPDH promoter extending at its maximum to the last intron of the IFF01 gene in human and to the second last intron of the IFF01 gene in human, respectively, are included in SEQ ID NO: 17 which shows bps 6657230 to 6639125 of chromosome 12 (NCBI gene ID: 25900). The non-translated genomic DNA sequence downstream of a eukaryotic GAPDH promoter extending to the last intron stretches to around bps 11553 of the nucleotide sequence as shown by SEQ ID NO: 17 and the non-translated genomic DNA sequence downstream of a eukaryotic GAPDH promoter extending to the second last intron stretches to around bps 18106 of the nucleotide sequence as shown by SEQ ID NO: 17.

The mouse IFF01 gene (NCBI gene ID: 320678) is located in mouse DNA around bps 125095259 to 125111800 of chromosome 6. In one embodiment, the length of the non-translated genomic DNA sequence downstream of a eukaryotic GAPDH promoter extending at its maximum to the last intron of the IFF01 gene in mouse stretches at its maximum to around bps 125109211 of chromosome 6 coding for the IFF01 gene in mouse (position +6391). In one embodiment, the length of the non-translated genomic DNA sequence downstream of a eukaryotic GAPDH promoter extending at its maximum to the second last intron of the IFF01 gene in mouse stretches at its maximum to around bps 125103521 of chromosome 6 coding for the IFF01 gene in mouse (position +12081). The non-translated genomic DNA sequences downstream of a eukaryotic GAPDH promoter extending at its maximum to the last intron and to the second last intron of the IFF01 gene in mouse, respectively are included in SEQ ID NO: 18 which shows bps 125103521 to 125119832 of chromosome 6 (NCBI gene ID: 320678). The non-translated genomic DNA sequence downstream of a eukaryotic GAPDH promoter extending to the last intron of the IFFO1 gene in mouse stretches to around bps 10622 of the nucleotide sequence as shown by SEQ ID NO: 18 and the non-translated genomic DNA sequence downstream of a eukaryotic GAPDH promoter extending to the second last intron of the IFF01 gene in mouse stretches to around bps 16312 of the nucleotide sequence as shown by SEQ ID NO: 18.

The rat IFF01 gene (NCBI gene ID: 362437) is located in rat DNA around bps 161264966 to 161282150 of chromosome 4. In one embodiment, the length of the non-translated genomic DNA sequence downstream of a eukaryotic GAPDH promoter extending at its maximum to the last intron of the IFF01 gene in rat stretches at its maximum to around bps 161280937 of the chromosome 4 coding for IFF01 gene in rat (position +5154). In one embodiment, the length of the non-translated genomic DNA sequence downstream of a eukaryotic GAPDH promoter extending at its maximum to the second last intron of the IFF01 gene in rat stretches at its maximum to around bps 161279451 of chromosome 4 coding for the IFF01 gene in rat (position +6640).

The non-translated genomic DNA sequences downstream of a eukaryotic GAPDH promoter extending at its maximum to the last intron and to the second last intron of the IFF01 gene in rat, respectively are included in SEQ ID NO: 19 which shows bps 161279451 to 161290508 of chromosome 4 (NCBI gene ID: 362437). The non-translated genomic DNA sequence downstream of a eukaryotic GAPDH promoter extending to the last intron of the IFFO1 gene stretches to around bps 9572 of the nucleotide sequence as shown by SEQ ID NO: 19 and the non-translated genomic DNA sequence downstream of a eukaryotic GAPDH promoter extending to the second last intron of the IFF01 gene stretches to around bps 11058 bps of the nucleotide sequence as shown by SEQ ID NO: 19.

The Chinese hamster IFF01 gene (NCBI gene ID: 100753382) is located in Chinese hamster DNA around bps 3577293 to 3593683. In one embodiment, the length of the non-translated genomic DNA sequence downstream of a eukaryotic GAPDH promoter extending at its maximum to the last intron of the IFF01 gene in Chinese hamster stretches at its maximum to around bps 3579014 coding for IFF01 gene in Chinese hamster (position +6883). In one embodiment, the length of the non-translated genomic DNA sequence downstream of a eukaryotic GAPDH promoter extending at its maximum to the second last intron of the IFF01 gene in Chinese hamster stretches at its maximum to around bps 3585061 coding for the IFF01 gene in Chinese hamster (position +12930). The chromosomal location is not yet annotated in the NCBI databank and the current sequence information contains many unknown bases. Therefore the precise annotation of the limits may change with the availability of more accurate sequence information.

The non-translated genomic DNA sequences downstream of a eukaryotic GAPDH promoter extending at its maximum to the last intron and to the second last intron of the IFF01 gene in Chinese hamster, respectively are included in SEQ ID NO: 29 which shows bps 3567932 to 3585061. The non-translated genomic DNA sequence downstream of a eukaryotic GAPDH promoter extending to the last intron of the IFF01 gene stretches to around bps 11083 of the nucleotide sequence as shown by SEQ ID NO: 29 and the non-translated genomic DNA sequence downstream of a eukaryotic GAPDH promoter extending to the second last intron of the IFFO1 gene stretches to around bps 17130 bps of the nucleotide sequence as shown by SEQ ID NO: 29.

In a further embodiment, the non-translated genomic DNA sequence downstream of a eukaryotic GAPDH promoter starts at the eukaryotic GAPDH polyadenylation site e.g. starts at the first nucleotide encoding the eukaryotic GAPDH polyadenylation site. Preferably the non-translated genomic DNA sequence downstream of the eukaryotic GAPDH promoter starts downstream of the eukaryotic GAPDH polyadenylation site e.g. starts immediately after the last nucleotide encoding the eukaryotic GAPDH polyadenylation site. Even more preferred the non-translated genomic DNA sequence downstream of the eukaryotic GAPDH promoter starts downstream of the eukaryotic GAPDH polyadenylation site and the length of the non-translated genomic DNA sequence downstream of the eukaryotic GAPDH promoter is at least around 100 nucleotides and extends at its maximum to the second last intron of the IFF01 gene.

In one embodiment, the non-translated genomic DNA sequence downstream of the eukaryotic GAPDH promoter starts within a region spanning from nucleotide position around +3881 to nucleotide position around +5000, preferably within a region spanning from nucleotide position around +3931 to nucleotide position around +5000, more preferably within a region spanning from nucleotide position around +4070 to nucleotide position around +5000, wherein the nucleotide position is relative to the transcription start of the GAPDH mRNA. A non-translated genomic DNA sequence downstream of the eukaryotic GAPDH promoter which starts e.g. downstream of the eukaryotic GAPDH polyadenylation site used in the present invention usually starts at a nucleotide position around position +3931, preferably at a nucleotide position around +4070, wherein the nucleotide position is relative to the transcription start of the GAPDH mRNA.

In human the non-translated genomic DNA sequence downstream of the human GAPDH polyadenylation site starts at around nucleotide position +3931 (relative to the transcription start of the GAPDH mRNA which corresponds to bp 8463 as shown in SEQ ID NO: 17). Preferably, if the non-translated genomic DNA sequence downstream of the GAPDH polyadenylation site is from human, the non-translated genomic DNA sequence downstream of the GAPDH polyadenylation site starts at around +3931 (relative to the transcription start of the GAPDH mRNA: which corresponds to bp 8463 as shown in SEQ ID NO: 17) and its length is around 3357 bps corresponding to the sequence from around bps 8463 to around 11819 as shown in SEQ ID NO: 17, more preferably it starts at around +4070 (relative to the transcription start of the GAPDH mRNA which corresponds to bp 8602 as shown in SEQ ID NO: 17) and its length is around 3218 bps corresponding to the sequence from around bps 8602 to around 11819 as shown in SEQ ID NO: 17.

In a further embodiment, the non-translated genomic DNA sequence downstream of a eukaryotic GAPDH promoter comprises the nucleotide sequence selected from the group consisting of SEQ ID NOs: 8 and 21 or fragments thereof.

In a further embodiment, the non-translated genomic DNA sequence downstream of the eukaryotic GAPDH promoter comprises a nucleotide sequence complementary to the nucleotide sequence selected from the group consisting of SEQ ID NOs: 8 and 21 or fragments thereof.

In a further embodiment, the non-translated genomic DNA sequence downstream of the eukaryotic GAPDH promoter comprises a nucleotide sequence at least 80% identical to the nucleotide sequence selected from the group consisting of SEQ ID NOs: 8 and 21 or fragments thereof.

In some embodiments, the nucleotide sequence selected from the group consisting of SEQ ID NOs: 8 and 21 or fragments thereof, comprises five or less, preferably four or less, more preferably three or less, most preferred two or less, in particular one nucleic acid modification, wherein the nucleic acid modification(s) are preferably a nucleic acid substitution.

In a further embodiment, the length of the non-translated genomic DNA sequence downstream of the eukaryotic GAPDH promoter is preferably from around 200 to around 8000 nucleotides, more preferably from around 500 to around 5000 nucleotides, even more preferably from around 1000) to around 4500 nucleotides, most preferably from around 1500 to around 4000 nucleotides, in particular from around 2000 to around 3500 nucleotides, more particular from around 2700 to around 3300, even more particular around 3200, most particular 3218 nucleotides. The length of the non-translated genomic DNA sequence downstream of the eukaryotic GAPDH promoter as defined herein does not include any linker sequences added to the non-translated genomic DNA sequence.

In a further embodiment, the non-translated genomic DNA sequence downstream of the eukaryotic GAPDH promoter is orientated in the same direction as the polynucleotide sequence encoding a polypeptide.

In a further embodiment, the non-translated genomic DNA sequence downstream of the eukaryotic GAPDH promoter is orientated in opposite direction in relation to the polynucleotide sequence encoding a polypeptide.

In some embodiments, the expression cassette which comprises a promoter, a polynucleotide sequence encoding a polypeptide, and a non-translated genomic DNA sequence downstream of a eukaryotic GAPDH promoter further comprises a non-translated genomic DNA sequence upstream of a eukaryotic GAPDH promoter, wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, and wherein the length of the non-translated genomic DNA sequence upstream of the eukaryotic GAPDH promoter is from around 100 to around 15000 nucleotides.

In a another embodiment, the expression cassette comprises a promoter, a polynucleotide sequence encoding a polypeptide, and a non-translated genomic DNA sequence upstream of a eukaryotic GAPDH promoter, wherein the polypeptide encoded by the polynucleotide sequence is not GAPDH, and wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, wherein the length of the non-translated genomic DNA sequence upstream of the eukaryotic GAPDH promoter is from 100 to around 15000 nucleotides, with the proviso that the expression cassette does not comprise a eukaryotic GAPDH promoter or fragments thereof.

In some embodiments, the expression cassette further comprises a non-translated genomic DNA sequence downstream of a eukaryotic GAPDH promoter, wherein the non-translated genomic DNA sequence downstream of the eukaryotic GAPDH promoter starts within a region spanning from nucleotide position around +1 to nucleotide position around +7000, wherein the nucleotide position is relative to the transcription start of the GAPDH mRNA, and wherein the length of the non-translated genomic DNA sequence downstream of the eukaryotic GAPDH promoter is from around 100 to around 15000 nucleotides. In these embodiments the non-translated genomic DNA sequence downstream of a eukaryotic GAPDH promoter used is e.g. as described supra.

In some embodiments, the length of the non-translated genomic DNA sequence upstream of the eukaryotic GAPDH promoter is preferably from around 200 to around 8000 nucleotides, more preferably from around 500 to around 5000 nucleotides, even more preferably from around 1000 to around 4500 nucleotides, most preferably from around 1500 to around 4000 nucleotides, in particular from around 2000 to around 3500 nucleotides, more particular from around 2700 to around 3300, even more particular around 3200, most particular 3158 nucleotides in length. The length of the non-translated genomic DNA sequence upstream of the eukaryotic GAPDH promoter as defined herein does not include any linker sequences added to the non-translated genomic DNA sequence.

In a further embodiment, the length of the non-translated genomic DNA sequence upstream of the eukaryotic GAPDH promoter is at least around 100 nucleotides and extends at its maximum to the start codon of the NCAPD2 gene. In a further embodiment, the length of the non-translated genomic DNA sequence upstream of the eukaryotic GAPDH promoter is at least around 100 nucleotides and extends at its maximum to the third last intron of the NCAPD2 gene. In a further embodiment, the length of the non-translated genomic DNA sequence upstream of the eukaryotic GAPDH promoter is at least around 100 nucleotides and extends at its maximum to the second last intron of the NCAPD2 gene. In a further embodiment, the length of the non-translated genomic DNA sequence upstream of the eukaryotic GAPDH promoter is at least around 100 nucleotides and extends at its maximum to the last intron of the NCAPD2 gene.

The human NCAPD2 gene (NCBI gene ID: 9918) is located in human DNA around bps 6603298 to 6641132 of chromosome 12. In one embodiment, the length of the non-translated genomic DNA sequence upstream of a eukaryotic GAPDH promoter extending at its maximum to the last intron of the NCAPD2 gene in human stretches at its maximum to around 6640243 bps of chromosome 12 coding for the NCAPD2 gene in human (position −3414 relative to the transcription start of the GAPDH gene which corresponds to bp 1119 in SEQ ID NO: 17).

In one embodiment, the length of the non-translated genomic DNA sequence upstream of a eukaryotic GAPDH promoter extending at its maximum to the second last intron of the NCAPD2 gene in human stretches at its maximum to around 6639984 bps of chromosome 12 coding for the NCAPD2 gene in human (position −3673 relative to the transcription start of the GAPDH gene which corresponds to bp 860 in SEQ ID NO: 17).

In one embodiment, the length of the non-translated genomic DNA sequence upstream of a eukaryotic GAPDH promoter extending at its maximum to the third last intron of the NCAPD2 gene in human stretches at its maximum to around 6639125 bps of chromosome 12 coding for the NCAPD2 gene in human (position −4532 relative to the transcription start of the GAPDH gene; which corresponds to bp 1 in SEQ ID NO: 17).

The non-translated genomic DNA sequence upstream of a eukaryotic GAPDH promoter extending at its maximum to the last intron, to the second last intron and to the third last intron of the NCAPD2 gene in human, respectively are included in SEQ ID NO: 17, which shows bps 6657230 to 6639125 of chromosome 12 (NCBI gene ID: 9918).

The mouse NCAPD2 gene (Gene ID: 68298) is located in mouse DNA around position 125118025 to 125141604 of chromosome 6. In one embodiment, the length of the non-translated genomic DNA sequence upstream of a eukaryotic GAPDH promoter (estimated to have a a length of 500 bps upstream of the transcription start) extending at its maximum to the last intron of the NCAPD2 gene in mouse stretches at its maximum to around bps 125118607 of chromosome 6 coding for the NCAPD2 gene in mouse.

In one embodiment, the length of the non-translated genomic DNA sequence upstream of a eukaryotic GAPDH promoter extending at its maximum to the second last intron of the NCAPD2 gene in mouse stretches at its maximum to around 125118880 bps of chromosome 6 coding for the NCAPD2 gene in mouse.

In one embodiment, the length of the non-translated genomic DNA sequence upstream of a eukaryotic GAPDH promoter extending at its maximum to the third last intron of the NCAPD2 gene in mouse stretches at its maximum to around 125119832 bps of chromosome 6 coding for the NCAPD2 gene in mouse.

The non-translated genomic DNA sequence upstream of a eukaryotic GAPDH promoter extending at its maximum to the last intron, to the second last intron and to the third last intron of the NCAPD2 gene in mouse, respectively are included in SEQ ID NO: 18, which shows bps 125103521 to 125119832 of chromosome 6 (NCBI gene ID: 68298). The non-translated genomic DNA sequence upstream of a eukaryotic GAPDH promoter extending to the last intron stretches to around bps 1226 of the nucleotide sequence as shown by SEQ ID NO: 18 (−3006 relative to the transcription start of the mouse GAPDH mRNA). The non-translated genomic DNA sequence downstream of a eukaryotic GAPDH promoter extending to the second last intron stretches to around bps 953 of the nucleotide sequence as shown by SEQ ID NO: 18 (−3279 relative to the transcription start of the mouse GAPDH mRNA). The non-translated genomic DNA sequence downstream of a eukaryotic GAPDH promoter extending to the third last intron stretches to around bp 1 of the nucleotide sequence as shown by SEQ ID NO: 18 (−4231 relative to the transcription start of the mouse GAPDH mRNA).

The rat NCAPD2 gene (Gene ID: 362438) is located in eukaryotic DNA around position 161288671 to 161310417 of chromosome 4. In one embodiment, the length of the non-translated genomic DNA sequence upstream of a eukaryotic GAPDH promoter extending at its maximum to the last intron of the NCAPD2 gene in rat stretches at its maximum to around 161289191 bps of chromosome 4 coding for the NCAPD2 gene in rat. In one embodiment, the length of the non-translated genomic DNA sequence upstream of a eukaryotic GAPDH promoter extending at its maximum to the second last intron of the NCAPD2 gene in rat stretches at its maximum to around 161289446 bps of chromosome 4 coding for the NCAPD2 gene in rat. In one embodiment, the length of the non-translated genomic DNA sequence upstream of a eukaryotic GAPDH promoter extending at its maximum to the third last intron of the NCAPD2 gene in rat stretches at its maximum to around 161290508 bps of chromosome 4 coding for the NCAPD2 gene in rat.

The non-translated genomic DNA sequence upstream of a eukaryotic GAPDH promoter extending at its maximum to the last intron, to the second last intron and to the third last intron of the NCAPD2 gene in rat, respectively are included in SEQ ID NO: 19, which shows bps 161279451 to 161290508 of chromosome 4 (NCBI gene ID: 362438). The non-translated genomic DNA sequence upstream of a eukaryotic GAPDH promoter extending to the last intron stretches to around bps 1318 of the nucleotide sequence as shown by SEQ ID NO: 19 (−3101 relative to the transcription start of rat GAPDH mRNA). The non-translated genomic DNA sequence downstream of a eukaryotic GAPDH promoter extending to the second last intron stretches to around bps 1063 of the nucleotide sequence as shown by SEQ ID NO: 19 (position −3356 relative to the transcription start of rat GAPDH mRNA). The non-translated genomic DNA sequence downstream of a eukaryotic GAPDH promoter extending to the third last intron stretches to around bp 1 of the nucleotide sequence as shown by SEQ ID NO: 19 (position −4418 relative to the transcription start of rat GAPDH mRNA).

The Chinese hamster NCAPD2 gene (Gene ID: 100753087) is located in eukaryotic DNA around position 3544184 to 3569879. The chromosomal location is not available on the NCBI database. In one embodiment, the length of the non-translated genomic DNA sequence upstream of a eukaryotic GAPDH promoter extending at its maximum to the last intron of the NCAPD2 gene in Chinese hamster stretches at its maximum to around 3569380 bps in Chinese hamster. In one embodiment, the length of the non-translated genomic DNA sequence upstream of a eukaryotic GAPDH promoter extending at its maximum to the second last intron of the NCAPD2 gene in Chinese hamster stretches at its maximum to around 3569131 bps in Chinese hamster. In one embodiment, the length of the non-translated genomic DNA sequence upstream of a eukaryotic GAPDH promoter extending at its maximum to the third last intron of the NCAPD2 gene in Chinese hamster stretches at its maximum to around 3567932 bps in Chinese hamster.

The non-translated genomic DNA sequence upstream of a eukaryotic GAPDH promoter extending at its maximum to the last intron, to the second last intron and to the third last intron of the NCAPD2 gene in Chinese hamster, respectively are included in SEQ ID NO: 29, which shows bps 3567932 to 3585061. The non-translated genomic DNA sequence upstream of a eukaryotic GAPDH promoter extending to the last intron stretches to around bps 1449 of the nucleotide sequence as shown by SEQ ID NO: 29 (−2752 relative to the transcription start of Chinese hamster GAPDH mRNA). The non-translated genomic DNA sequence downstream of a eukaryotic GAPDH promoter extending to the second last intron stretches to around bps 1200 of the nucleotide sequence as shown by SEQ ID NO: 29 (position −3001 relative to the transcription start of Chinese hamster GAPDH mRNA). The non-translated genomic DNA sequence downstream of a eukaryotic GAPDH promoter extending to the third last intron stretches to around bp 1 of the nucleotide sequence as shown by SEQ ID NO: 29 (position −4200 relative to the transcription start of Chinese hamster GAPDH mRNA).

In some embodiments, the non-translated genomic DNA sequence upstream of a eukaryotic GAPDH promoter starts usually within a region spanning from nucleotide position around −500 to a nucleotide position around −3500, preferably within a region spanning from nucleotide position around −576 to nucleotide position around −3500, wherein the nucleotide position is relative to the transcription start of the GAPDH mRNA.

In some embodiments, the non-translated genomic DNA sequence upstream of the eukaryotic GAPDH promoter starts usually at a nucleotide position around position −500, preferably at a nucleotide position around −576, wherein the nucleotide position is relative to the transcription start of the GAPDH mRNA.

In human the non-translated genomic DNA sequence upstream of the human GAPDH promoter starts at around nucleotide position −463 (relative to the transcription start of the GAPDH mRNA which corresponds to bp 4533 as shown in SEQ ID NO: 17). Preferably, if the non-translated genomic DNA sequence upstream of the GAPDH promoter is from human, the non-translated genomic DNA sequence upstream of the GAPDH promoter starts at around −500 (relative to the transcription start of the GAPDH mRNA; which corresponds to bp 4533 as shown in SEQ ID NO: 17). More preferably, if the non-translated genomic DNA sequence upstream of the GAPDH promoter is from human, the non-translated genomic DNA sequence upstream of the GAPDH promoter starts at around −576 (relative to the transcription start of the GAPDH mRNA; which corresponds to bp 4533 as shown in SEQ ID NO: 17) and its length is around 3158 bps corresponding to the sequence from around bps 800 to around 3957 as shown in SEQ ID NO: 17.

In a further embodiment, the non-translated genomic DNA sequence upstream of the eukaryotic GAPDH promoter comprises a nucleotide sequence selected from the group consisting of SEQ ID NOs: 7, 9, 10, 11, 12, 13, 14, 15, 16, 20, 22, 23, 24, 25, 26, 27 and 28 or fragments thereof, preferably a nucleotide sequence selected from the group consisting of SEQ ID NOs: 7, 9, 10, 11, 12, 13, 14, 15 and 16 or fragments thereof, or a nucleotide sequence selected from the group consisting of SEQ ID NOs: 20, 22, 23, 24, 25, 26, 27, 28 and 16 or fragments thereof. More preferred is a nucleotide sequence selected from the group consisting of SEQ ID NOs: 10, 12, 15 and 16 or fragments thereof, more preferably a nucleotide sequence selected from the group consisting of SEQ ID NOs: 10, 12, 15 and 16 or fragments thereof, wherein nucleotide sequences comprising SEQ ID NOs: 10 and/or 16 are orientated in opposite direction in relation to the polynucleotide sequence encoding a polypeptide, and nucleotide sequences comprising SEQ ID NOs: 12 and/or 15 are orientated in the same direction as the polynucleotide sequence encoding a polypeptide. Equally more preferred is a nucleotide sequence selected from the group consisting of SEQ ID NOs: 23, 25, 28 and 16 or fragments thereof, more preferably a nucleotide sequence selected from the group consisting of SEQ ID NOs: 23, 25, 28 and 16 or fragments thereof, wherein nucleotide sequences comprising SEQ ID NOs: 23 and/or 16 are orientated in opposite direction in relation to the polynucleotide sequence encoding a polypeptide, and nucleotide sequences comprising SEQ ID NOs: 25 and/or 28 are orientated in the same direction as the polynucleotide sequence encoding a polypeptide.

In a further embodiment, the non-translated genomic DNA sequence upstream of the eukaryotic GAPDH promoter comprises a nucleotide sequence complementary to the nucleotide sequence selected from the group consisting of SEQ ID NOs: 7, 9, 10, 11, 12, 13, 14, 15, 16, 20, 22, 23, 24, 25, 26, 27 and 28 or fragments thereof, preferably a nucleotide sequence complementary to the nucleotide sequence selected from the group consisting of SEQ ID NOs: 7, 9, 10, 11, 12, 13, 14, 15 and 16 or fragments thereof, or a nucleotide sequence complementary to the nucleotide sequence selected from the group consisting of SEQ ID NOs: 20, 22, 23, 24, 25, 26, 27, 28 and 16 or fragments thereof. More preferred is a nucleotide sequence complementary to the nucleotide sequence selected from the group consisting of SEQ ID NOs: 10, 12, 15 and 16 or fragments thereof. Equally more preferred is a nucleotide sequence complementary to the nucleotide sequence selected from the group consisting of SEQ ID NOs: 23, 25, 28 and 16 or fragments thereof.

In a further embodiment, the non-translated genomic DNA sequence upstream of the eukaryotic GAPDH promoter comprises a nucleotide sequence at least 80% identical to the nucleotide sequence selected from the group consisting of SEQ ID NOs: 7, 9, 10, 11, 12, 13, 14, 15, 16, 20, 22, 23, 24, 25, 26, 27 and 28 or fragments thereof, preferably a nucleotide sequence at least 80% identical to the nucleotide sequence selected from the group consisting of SEQ ID NOs: 7, 9, 10, 11, 12, 13, 14, 15 and 16 or fragments thereof, or a nucleotide sequence at least 80% identical to the nucleotide sequence selected from the group consisting of SEQ ID NOs: 20, 22, 23, 24, 25, 26, 27, 28 and 16 or fragments thereof. More preferred is a nucleotide sequence at least 80% identical to the nucleotide sequence selected from the group consisting of SEQ ID NOs: 10, 12, 15 and 16 or fragments thereof, more preferably a nucleotide sequence at least 80% identical to the nucleotide sequence selected from the group consisting of SEQ ID NOs: 10, 12, 15 and 16 or fragments thereof, wherein nucleotide sequences comprising SEQ ID NOs: 10 and/or 16 are orientated in opposite direction in relation to the polynucleotide sequence encoding a polypeptide, and nucleotide sequences comprising SEQ ID NOs: 12 and/or 15 are orientated in the same direction as the polynucleotide sequence encoding a polypeptide. Equally more preferred is a nucleotide sequence at least 80% identical to the nucleotide sequence selected from the group consisting of SEQ ID NOs: 23, 25, 28 and 16 or fragments thereof, more preferably a nucleotide sequence at least 80% identical to the nucleotide sequence selected from the group consisting of SEQ ID NOs: 23, 25, 28 and 16 or fragments thereof, wherein nucleotide sequences comprising SEQ ID NOs: 23 and/or 16 are orientated in opposite direction in relation to the polynucleotide sequence encoding a polypeptide, and nucleotide sequences comprising SEQ ID NOs: 25 and/or 28 are orientated in the same direction as the polynucleotide sequence encoding a polypeptide.

In some embodiments, the nucleotide sequence selected from the group consisting of SEQ ID NOs: 7, 9, 10, 11, 12, 13, 14, 15, 16, 20, 22, 23, 24, 25, 26, 27 and 28 or fragments thereof, comprises five or less, preferably four or less, more preferably three or less, most preferred two or less, in particular one nucleic acid modification, wherein the nucleic acid modification(s) are preferably a nucleic acid substitution.

In some embodiments, the nucleotide sequence selected from the group consisting of SEQ ID NOs: 7, 9, 11, 14, 20, 22, 24 and 27 or fragments thereof, comprises five or less, preferably four or less, more preferably three or less, most preferred two or less, in particular one nucleic acid modification, wherein the nucleic acid modification(s) are preferably a nucleic acid substitution.

In some embodiments, the nucleotide sequence selected from the group consisting of SEQ ID NOs: 7, 9, 11, 14, or fragments thereof, comprises one nucleic acid substitution at position 16 relative to the start of the nucleotide sequence of SEQ ID NOs: 7, 9, 11, 14. Preferably G at position 16 relative to the start of the nucleotide sequence is replaced with T.

In some embodiments, the nucleotide sequence selected from the group consisting of SEQ ID NOs: 20, 22, 24 and 27 or fragments thereof, comprises one nucleic acid substitution at position 13 relative to the start of the nucleotide sequence of SEQ ID NOs: 20, 22, 24 and 27. Preferably G at position 13 relative to the start of the nucleotide sequence is replaced with T.

In a further embodiment, the non-translated genomic DNA sequence upstream of the eukaryotic GAPDH promoter is orientated in the same direction as the polynucleotide sequence encoding a polypeptide.

In a further embodiment, the non-translated genomic DNA sequence upstream of the eukaryotic GAPDH promoter is orientated in opposite direction in relation to the polynucleotide sequence encoding a polypeptide.

In a preferred embodiment, the expression cassette comprises a promoter, a polynucleotide sequence encoding a polypeptide, and a non-translated genomic DNA sequence downstream of a eukaryotic GAPDH promoter and a non-translated genomic DNA sequence upstream of a eukaryotic GAPDH promoter as described supra. Preferably the origin of the non-translated genomic DNA sequence downstream of a eukaryotic GAPDH promoter and the non-translated genomic DNA sequence upstream of a eukaryotic GAPDH promoter is the same i.e. is of the same species. More preferably the origin of the non-translated genomic DNA sequence downstream of a eukaryotic GAPDH promoter, the non-translated genomic DNA sequence upstream of a eukaryotic GAPDH promoter and the host cell is the same i.e. is of the same species, e.g. the origin of the non-translated genomic DNA sequence downstream of a eukaryotic GAPDH promoter, the non-translated genomic DNA sequence upstream of a eukaryotic GAPDH promoter and the host cell is from the same mammal e.g. from human.

In some embodiments, if the non-translated genomic DNA sequence downstream and/or upstream of the eukaryotic GAPDH promoter is non-translated genomic DNA sequence from one species, the promoter of the expression cassette is not a GAPDH promoter from the same species.

In some embodiments, if the non-translated genomic DNA sequence downstream and/or upstream of the eukaryotic GAPDH promoter is non-translated genomic DNA sequence downstream and/or upstream of human origin, the promoter of the expression cassette is not a human GAPDH promoter.

In some embodiments, the promoter of the expression cassette is not a GAPDH promoter. In one embodiment, if the expression cassette comprises a promoter, a polynucleotide sequence encoding a polypeptide, and a non-translated genomic DNA sequence downstream of a eukaryotic Glyceraldehyde 3-phosphate dehydrogenase (GAPDH) promoter, wherein the polypeptide encoded by the polynucleotide sequence is not GAPDH, and wherein the non-translated genomic DNA sequence downstream of the eukaryotic GAPDH promoter starts within a region spanning from nucleotide position around +1 to nucleotide position around +7000, wherein the nucleotide position is relative to the transcription start of the GAPDH mRNA, and wherein the length of the non-translated genomic DNA sequence downstream of the eukaryotic GAPDH promoter is from around 100 to around 15000 nucleotides and wherein the expression cassette further comprises a non-translated genomic DNA sequence upstream of a eukaryotic GAPDH promoter, wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, and wherein the length of the non-translated genomic DNA sequence upstream of the eukaryotic GAPDH promoter is from around 100 to around 15000 nucleotides, the promoter of the expression cassette may be a eukaryotic GAPDH promoter, preferably a mammalian GAPDH promoter, more preferably a rodent or human GAPDH promoter. In this embodiment the non-translated genomic DNA sequence upstream of the eukaryotic GAPDH promoter starting within a region spanning from around the 5′ end of the eukaryotic GAPDH promoter to nucleotide position around −3500 is preferably located directly upstream of the eukaryotic GAPDH promoter, more preferably in this embodiment the expression cassette comprises the naturally occurring genomic DNA sequence comprising the eukaryotic GAPDH promoter and extending to nucleotide position around −3500, wherein the nucleotide position is relative to the transcription start of the GAPDH mRNA.

In some embodiments, the non-translated genomic DNA sequence downstream and/or upstream of the eukaryotic GAPDH promoter is of mammalian origin, e.g. the eukaryotic GAPDH promoter is a mammalian GAPDH promoter and non-translated genomic DNA sequence downstream and/or upstream of the mammalian GAPDH promoter is used as described herein.

In some embodiments, the non-translated genomic DNA sequence downstream and/or upstream of the eukaryotic GAPDH promoter is of rodent or human origin, e.g. the eukaryotic GAPDH promoter is a rodent or human GAPDH promoter and non-translated genomic DNA sequence downstream and/or upstream of the rodent or the human GAPDH promoter is used as described herein.

Preferably the non-translated genomic DNA sequence downstream and/or upstream of the eukaryotic GAPDH promoter is selected from human, rat or mouse origin, more preferably from human or mouse origin, most preferably from human origin.

In some embodiments, the non-translated genomic DNA sequence downstream and/or upstream of the eukaryotic GAPDH promoter is not operably linked to the polynucleotide sequence encoding the polypeptide.

In some embodiments, the expression cassette comprises a polyadenylation site. Preferably the polyadenylation site is selected from the group consisting of SV40 poly(A) and BGH (Bovine Growth Hormone) poly(A).

In some embodiments, the promoter and the polynucleotide sequence encoding a polypeptide of the expression cassette are operably linked.

In some embodiments, the promoter of the expression cassette is selected from the group consisting of SV40 promoter, human tk promoter. MPSV promoter, mouse CMV, human CMV, rat CMV, human EFlalpha, Chinese hamster EFlalpha, human GAPDH, hybrid promoters including MYC, HYK and CX promoter.

In some embodiments, the polypeptide encoded by the expression cassette can be a non-glycosylated and glycosylated polypeptide. Glycosylated polypeptides refer to polypeptides having at least one oligosaccharide chain.

Examples for non-glycosylated proteins are e.g. non-glycosylated hormones; non-glycosylated enzymes; non-glycosylated growth factors of the nerve growth factor (NGF) family, of the epithelial growth factor (EGF) and of the fibroblast growth factor (FGF) family and non-glycosylated receptors for hormones and growth factors.

Examples for glycosylated proteins are hormones and hormone releasing factors, clotting factors, anti-clotting factors, receptors for hormones or growth factors, neurotrophic factors cytokines and their receptors, T-cell receptors, surface membrane proteins, transport proteins, homing receptors, addressins, regulatory proteins, antibodies, chimeric proteins, such as immunoadhesins, and fragments of any of the glycosylated proteins. Preferably the polypeptide is selected from the group consisting of antibodies, antibody fragments or antibody derivates (e.g. Fc fusion proteins and particular antibody formats like bispecific antibodies). Antibody fragment as used herein includes, but is not limited to, (i) a domain, (ii) the Fab fragment consisting of VL, VH, CL or CK and CHI domains, including Fab′ and Fab′-SH, (iii) the Fd fragment consisting of the VH and CHI domains, (iv) the dAb fragment (Ward E S et al., (1989) Nature, 341(6242): 544-6) which consists of a single variable domain (v) F(ab′)2 fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al., (1988) Science, 242(4877): 423-6; Huston J S et al., (1988) Proc Natl Acad Sci USA, 85(16): 5879-83), (vii) “diabodies” or “triabodies”, multivalent or multispecific fragments constructed by gene fusion (Holliger P et al., (1993) Proc Natl Acad Sci USA, 90(14): 6444-8; Holliger P et al., (2000) Methods Enzymol. 326: 461-79), (viii) scFv, diabody or domain antibody fused to an Fc region and (ix) scFv fused to the same or a different antibody.

In some embodiments the expression cassette further comprises a genetic element selected from the group consisting of an additional promoter, an enhancer, transcriptional control elements, and a selectable marker, preferably a selectable marker which is expressed in animal cells. Transcriptional control elements are e.g. Kozak sequences or transcription terminator elements.

In one embodiment, the genetic element is a selectable marker wherein the content of CpG sites contained in the polynucleotide sequence encoding the selectable marker is 45 or less, preferably 40 or less, more preferably 20 or less, in particular 10 or less, more particular 5 or less, most particular 0 (CpG sites have been completely removed).

In a further aspect, the present disclosure provides an expression vector, preferably a mammalian expression vector comprising an expression cassette as described supra. In some embodiments, the expression vector comprises at least two separate transcription units. An expression vector with two separate transcription units is also referred to as a double-gene vector. An example thereof is a vector, in which the first transcription unit encodes the heavy chain of an antibody or a fragment thereof and the second transcription unit encodes the light chain of an antibody. Another example is a double-gene vector, in which the two transcription units encode two different subunits of a protein such as an enzyme. However, it is also possible that the expression vector of the present invention comprises more than two separate transcription units, for example three, four or even more separate transcription units each of which comprises a different nucleotide sequence encoding a different polypeptide chain. An example therefore is a vector with four separate transcription units, each of which contains a different nucleotide sequence encoding one subunit of an enzyme consisting of four different subunits.

In some embodiments, the expression vector further comprises a genetic element selected from the group consisting of an additional promoter, an enhancer, transcriptional control elements, an origin of replication and a selectable marker.

In some embodiments, the expression vector further comprises an origin of replication and a selectable marker wherein the content of the CpG sites contained in the polynucleotide sequence of the expression vector encoding the origin of replication and the selectable marker is 200 or less, preferably 150 or less, in particular 100 or less, more particular 50 or less, most particular 30 or less.

Any selection marker commonly employed such as thymidine kinase (tk), dihydrofolate reductase (DHFR), puromycin, neomycin or glutamine synthetase (GS) may be used for the expression cassette or the expression vector of the present invention. Preferably, the expression vectors of the invention also comprise a limited number of useful restriction sites for insertion of the expression cassette for the secretion of a heterologous protein of the present invention. Where used in particular for transient/episomal expression only, the expression vectors of the invention may further comprise an origin of replication such as the oriP origin of Epstein Barr Virus (EBV) or SV40 virus for autonomous replication/episomal maintenance in eukaryotic host cells but may be devoid of a selectable marker. Transient expression in cell lacking relevant factors to facilitate replication of the vector is also possible. The expression vector harbouring the expression cassette may further comprise an expression cassette coding for a fluorescent marker, an expression cassette coding for an ncRNA, an expression cassette coding for an antiapoptotic protein, or an expression cassette coding for a protein increasing the capacity of the secretory pathway.

In a further aspect, the present disclosure provides an expression vector, which comprises in order:

a) a non-translated genomic DNA sequence upstream and/or downstream of a eukaryotic GAPDH promoter

b) a promoter

c) a polynucleotide sequence encoding a polypeptide

d) a polyadenylation site

e) an enhancer

f) a non-translated genomic DNA sequence downstream and/or upstream of a eukaryotic GAPDH promoter, or

a) a non-translated genomic DNA sequence upstream and/or downstream of a eukaryotic GAPDH promoter

b) an enhancer

c) a promoter

d) a polynucleotide sequence encoding a polypeptide

e) a polyadenylation site

f) a non-translated genomic DNA sequence downstream and/or upstream of a eukaryotic GAPDH promoter, or

a) an enhancer

b) a non-translated genomic DNA sequence upstream and/or downstream of a eukaryotic GAPDH

c) a promoter

d) a polynucleotide sequence encoding a polypeptide

e) a polyadenylation site

f) non-translated genomic DNA sequence downstream and/or upstream of a eukaryotic GAPDH,

wherein inclusion of the enhancer is optional, and wherein the polypeptide encoded by the polynucleotide sequence is not GAPDH, and wherein the non-translated genomic DNA sequence downstream of the eukaryotic GAPDH promoter starts within a region spanning from nucleotide position around +1 to nucleotide position around +7000, wherein the nucleotide position is relative to the transcription start of the GAPDH mRNA, and wherein the length of the non-translated genomic DNA sequence downstream of the eukaryotic GAPDH promoter is from around 100 to around 15000 nucleotides and wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, and wherein the length of the non-translated genomic DNA sequence upstream of the eukaryotic GAPDH promoter is from around 100 to around 15000 nucleotides, with the proviso that if a) or b) is a non-translated genomic DNA sequence upstream of a eukaryotic GAPDH f) is a non-translated genomic DNA sequence downstream of a eukaryotic GAPDH and if a) or b) is a non-translated genomic DNA sequence downstream of a eukaryotic GAPDH f) is a non-translated genomic DNA sequence upstream of a eukaryotic GAPDH.

In some embodiments, the present disclosure provides an expression vector, which comprises in order:

a) a non-translated genomic DNA sequence upstream of a eukaryotic GAPDH promoter

b) a promoter

c) a polynucleotide sequence encoding a polypeptide

d) a polyadenlyation site

e) an enhancer

f) a non-translated genomic DNA sequence downstream of a eukaryotic GAPDH promoter, wherein inclusion of the enhancer is optional.

In a further aspect, the present disclosure provides an expression vector, which comprises in order:

a) a non-translated genomic DNA sequence upstream of a eukaryotic GAPDH promoter

b) an enhancer

c) a promoter

d) a polynucleotide sequence encoding a polypeptide

e) a polyadenlyation site

f) a non-translated genomic DNA sequence downstream of a eukaryotic GAPDH, wherein inclusion of the enhancer is optional.

In a further aspect, the present disclosure provides an expression vector, which comprises in order:

a) an enhancer

b) a non-translated genomic DNA sequence upstream of a eukaryotic GAPDH

c) a promoter

d) a polynucleotide sequence encoding a polypeptide

e) a polyadenlyation site

f) non-translated genomic DNA sequence downstream of a eukaryotic GAPDH, wherein inclusion of the enhancer is optional.

In a further aspect, the present disclosure provides an expression vector, which comprises in order:

a) a non-translated genomic DNA sequence downstream of a eukaryotic GAPDH promoter

b) a promoter

c) a polynucleotide sequence encoding a polypeptide

d) a polyadenlyation site

e) an enhancer

f) a non-translated genomic DNA sequence upstream of a eukaryotic GAPDH promoter, wherein inclusion of the enhancer is optional.

In a further aspect, the present disclosure provides an expression vector, which comprises in order:

a) a non-translated genomic DNA sequence downstream of a eukaryotic GAPDH promoter

b) an enhancer

c) a promoter

d) a polynucleotide sequence encoding a polypeptide

e) a polyadenlyation site

f) a non-translated genomic DNA sequence upstream of a eukaryotic GAPDH, wherein inclusion of the enhancer is optional.

In a further aspect, the present disclosure provides an expression vector, which comprises in order:

a) an enhancer

b) a non-translated genomic DNA sequence downstream of a eukaryotic GAPDH

c) a promoter

d) a polynucleotide sequence encoding a polypeptide

e) a polyadenlyation site

f) non-translated genomic DNA sequence upstream of a eukaryotic GAPDH, wherein inclusion of the enhancer is optional.

Non-translated genomic DNA sequence upstream of a eukaryotic GAPDH, enhancer, promoter, polynucleotide sequence encoding a polypeptide, polyadenlyation site and non-translated genomic DNA sequence downstream of a eukaryotic GAPDH promoter of the expression vectors are e.g. as described supra.

In a further aspect, the present disclosure provides a host cell comprising an expression cassette or an expression vector as described supra. The host cell can be a human or non-human cell. Preferred host cells are mammalian cells. Preferred examples of mammalian host cells include, without being restricted to, Human embryonic kidney cells (Graham F L et al., J. Gen. Virol. 36: 59-74), MRC5 human fibroblasts, 983M human melanoma cells, MDCK canine kidney cells, RF cultured rat lung fibroblasts isolated from Sprague-Dawley rats, B16BL6 murine melanoma cells, P815 murine mastocytoma cells. MTI A2 murine mammary adenocarcinoma cells, PER:C6 cells (Leiden, Netherlands) and Chinese hamster ovary (CHO) cells or cell lines (Puck et al., 1958, J. Exp. Med. 108: 945-955).

In a particular preferred embodiment the host cell is a Chinese hamster ovary (CHO) cell or cell line. Suitable CHO cell lines include e.g. CHO-S(Invitrogen, Carlsbad, Calif., USA), CHO K1 (ATCC CCL-61), CHO pro3-, CHO DG44, CHO P12 or the dhfr-CHO cell line DUK-BII (Chasin et al., PNAS 77, 1980, 4216-4220), DUXBI 1 (Simonsen et al., PNAS 80, 1983, 2495-2499), or CHO-K1SV (Lonza, Basel, Switzerland).

In a further aspect, the present disclosure provides an in vitro method for the expression of a polypeptide, comprising transfecting a host cell with the expression cassette or an expression vector as described supra and recovering the polypeptide. The polypeptide is preferably a heterologous, more preferably a human polypeptide.

For transfecting the expression cassette or the expression vector into a host cell according to the present invention any transfection technique such as those well-known in the art, e.g. electoporation, calcium phosphate co-precipitation, DEAE-dextran transfection, lipofection, can be employed if appropriate for a given host cell type. It is to be noted that the host cell transfected with the expression cassette or the expression vector of the present invention is to be construed as being a transiently or stably transfected cell line. Thus, according to the present invention the present expression cassette or the expression vector can be maintained episomally i.e. transiently transfected or can be stably integrated in the genome of the host cell i.e. stably transfected.

A transient transfection is characterised by non-appliance of any selection pressure for a vector borne selection marker. In transient expression experiments which commonly last 2 to up to 10 days post transfection, the transfected expression cassette or expression vector are maintained as episomal elements and are not yet integrated into the genome. That is the transfected DNA does not usually integrate into the host cell genome. The host cells tend to lose the transfected DNA and overgrow transfected cells in the population upon culture of the transiently transfected cell pool. Therefore expression is strongest in the period immediately following transfection and decreases with time. Preferably, a transient transfectant according to the present invention is understood as a cell that is maintained in cell culture in the absence of selection pressure up to a time of 2 to 10 days post transfection.

In a preferred embodiment of the invention the host cell e.g. the CHO host cell is stably transfected with the expression cassette or the expression vector of the present invention. Stable transfection means that newly introduced foreign DNA such as vector DNA is becoming incorporated into genomic DNA, usually by random, non-homologous recombination events. The copy number of the vector DNA and concomitantly the amount of the gene product can be increased by selecting cell lines in which the vector sequences have been amplified after integration into the DNA of the host cell. Therefore, it is possible that such stable integration gives rise, upon exposure to further increases in selection pressure for gene amplification, to double minute chromosomes in CHO cells. Furthermore, a stable transfection may result in loss of vector sequence parts not directly related to expression of the recombinant gene product, such as e.g. bacterial copy number control regions rendered superfluous upon genomic integration. Therefore, a transfected host cell has integrated at least part or different parts of the expression cassette or the expression vector into the genome.

In a further aspect, the present disclosure provides the use of the expression cassette or an expression vector as described supra for the expression of a heterologous polypeptide from a mammalian host cell, in particular the use of the expression cassette or an expression vector as described supra for the in vitro expression of a heterologous polypeptide from a mammalian host cell.

Expression and recovering of the protein can be carried out according to methods known to the person skilled in the art.

For the expression of a polypeptide, the non-translated genomic DNA sequence downstream and/or upstream of a eukaryotic GAPDH promoter of the expression cassette or of the expression vector as described supra and the host cell as described supra are used and are usually of the same origin. Surprisingly it has been found that an increase of expression is obtained if the non-translated genomic DNA sequence downstream and/or upstream of a eukaryotic GAPDH promoter of the expression cassette or of the expression vector and the host cell are of different origin e.g. if human DNA sequences downstream and/or upstream of a eukaryotic GAPDH promoter are used in CHO cells.

In a further aspect, the present disclosure provides the use of the expression cassette or the expression vector as described supra for the preparation of a medicament for the treatment of a disorder.

In a further aspect, the present disclosure provides the expression cassette or the expression vector as described supra for use as a medicament for the treatment of a disorder.

In a further aspect, the present disclosure provides the expression cassette or the expression vector as described supra for use in gene therapy.

### EXAMPLES

### Example 1: Cloning of Expression Vectors

### I. Materials and Methods

### I.1 Plasmids Constructs

### I.1.1. LB Culture Plates

500 ml of water were mixed and boiled with 16 g of LB Agar (Invitrogen, Carlsbad, Calif., USA) (1 litre of LB contains 10 g tryptone, 5 g yeast extract and 10 g NaCl). After cooling down, the respective antibiotic was added to the solution which is then plated (ampicillin plates at 100 μg/ml and kanamycin plates at 50 μg/ml).

### I.1.2. Polymerase Chain Reaction (PCR)

All PCR were performed using 1 μl of dNTPs (10 mM for each dNTP; Invitrogen, Carlsbad, Calif., USA), 2 units of Phusion® DNA Polymerase (Finnzymes Oy, Espoo, Finland), 25 nmol of Primer A (Mycrosynth, Balgach, Switzerland), 25 nmol of Primer B (Mycrosynth, Balgach, Switzerland), 10 d of 5×HF buffer (7.5 mM MgCl₂, Finnzymes, Espoo, Finland), 1.5 μl of Dimethyl sulfoxide (DMSO, Finnzymes, Espoo. Finland) and 1-3 μl of the template (1-2 μg) in a 50 μl final volume. All primers used are listed in Table 1.

The PCR were started by an initial denaturation at 98° C. for 3 minutes, followed by 35 cycles of 30 sec denaturation at 98° C., 30 sec annealing at a primer-specific temperature (according to CG content) and 2 min elongation at 72° C. A final elongation at 72° C. for 10 min was performed before cooling and keeping at 4° C.

TABLE 1
Summary of primers used in PCRs. GAPDH:
Glyceraldehyde 3-phosphate dehydrogenase
sequence, 5′: upstream sequence, 3:
downstream sequence. The “T” (underlined)
in primer GlnPr1172 was introduced in order
to avoid the formation of primer dimers.

| | | Sequence | |
|---|---|---|---|
| Primer | Primer sequence | amplified | Seq ID |
| GlnPr | ATTATTCGCGATGGCTCCTGGCA | 5′ GAPDH | SEQ ID |
| 1171 | TCTCTGGGACCGAGGC | | No: 1 |
| GlnPr | ATCGTCGCGAAGCTTGAGATTGT | | SEQ ID |
| 1172 | CCAAGCAGGTAGCCAG | | No: 2 |
| GlnPr | AGCAAGTACTTCTGAGCCTTCA | 3′GAPDH | SEQ ID |
| 1173 | GTAATGGCTGCCTG | | No: 3 |
| GlnPr | TGGCAGTACTAAGCTGGCACCA | | SEQ ID |
| 1174 | CTACTTCAGAGAACAAG | | No: 4 |

### I.1.3. Restriction Digest

For all restriction digests around 1 μg of plasmid DNA (quantified with NanoDrop, ND-1000 Spectrophotometer (Thermo Scientific. Wilmington, Del., USA)) was mixed to 10-20 units of each enzyme, 4 μl of corresponding 10× NEBuffer (NEB, Ipswich, Mass., USA), and the volume was completed to 40 μl with sterile H₂O. Without further indication, digestions were incubated 1 h at 37° C.

After each preparative digestion of backbone, 1 unit of Calf Intestinal Alkaline Phosphatase (CIP; NEB, Ipswich, Mass., USA) was added and the mix was incubated 30 min at 37° C.

If the digest was done in NEBuffer 3 (NEB. Ipswich. Mass. USA), the buffer was changed to NEB buffer 4 before adding the CIP because this enzyme has a strong activity in this buffer and may also digest some of the nucleotides at the external ends.

### I.1.4. PCR Purification and Agarose Gel Electrophoresis

I.1.4.1. PCR Clean Up

To allow digestion all PCR fragments were cleaned prior to restriction digests using the Macherey Nagel Extract II kit (Macherey Nagel, Oensingen, Switzerland) following the manual of the manufacturer using 40 μl of elution buffer. This protocol was also used for changing buffers of DNA samples.

I.1.4.2. DNA Extraction

For gel electrophoresis, 1% gels were prepared using UltraPure™ Agarose (Invitrogen, Carlsbad, Calif., USA) and 50× Tris Acetic Acid EDTA buffer (TAE, pH 8.3; Bio RAD. Munich, Germany). For staining of DNA 1 μl of Gel Red Dye (Biotum, Hayward, Calif., USA) was added to 100 ml of agarose gel. As a size marker 2 μg of the 1 kb DNA ladder (NEB, Ipswich, Mass., USA) was used. The electrophoresis was run for around 1 hour at 125 Volts.

The bands of interests were cut out from the agarose gel and purified using the kit Extract II (Macherey-Nagel, Oensingen, Switzerland), following the manual of the manufacturer using 40 μl of elution buffer.

### I.1.5. Ligation

For each ligation, 4 μl of insert were mixed to 1 μl of vector, 400 units of ligase (T4 DNA ligase, NEB, Ipswich, Mass., USA), 1 μl of 10× ligase buffer (T4 DNA ligase buffer; NEB, Ipswich, Mass. USA) in a 10 μl volume. The mix was incubated for 1-2 h at RT.

I.1.6. Transformation of Ligation Products into Competent Bacteria

For the cloning of pGLEX41-[REP] and for constructs made with the pCR-Blunt vector which contain a standard origin of replication, TOP 10 (One Shot® TOP 10 Competent E. coli: Invitrogen, Carlsbad, Calif., USA) were used.

For replication initiation of plasmid containing the R6K origin of replication, the expression of the a protein, coded by the pir sequence, is required. The a protein is expressed by One Shot® PIR1 competent E. coli (Invitrogen, Carlsbad, Calif., USA). These bacteria were used for all vectors containing the R6K sequence.

To transform competent bacteria with the ligation product, 25-50 μl of bacteria were thawed on ice for 5 minutes. Then, 3-5 μl of ligation product were added to competent bacteria and incubated for 20-30 min on ice before the thermic shock for 1 minute at 42° C. Then, 500 μl of S.O.C medium (Invitrogen, Carlsbad, Calif. USA) were added per tube and incubated for 1 hour at 37° C. under agitation. Finally, the bacteria are put on a LB plate with ampicillin (Sigma-Aldrich, St. Louis, Mo., USA) and incubated overnight at 37° C. For the cloning in pCR-Blunt vectors, plates with kanamycin (Sigma-Aldrich. St. Louis, Mo., USA) were used.

### I.1.7. Plasmid Preparation in Small (Mini) and Medium Scale (Midi)

1.1.7.1. Minipreparation

For minipreparation, colonies of transformed bacteria were grown for 6-16 hours in 2.5 ml of LB and ampicillin or kanamycin at 37° C., 200 rpm. The DNA was extracted with a plasmid purification kit for E. coli (QuickPure, Macherey Nagel, Oensingen, Switzerland), following the provided manual.

Plasmid DNA from minipreparations was quantified once with the NanoDrop ND-1000 Spectrophotometer (Thermo Scientific, Wilmington, Del., USA) by measuring the absorbance at 260 nm and assessing the ratio of the OD260 nm/OD280 nm that had to be between 1.8 and 2. A control digestion was performed before sending the sample to Fasteris SA (Geneva, Switzerland) for sequence confirmation.

For BAC extraction, the QuickPure kit (Macherey Nagel, Oensingen, Switzerland) was used with the following modification of the protocol: 10 ml of LB and chloramphenicol (12.5 j±g/ml) (Sigma-Aldrich, St. Louis. Mo., USA) were seeded with bacteria containing pBACe3.6 vector. After incubation on a shaking platform at 37° C. over night, the culture was centrifuged for 5 min at 13 300 rpm before being resuspended in 500 μl of A1 Buffer, 500 μl of A2 Lysis Buffer were added and the solution was incubated 5 min at RT. Then, it was neutralized with 600 μl of A3 buffer and centrifuged 10 min at 13 300 rpm. The supernatant was loaded on a column and from this step onwards the standard protocol of QuickPure miniprep kit was used.

I.1.7.2. Midipreparation

For midipreparation, transformed bacteria were grown at 37° C. overnight in 200 to 400 ml of LB and ampicillin (or kanamycin). Then, the culture was centrifuged 20 min at 725 g and the plasmid was purified using a commercial kit (NucleoBond Xtra Midi: Macherey Nagel, Oensingen, Switzerland) following the low plasmid protocol provided in the manual of the manufacturer.

Plasmid-DNA from midipreparation was quantified three times with the NanoDrop ND-1000 Spectrophotometer, confirmed by restriction digest and finally sent for sequencing (Fasteris SA, Geneva, Switzerland).

### II. Results and Discussion

### II.1. Cloning of DNA Regions Upstream and Downstream of the GAPDH Expression Cassette (5′ and 3′GAPDH)

The BAC clone RPCIB753F11841Q was ordered at Imagene (Berlin, Germany). This clone contains the human GAPDH sequence in a pBACe3.6 vector backbone, containing a chloramphenicol resistance gene. After DNA extraction by minipreparation, the vector concentration was determined by Nanodrop to 27 ng/μl.

DNA sequences immediately surrounding the GAPDH expression cassette upstream of the promoter and downstream of the poly-adenylation site were amplified by PCR using 27 ng of the purified clone RPCIB753F1841Q as template. The 3 kb fragment upstream of the promoter was amplified with primers GlnPr1171 (SEQ ID NO: 1) and GlnPr1172 (SEQ ID NO: 2) leading to the amplicon with SEQ ID No. 5. As primer GInPr1172 (SEQ ID NO: 2) carries a base change (G to T) relative to the template sequence, all sequences derived from this PCR reaction will carry this base change, too. The change is located in position −3721 relative to the transcription start of the GAPDH gene (bp 812 of SEQ ID NO: 17, position 23 relative to the start of SEQ ID NO: 5). The 3 kb fragment downstream of the polyadenlyation site was amplified with primers GlnPr1173 (SEQ ID NO: 3) and GlnPr1174 (SEQ ID NO: 4) leading to the amplicon with SEQ ID NO: 6 (Table 1). The annealing temperature used for these PCRs was 72° C.

The 5′ and 3′GAPDH fragments (SEQ ID NOs: 5 and 6) were cloned in pCR-Blunt, a commercially available PCR-product cloning vector (pCR-Blunt. PCR Zero Blunt cloning kit. Invitrogen). The ligation products were transformed into TOP10 competent bacteria and plated on kanamycin LB-agar plates. Colonies were amplified and plasmids were isolated by minipreps. Control digests were performed to identify positives clones yielding pCR-Blunt-5′GAPDH and pCR-Blunt-3′GAPDH constructs.

### II.2. Preparation of the DNA Fragment Coding for the Reporter Proteins GFP and a Recombinant IgG1 Monoclonal Antibody (LC-IRES—HC-IRES-GFP)

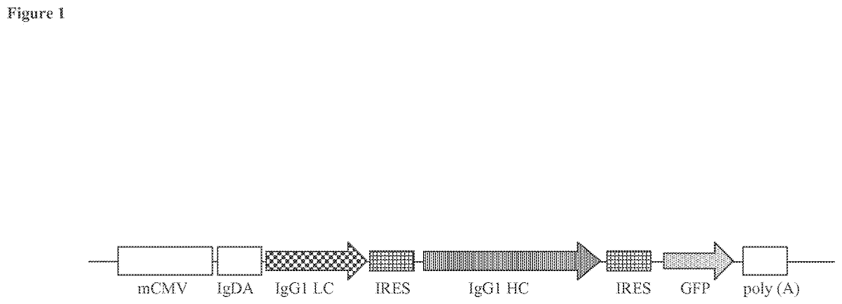

The reporter construct (REP) used in the present work consisted in a polycistronic gene: IgG1 monoclonal antibody light chain (LC)-IRES-IgG1 monoclonal antibody heavy chain(HC)-IRES-green fluorescent protein (GFP). The presence of Internal Ribosomal Entry Sites (IRES) derived from Encephalomyocarditis virus (Gurtu et al., Biochem Biophys Res Commun.; 229(1): 295-298, 1996)) allows the translation of the 3 peptides IgG1 monoclonal antibody light chain (LC), IgG1 monoclonal antibody heavy chain (HC) and GFP (FIG. 1). Transfected cells will therefore secrete the IgG1 monoclonal antibody and accumulate intracellular GFP in a dependent manner. However, polycistronic mRNAs are not common in eukaryotic cells and their translation is not very efficient, leading to relative low titers of IgG1 and GFP expression.

A vector containing the REP construct was digested using the restriction enzymes NheI and BstBI (BstBI is used at 65° C.). The REP fragment containing the expression construct was cut out, purified and used for further cloning steps.

### II.3. Cloning of Expression Vectors

The vector pGLEX41, an expression vector derived from pcDNA3.1 (+) (Invitrogen, Carlsbad, Calif.) was used for stable cell line production. It was used as initial backbone that had been modified to generate the second generations of vectors A and B with and without the GAPDH sequences. For all vectors the same promoter-intron combination (mCMV and a donor-acceptor fragment coding for the first intron (IgDA)) was used (Gorman et al., (1990) Proc Natl Acad Sci USA, 87: 5459-5463).

Cloning of Intermediate Vector pGLEX41-HM-MCS-ampiA:

The development of the new vector generation was started from pGLEX41. This vector was cut using the restriction enzymes NruI and BspHI in order to release the ampicillin resistance cassette. The backbone fragment was CIPed and purified by gel electrophoresis. The DNA fragment coding for a codon optimized (for expression in E. coli) version of the ampicillin resistance gene (including the bla promoter) has been ordered from GeneArt. The insert was cut out of the GeneArt cloning vector #1013237 using the restriction enzymes NruI and BspHI (the same enzymes as used for the backbone), purified and cloned into the backbone.

Minipreps were analyzed by restriction digest. The clone pGLEX41-HM-MCS-ampiA #2 had the expected restriction profile and the integration of the correct fragment was confirmed by sequencing.

Cloning of Intermediate Vector pGLEX41-MCS-R6K-ampiA

In order to exchange the pUC origin of replication of the vector pGLEX41-HM-MCS-ampiA #2 the vector was digested using PvuI and BspHI. The backbone fragment was CIPed and purified. The new insert fragment contains the R6K origin of replication and a modified SV40 poly(A) sequence as part of the expression cassette. Unnecessary bacterial or viral backbone sequences around the SV40 poly(A) had been eliminated (see Table 2 below). The insert fragment has been ordered from GeneArt; it was cut out of the GeneArt cloning vector #1013238 using the enzymes PvuI and BspHI (the same as used for the backbone), purified and cloned into the backbone fragment. Minipreps were prepared and were confirmed by sequence analysis. The clone pGLEX41-MCS-R6K-ampiA #1 had the correct sequence.

TABLE 2
Content of CpG in the different vectors

| | CpG content in expression vectors | | |
|---|---|---|---|
| | | Codon optimized | CpG reduced |
| | pGLEX41 | Vectors: “A” | Vectors “B” |
| Ampicillin resistance | 49 | 43 | 19 |
| Puromycin resistance | 93 | 36 | 1 |
| Geneticin resistance | 74 | 51 | 0 |
| Origin of replication | 45 | 9 | 9 |
| Sum: | 261 | 139 | 29 |

Cloning of Intermediate Vector pGLEX41-MCS-R6K-ampiB

The vector pGLEX41-MCS-R6K-ampiA # was opened using the restriction enzyme BspHI and CIPed in order to release the ampicillin resistance. The new insert fragment contains the ampicillin resistance codon optimized for expression in E. coli, but all the CpG sequences that could be eliminated by alternative codon usage had been replaced (see Table 2 above). This fragment was ordered at GeneArt. In order to release the insert fragment, the GeneArt cloning vector #1016138 was digested using BspHI. After purification of both insert and backbone fragments by gel electrophoresis, they were ligated and transformed into PIR1 bacteria. The minipreps were directly sent for sequencing. pGLEX41-R6K-MCS-ampiB #1 has the correct sequence and was used for further cloning steps.

Cloning of the Reporter Construct in pGLEX41-Derived Expression Vectors

In order to clone the reporter construct REP in the expression vectors pGLEX41-MCS-R6K-ampiA and pGLEX41-MCS-R6K-ampiB, the vectors were cut using the restriction enzymes NheI and ClaI. The expression vector pGLEX41-HM-MCS was opened using the restriction enzymes NheI and BstBI (at 65° C.). All vector backbones were treated with CIP after digestion and the backbones purified by gel electrophoresis. The backbones were ligated with the NheI/BstBI (BstBI is compatible with ClaI) fragment coding for the reporter construct REP. The ligation products were transformed into PIR1 or TOP 10 competent bacteria and plated on ampicillin LB-agar plates. Colonies were amplified and plasmids were isolated by minipreps. Positive clones could be identified by restriction digest of minipreps and subsequent sequence confirmation by Fasteris SA.

Addition of Flanking GAPDH Sequences in pGLEX41 Derived Expression Vectors

All restriction digest of this paragraph were performed in a 80 μl final volume and incubated over night at 37° C.

5′GAPDH sequence (SEQ ID NO: 7) was excised from pCR-blunt-5′GAPDH using the restriction enzyme NruI and ligated in the expression vectors pGLEX41-R6K-ampiA-[REP] and pGLEX41-R6K-ampiB-[REP] which were linearized using NruI and treated with CIP in order to avoid re-circularization. After amplification of PIR1 colonies (obtained by transformation of ligation products) minipreps were analyzed by restriction digest. Clones pGLEX41-R6K-ampiA-5′GAPDH-[REP] #2 and pGLEX41-R6K-ampiB-5′GAPDH-[REP] #1 showed bands of the expected size in the restriction analysis, were subsequently confirmed by sequencing and used for further cloning steps. These new vectors were then opened with ScaI and treated with CIP. The 3′GAPDH fragment (SEQ ID NO: 8) was excised from pCR-Blunt-3′GAPDH using the same enzyme and ligated into the two backbones in order to generate pGLEX41-R6K-ampiA-GAPDH-[REP] and pGLEX41-R6K-ampiB-GAPDH-[REP] expression vectors.

The control digest of clones pGLEX41-R6K-ampiA-GAPDH-[REP] #2 and pGLEX41-R6K-ampiB-GAPDH-[REP] #8 showed bands of the expected size in the restriction analysis. The insertion of the 3′GAPDH fragment in the correct orientation was subsequently confirmed by sequencing (Fasteris).

### II.4. Cloning of Resistance Vectors

Starting point for the cloning of the resistance vectors was the vector pGLEX-MCS-R6K-ampiA #1. As for expression of resistance genes a weak promoter is sufficient, the mCMV promoter was replaced by the SV40 promoter. The genes coding for the resistance genes were ordered from GeneArt SA (Regensburg. Germany) and either optimized for expression in Chinese hamster (puromycin: puroA and neomycin: neoA) or reduced in CpG content by selective codon usage (puromycin: puroB and neomycin: neoB).

Cloning of pGLEX-R6K-AmpiA-PuroA/PuroB:

In order to clone the puromycin resistance in the expression cassette, the vector pGLEX41-MCS-R6K-ampiA #1 was opened using the restriction enzymes NruI and XbaI followed by treatment with CIP. The insert fragment was ordered from GeneArt and was provided as insert in GeneArt cloning vector #1013239. It contains the SV40 promoter and the codon optimized gene for the puromycin resistance (for codon usage of CHO cells). The insert was cut out of the GeneArt cloning vector using the enzymes NruI and XbaI (the same as used for the backbone), purified and cloned into the backbone fragment. Minipreps were prepared and analyzed by restriction digest. The clone pGLEX-MCS-R6K-ampiA-puroA #1 showed the correct profile and could be confirmed by sequencing.

This vector was used for the cloning of the vector pGLEX-MCS-R6K-ampiA-puroB by exchange of the coding region for the puromycin resistance gene, while leaving the SV40 promoter. The new insert fragment contains a codon-optimized version of the puromycin gene, where all the CpG sequences that could be eliminated due to alternative codon usage had been replaced. The fragment has been ordered by GeneArt and was delivered in the cloning vector #1016139. In order to release the insert fragment, the GeneArt vector was digested using the restriction enzymes XbaI and NotI. The insert fragment was purified by gel electrophoresis and cloned into the backbone of pGLEX-MCS-R6K-ampiA-puroA, after release of the puromycin open reading frame by restriction digest using XbaI and NotI, followed by CIP treatment. The resulting vector pGLEX-MCS-R6K-ampiA-puroB #1 was confirmed directly by sequence analysis.

Cloning of the Vectors pGLEX-R6K-ampiA-NeoA and pGLEX-R6K-ampiA-NeoB

In order to clone the neomycin resistance in the expression cassette, the vector pGLEX-R6K-puroA #1 was opened using the restriction enzymes XbaI and NotI, followed by treatment with CIP. The insert fragments were ordered from GeneArt and were provided as inserts in GeneArt cloning vectors #1013242 (neoA) and #1026894 (neoB). They contain the codon optimized gene for the neomycin resistance for codon usage of CHO cells and the CpG reduced version of the neomycin resistance, respectively. The inserts were cut out of the GeneArt cloning vectors using the enzymes XbaI and NotI (the same as used for the backbone), purified and cloned into the backbone fragment. Minipreps were prepared and the clones were confirmed by sequencing.

Cloning of Vectors pGLEX-R6K-ampiB-NeoB and pGLEX41-R6K-ampiB-puroB:

The vector pGLEX41-R6K-puroB #1 was opened using the restriction enzyme BspHI and subsequently CIPed. The insert fragment contains the ampicillin resistance gene that was codon optimized for expression in E. coli, while all CpG sequences that could be eliminated due to alternative codon usage had been replaced. This fragment has been ordered at GeneArt and arrived in the cloning vector #1016138. In order to release the insert fragment the GeneArt cloning vector was digested using BspHI. After purification of both insert and backbone fragment by gel electrophoresis, they were ligated and transformed into PIR1 bacteria. The minipreps were directly sent for sequencing and could be confirmed (pGLEX41-ampiB-R6K-puroB #1).

The cloning leading to vector pGLEX-R6K-neoB-ampiB was done by opening pGLEX-R6K-neoB-ampiA using the restriction enzymes BspHI in order to create the backbone fragment. Digestion of pGLEX-R6K-ampiB-hygroB using the same restriction enzyme combination yielded the insert fragment coding for ampiB. The ampiB insert was cloned into the pGLEX-R6K-neoB-ampiA backbone.

II.5 Addition of Sequences Upstream and Downstream of the Human GAPDH Gene into Resistance Vectors

The vector pCR-blunt-5′GAPDH was digested with NruI in order to obtain the 5′GAPDH insert (3164 bps). The vectors coding for resistance genes were digested with NruI, subsequently treated with CIP (Calf intestinal phosphatase, NEB, Ipswich, Mass.) in order to prepare the backbone fragments. The 4 different backbone fragments (pGLEX-R6K-neoA-ampiA, pGLEX-R6K-neoB-ampiB, pGLEX-R6K-puroA-ampiA and pGLEX-puroB-ampiB) were ligated with the 3164 bps 5′GAPDH insert and transformed into PIR1 competent bacteria. Restriction digest of minipreps using ApalI allowed the identification of clones pGLEX-R6K-neoB-ampiB-5′GAPDH #5, pGLEX-R6K-neoA-ampiA-5′GAPDH #6, pGLEX-R6K-puroA-ampiA-5′GAPDH #16 and pGLEX-puroB-ampiB-5′GAPDH #5.

These intermediate vectors were then cut with the restriction enzyme ScaI and treated with CIP in order to prepare the backbones for ligation. The vector carrying the second insert fragment, pCR-Blunt-3′GAPDH, was cut using ScaI in order to release the insert fragment (3224 bps) the GAPDH downstream flanking region. The four different backbone molecules were ligated with the purified 3224 bps insert fragment and transformed into PIR1 competent cells. Minipreps were analyzed by restriction digest. Clones showing restriction fragments of the expected size were pGLEX-R6K-neoB-ampiB-GAPDH #8, pGLEX-R6K-neoA-ampiA-GAPDH #1, pGLEX-R6K-puroA-ampiA-GAPDH #1 and pGLEX-puroB-ampiB-GAPDH #4. The clones were subsequently confirmed by sequencing analysis (Fasteris, Geneva. Switzerland).

### II.1.5. Midipreparations of Plasmids Cloned for Transfection

In order to have sufficient quantities of plasmids, midipreps were prepared using the Macherey Nagel kit (NucleoBond Xtra Midi: Macherey Nagel, Oensingen, Switzerland). After confirmation by restriction digest and sequencing, the plasmids were linearized and used for transfection in CHO-S cells. Table 3 summarizes the concentrations of plasmid DNA batches obtained in midipreparations, linearized DNA preps that had been prepared for transfection, the enzymes used for linearization and the sequence files from Fasteris SA confirming the identity and the sequence information of the respective plasmid. All midipreps were confirmed by sequencing before being used for transfections.

TABLE 3
Summary of plasmids cloned. Concentration of DNA midipreparation
and linearized midipreparation (with the corresponding enzyme).
The GSC number codes for the respective plasmid and allows
to identify relevant sequencing files.

| | Conc. of | Enzyme | Conc. of | |
|---|---|---|---|---|
| | Midi- | for | linearized | Glenmark |
| | preparation | linear- | plasmids | plasmid |
| Plasmids | (μg/ml) | ization | (μg/ml) | code |
| pGLEX41-R6K-AmpiA- | 1538 | EcoRV | 1019 | GSC 2774 |
| [REP]-GAPDH | | | | |
| pGLEX41-R6K-AmpiB- | 1243 | EcoRV | 1233 | GSC 2775 |
| [REP]-GAPDH | | | | |
| pGLEX-R6K-AmpiA- | 890 | AseI | 766 | GSC 2776 |
| neoA-GAPDH | | | | |
| pGLEX-R6K-AmpiB- | 594 | AseI | 979 | GSC 2777 |
| neoB-GAPDH | | | | |
| pGLEX-R6K- AmpiA- | 917 | AseI | 859 | GSC 2778 |
| puroA- GAPDH | | | | |
| pGLEX-AmpiB-puroB- | 869 | AseI | 1049 | GSC 2779 |
| GAPDH | | | | |
| pGLEX41-[REP] | 2119 | BspHI | 868 | GSC 2239 |
| pGLEX41-R6K-AmpiA- | 865 | BspHI | 779 | GSC 2240 |
| [REP] | | | | |
| pGLEX41-R6K-AmpiB- | 1751 | BspHI | 806 | GSC 2249 |
| [REP] | | | | |
| pGLEX-R6K-AmpiA- | 890 | BspHI | 764 | GSC 2214 |
| neoA | | | | |
| pGLEX-R6K-AmpiB- | 767 | BspHI | 654 | GSC 2244 |
| neoB | | | | |
| pGLEX-R6K-AmpiA- | 708 | BspHI | 659 | GSC 2220 |
| puroA | | | | |
| pGLEX-R6K-AmpiB- | 574 | BspHI | 746 | GSC 2213 |
| puroB | | | | |

### Example 2: Transfection of Cells with Expression Vectors

### 1. Materials and Methods

### CHO-S Cells and HEK293 Cells

Mammalian cells are the preferred host to express proteins because they are capable of correct folding, assembly and post-transcriptional modification of recombinant proteins. The CHO cell line was used because they are well characterized and do not serve as a host for most human pathogenic viruses, making them a relatively safe host for stable therapeutic protein production. Chinese Hamster Ovary cells (CHO-S, Invitrogen, Carlsbad, Calif., USA) were cultured in suspension in PowerCHO-2 CD medium (Lonza, Verviers, Belgium), supplemented with 4 mM L-glutamine (Applichem, Germany) and incubated in a shaking incubator (200 rpm with a circular stroke of 2.5 cm) at 37° C., 5% CO₂ and 80% humidity. HEK293 cells are used because they are easy to transfect and allow rapid production of recombinant proteins up to lower gram amounts. The cells used are HEK293-EBNA cells (ATCC, Manassas, Va.) and are routinely cultured in suspension in Ex-cell 293 medium (Sigma-Aldrich, St. Louis, Mich.).

Subcultures of CHO-S and HEK293 EBNA cells were routinely carried out every 3-4 days using a seeding density of 0.5×10⁶ viable cells/ml in fresh medium. The cells were cultivated using 10 ml of medium in 50 ml bioreactor tubes (Tubespin Bioreactor 50; TPP, Trasadingen. Switzerland) containing a permeable filter allowing gas exchange. The cell viability and concentration were determined with the Countess automated cell counter (Invitrogen, Carlsbad, Calif., USA) using the trypan blue cell exclusion method. Cell concentration was confirmed by determination of the packed cell volume (PCV) method using PCV tubes (TPP, Trasadingen, Switzerland) for CHO-S cells.

### Packed Cell Volume (PCV)

The PCV method is based on the centrifugation of a specific volume of culture liquid in a mini-PCV tube (PCV Packed Cell Volume Tube: TPP, Trasadingen, Switzerland) for 1 min at 5000 rpm. During centrifugation, the cells are pelleted in the graduated capillary at the base of the tube. The percentage of packed cell volume is then determined by assessing the volume of the pellet in relationship to the amount of cell culture fluid centrifuged. For example, 1% PCV indicated that 10 μl of cell pellet was present in 1 ml of culture fluid.

For routine cell counting of cells, 200 μl of each sample was pipetted in a PCV tube and the volume of the corresponding pellet (in μl) was read with a ruler (“easy read” measuring device; TPP, Trasadingen, Switzerland). This volume was multiplied by 5 to have the value for 1 ml and then it was multiplied using a cell specific correlation factor to obtain an estimation of the concentration of viable cells (in millions of cells/ml).

### “Automatized” Cell Counting

Cell concentration and viability was determined with the Countess® Automated Cell Counter (Invitrogen. Carlsbad, Calif., USA) in mixing the sample with the same amount of trypan blue. The solution is then pipetted into the Countess® chamber slide before being read by the instrument. This instrument allows an automatic read-out of the Neubauer chamber which, after calibration, determines cell viability and the concentration of dead and living cells.

### Flow Cytometry Analysis

Flow Cytometry is a technique for the analysis of multiple parameters of individual cells. This technique allows the quantitative and qualitative analysis of cells that are phenotypically different from each other, for instance dead from viable cells (according to the size and the granularity of cells). It also allows the quantification of cells which express a protein of interest, such as GFP. Cells were collected from the culture by sterile pipetting 300 μl of samples and were analyzed with a Fluorescence-Associated Cell Sorting (FACS) Calibur flow cytometer (Becton, Dickinson and Company, Franklin Lakes, N.J., USA) equipped with an air-cooled argon laser emitting at 488 nm. The analyses were made with the CellQuest software. GFP emission was detected with the FL-1, using a 530/30-nm band pass filter.

In the first gate, cell debris as well as dead cells were excluded from the analysis in a SSC/FSC dotplot on linear scale. Then, the GFP fluorescence of living cells was displayed in a histogram on logarithmic scale. The median value of the fluorescence distribution was used to assess the GFP expression level of the analyzed cell populations.

### IgG Quantification Method: OCTET QK

The Octet QK system (ForteBio, Menlo Park, Calif., USA) performs label-free quantitation of antibodies, proteins, peptides, DNA and other biomolecules and provides kinetic characterization of biomolecular binding interactions. A correlation between the binding rate (nm) and the accumulated IgG1 concentration (μg/ml) of the sample allows quantification of the IgG titer with a calibration curve.

Cell samples were centrifuged 5 min at 300 g. The supernatant was then diluted (⅕ for IgG1 antibody) with the Octet Buffer in a 96 well plate before being analyzed with the Octet using Protein A biosensors (Protein A DIP and READ™ Biosensor, Forte Bio, USA) to obtain the antibody concentration per well.

### Transient Transfection Using JetPEI

Transient and stable transfection of CHO-S and HEK293 EBNA cells was performed using polyethyleneimine (PEI; JetPEI, Polyplus-transfection, Illkirch, France). PEI is a cationic polymer which can complex with negatively charged molecules such as DNA. The positive charged DNA-PEI complex binds to the negatively charged cell surface and is internalized by endocytosis. It reaches the lysosome compartment from where it is released by lysis to the nucleus. The high transfection efficiency with DNA-PEI complexes is due to the ability of PEI to protect DNA from lysosomal degradation. The cells were transfected according to the manual provided by the manufacturer.

All plasmids were linearized before stable transfection (100 μg of DNA resuspended in 100 μl Tris-EDTA, pH 7.5). For transient transfection circular plasmids were directly used from midipreparation DNA. In this study, transient transfections were kept in 50 ml bioreactor tubes and no antibiotics were added.

Stable CHO-S clones expressing IgG1 and GFP were obtained by co-transfecting one expression vector and two resistance vectors (coding for puromycin or neomycin resistance, respectively).

### Selection of Stable Pools and Minipools

Transfection efficiency was determined 24 h after transfection by Flow Cytometry (BD FACS Calibur cytometer, #1293) by analysing the intracellular GFP expression. If the percentage of GPF positive cells was higher than 20%, the transfected cells were diluted with selective medium and distributed into 96 well plates (for limiting dilution to generate isolated stable minipools) or in T-Flasks (to generate stable pools). The selective medium used was PowerCHO-2, 4 mM glutamine, supplemented with different concentrations of geneticin and puromycin.

Seven days after transfection, the selection stringency was renewed by adding selection medium to the cells. As soon as colonies in 96 well plates were confluent, the plates were read using a fluorescence reader.

The pools in T-Flasks were expanded to tubespin scale using antibiotic-free PowerCHO-2, 4 mM L-glutamine. Their viability and concentration were evaluated with the Countess automated cell counter (Invitrogen, Carlsbad. Calif., USA). As soon as the cell density allowed it, a seed train was started for every pool by seeding cells at a density of 0.5×10⁶ cells/ml in 10 ml medium in 50 ml bioreactor tubes (incubated in a shaker (200 rpm) at 5% CO₂, 37° C. and 8⁰% humidity). Each seed train was passaged twice a week by seeding the cells at 0.5×10⁶ cells-ml in growth medium (cell concentration was determined by PCV analysis). The seed train was used for the inoculum of all productions runs (batches).

For the next 4-5 weeks productions runs were seeded once a week in duplicates. The pool stability was evaluated by FACS and IgG expression as described above for clonal populations.

### Production Runs (Batch Fermentation)

The batch runs of cell pools were seeded at a concentration of 0.5×10⁶ cells/ml using the seed train for inoculation and cells were then cultured for 7 days in Feed media. On day 4 and 8, 200 μl of cells were centrifuged for 5 min at 300 g and the supernatant was analyzed for accumulated IgG using the Octet. In addition, the GFP expression of each batch was analyzed by FACS.

### 2. Results

### 2.1 Expression in Transient in CHO Cells:

The vectors compared in this study differ mainly in their backbone. The entire expression cassette (Promoter, first intron, expression construct, poly (A)) is exactly the same for all vectors. The vectors are derived from the vector pGLEX41 as described in Example 1. In one vector, the ampicillin resistance gene was codon optimized for expression in E. coli and the bacterial backbone was reduced to a minimum: pGLEX41-R6K-AmpiA-[REP] (in short A). In a second vector, the ampicillin resistance gene was codon optimized for expression in E. coli, but all CpG sequences were avoided, by using alternate codons (when possible): This vector is called pGLEX4 I-R6K-AmpiB-[REP] (in short B). The third modification included the use of the GAPDH flanking sequences that were cloned upstream and downstream of the expression cassette of the vectors A and B giving the vectors pGLEX41-R6K-AmpiA-[REP]-GAPDH (in short GAPDH_A) and pGLEX41-R6K-AmpiB-[REP]-GAPDH (in short GAPDH_B).

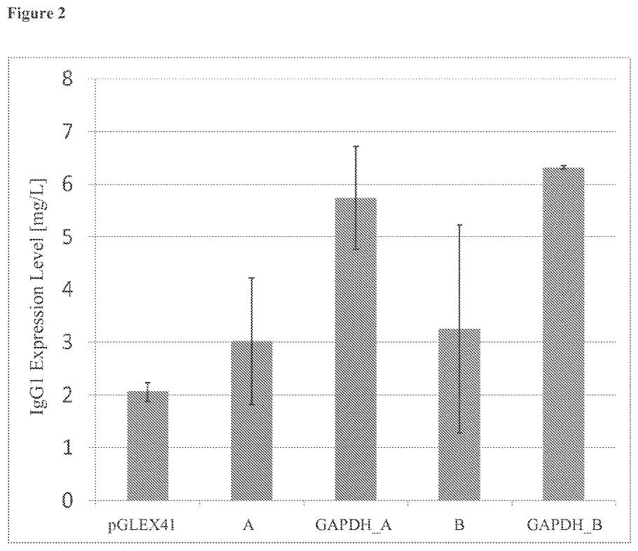

Transient transfections of CHO-S cells (Invitrogen) were done in order to compare the expression level of the reporter proteins expressed in the context of the different plasmid backbones. The transfections (in duplicate) were performed in 50 ml bioreactor tubes (TPP, Trasadingen, Switzerland) using 10 ml of final medium volume and analyzed on day 5 after transfection by Octet (FIG. 2).

All vectors (A and B) with corrected backbone show a slightly higher expression level than the control vectors pGLEX41. There is only a minor difference between the vectors A and B. This is expected, because the only difference in the backbone is the ampicillin resistance which should not have an impact on transient expression.

The most striking observation is the positive effect of the GAPDH sequences on expression. A 2-fold higher expression level is obtained with the plasmid harbouring the GAPDH flanking sequences compared to the ones without the GAPDH sequences. This is true for both A and B constructs. Compared to the pGLEX41 vector, a 3-fold higher expression can be observed. This is even more surprising if the size of the plasmids is taken into account. The vector A (7048 bps) is almost half the size compared to the vector GAPDH-A (13436 bps). Therefore, assumed that the amount of delivered DNA during the process of transient transfection is the same for all plasmids, only half the molar amount of GAPDH-A is delivered to the nucleus.

### 2.2 Expression in Transient in HEK293 Cells

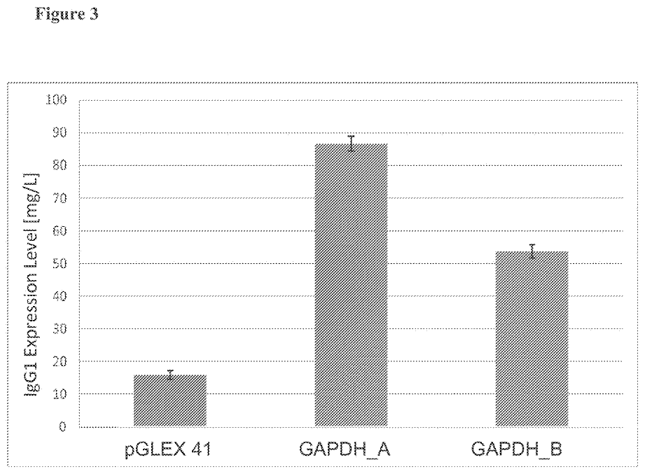

Transient transfections of HEK293 EBNA cells were done in order to compare the expression level of the reporter proteins expressed in the context of the different plasmid backbones. The transfections (in duplicate) were performed in 50 ml bioreactor tubes (TPP, Trasadingen, Switzerland) using 10 ml of final medium volume and were analyzed on day 10 after transfection by Octet (FIG. 3).

The results shown in FIG. 3 show a significant increase in expression that can be obtained using the GAPDH flanking regions in HEK293 EBNA cells. The GAPDH-B vector is showing a threefold increase in expression, whereas the GAPDH-A vector shows an even higher increase in expression of 5-fold. These vectors do not contain the oriP element and might therefore have a potential for even higher titers.

### 2.3 Expression in Stable CHO Cell Lines

### Establishment of Stable Transfected Cells

Stable populations were generated by co-transfecting an expression vector and vectors coding for resistance genes, followed by selection pressure mediated by antibiotics. The selection pressure was removed 14 days after transfection. These steps allowed the generation of stable minipools and stable pools which were cultured in regular intervals in production runs in order to compare the expression levels of the reporter proteins (IgG1 antibody and GFP) of the different constructs and the stability of expression.

Reporter Protein Expression Study on Production Runs Performed with Cell Pools

Pools were generated by stable transfection. During the selection procedure (the first 14 days after transfection) the pools were analyzed by FACS. An increase of the GFP positive cell fraction together with the viability of the culture could be observed over the time. The selection pressure mediated by the antibiotics was removed from the pools after 14 days. Using this approach no cell pools transfected with the “B” plasmids could be obtained. The expression level of the generated pools was assayed as soon as the cells could be cultured in 50 ml bioreactor tubes. Batches were done in duplicates. The cells were analyzed by FACS for GFP expression and the accumulation of IgG in the supernatant was assayed by Octet after 8 days of expression.

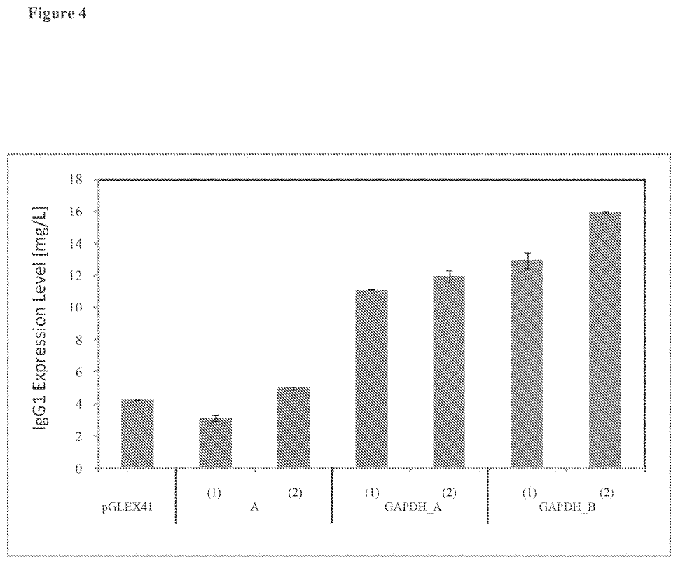

A proportional relationship could be observed between the IgG titers and the GFP expression of the pools. Therefore, only the IgG data are shown in FIG. 4. All pools transfected with vectors containing GAPDH sequence show higher expression compared to the vector pGLEX41 or with the same vector without GAPDH sequence (factor of 2.8 between A and A-GAPDH. No conclusion could be drawn between B and B-GAPDH as no B pools survived). Transfections performed with A-GAPDH and B-GADPH induced a higher expression of IgG (2.7 and 3.5 folds more respectively) than pGLEX41 transfection (for batch-2). Therefore in pools, the GAPDH flanking sequences seem to be favourable for the production of proteins. Finally, transfections performed with B-GADPH vectors induced a higher expression of IgG than the transfection performed with A-GAPDH (factor of 1.25). Therefore, the CpG reduction in resistance genes seems to be favourable for the stable production of proteins, too.

### Expression Level Study on Clonal Populations

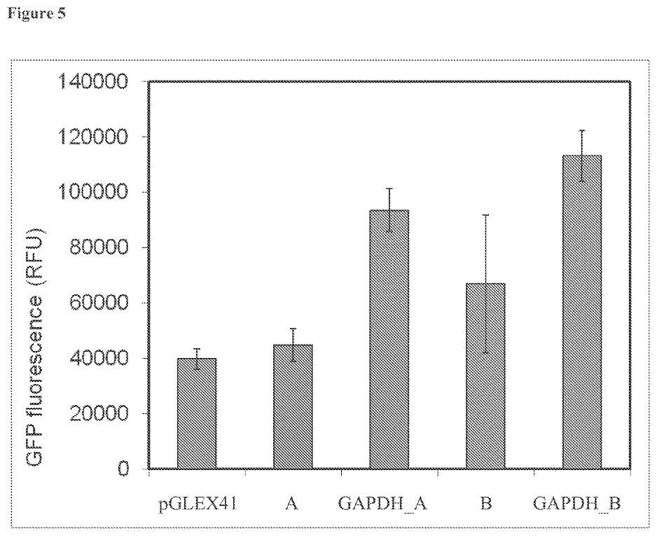

Cells were transfected and distributed in 96 well plates in selective medium in order to obtain clonal or oligoclonal populations. After 7 days the selection pressure was refreshed by addition of selective medium to the cells. The expression of GFP was assessed 14 days after transfection by using an ELISA-plate reader. The results are shown in FIG. 5.

Confirming the results obtained in cellular pools, cells transfected with vectors containing GAPDH flanking sequences expressed significantly more GFP than the same backbone without GAPDH up- and downstream sequences (factors from 1.7 to 2 fold) or the other vectors used as control (pGLEX41: 2.5 fold) (FIG. 5). In addition, populations with vectors containing resistance sequences which had been CpG reduced (B) induced a higher expression than the corresponding vectors which had only been codon optimized (A) (1.5 fold between A and B; 1.2 fold between B and B-GAPDH).

From the expression study several conclusions could be drawn. First, the GAPDH up- and downstream sequence allows higher expression than the standard vector that was used as a benchmark (pGLEX41). Also a lower expression level is obtained when cells are transfected with the same vector backbone without the GAPDH sequences confirming that the beneficial effect on the expression is related to the inserted GAPDH flanking sequences. In addition, the reduction of CpG number in the expression and selection plasmids seems to be slightly favourable for expression, too.

### Example 3: Transient Expression Level of CHO-S GMP Cells Transfected with New Designed Vectors

It has been described in the literature that the 5′ region of the GAPDH promoter harbours a potential insulin as well as a phorbol ester response element (Alexander-Bridges et al., (1992) Advan Enzyme Regul, 32: 149-159). The phorbol ester response element (−1040-1010 bps) is situated upstream of what is usually referred to as the GAPDH promoter (−488-+20). In a deletion study performed in stable H35 Hepatoma cell lines, the authors were not able to demonstrate a significant effect of the deletion of basepairs −1200 to −488 (relative to the transcription starting point). Therefore the phorbol ester response element might not be functionally linked to the expression driven from the GAPDH promoter. Nevertheless a transient transfection experiment was performed in order to evaluate the contribution of insulin and PMA (phorbol-12-myristate-13-acetate, the most common phorbol ester) in the increase in transient and stable expression that was observed using the plasmids containing the GAPDH flanking elements.

In order to obtain insulin free growth medium. PowerCHO2 was prepared from powder medium and no insulin was added. PMA was purchased from Sigma (St. Louis, Mo.), and was dosed at a final concentration of 1.6 μM (corresponding to the concentration used by Alexander-Bridges on H35 Hepatoma cell lines) in PowerCHO2 (+/− Insulin).

Transfections were performed in 50 ml bioreactor tubes (Tubespins, TPP, Trasadingen, Switzerland) as described previously. In order to avoid the presence of insulin provided by OptiMEM (Life technologies, Carlsbad, Calif.), the transfection medium was changed to RPMI1640 (PAA, Pasching, Austria) supplemented with 4 mM Gln and 25 mM HEPES. After transfection, the cells were distributed in 12 well plates and 1 ml of the four different media was added (PowerCHO2, 4 mM Gln, +/−insulin; PowerCHO2, 4 mM Gln, 1.6 μM PMA, +/−insulin). Again, the reporter construct expressing IgG1 and GFP using two IRES was used (described in example 2). This vector allowed verification of the transfection efficiency. The percentage and the viability of transfected cells were found similar in all four different media preparations.

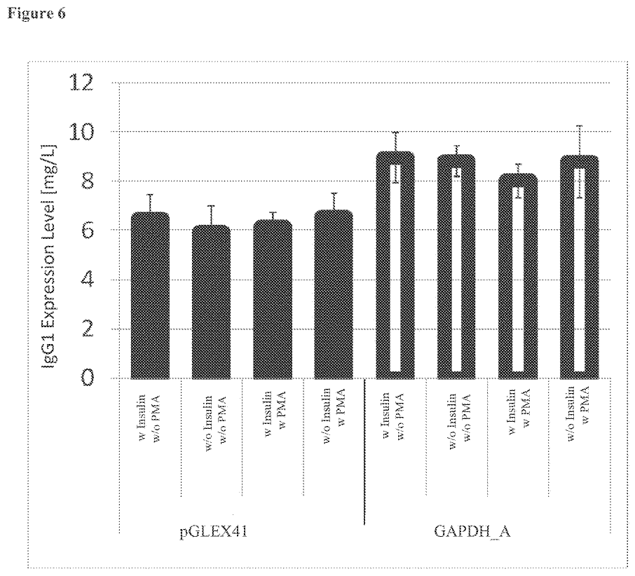

As shown in FIG. 6, no significant effect of insulin depletion and/or PMA addition could be observed during this experiment. Similar titers were obtained in all media used for expression. This suggests that the potential phorbol ester and the insulin response elements present in the upstream flanking sequence of the GAPDH gene do not affect transient transgene expression.

### Example 4: Fragmentation Analysis of DNA Flanking the GAPDH Expression Cassette Upstream of the Promoter and Downstream of the PolyA Site in Order to Study the Effect on Reporter Gene Expression

The human GAPDH locus is located on chromosome 12 of the human genome. GAPDH is described to be constitutively active in all cells of mammalian origin, as the enzyme is a key player in the metabolism of glucose. Upstream of the promoter, the GAPDH gene is flanked by NCAPD2, a gene that stretches over more than 30000 bps. Downstream of the polyadenylation site, the GAPDH gene is flanked by IFF01 (see FIG. 7 for details).

Not only GAPDH and the promoter, but also the flanking regions are well conserved between different species (see Table 4).

TABLE 4
Stretches of high homologies between human, rat and mouse GAPDH flanking regions.
Analysis was done using clone manager 9 (ScieED, Cary, NC, USA). The numbering is
relative to the first base of the upstream or the downstream flanking element,
respectively (Sequence ID NO: 7 and Sequence ID NO: 8, respectively). Sequences
used for alignment were for mouse bases 532-3731 (upstream) and 8164-11364
(downstream) of Sequence ID No 18 and for rat bases 719-3918 (upstream) and
8495-11058 (downstream) of Sequence ID No 19.

| Upstream region | | | | | | | |
|---|---|---|---|---|---|---|---|
| | | Sequences of | | Downstream | | | |
| Sequences of | | homology | | Sequences of | | Sequences of | |
| homology [rat] | | [mouse] | | homology [rat] | | homology [mouse] | |
| >80% | >90% | >80% | >90% | >80% | >90% | >80% | >90% |
| 161-249 | 279-331 | 15-69 | 278-329 | 1608-1764 | 1706-1764 | 1614-1671 | 1904-2061 |
| 256-338 | 554-623 | 159-249 | 546-626 | 1894-2067 | 1912-2061 | 1888-2072 | 2927-3071 |
| 515-659 | | 273-342 | | | | 2918-3082 | |
| 2296-2349 | | 515-647 | | | | | |
| 2381-2513 | | 1143-1223 | | | | | |
| 2736-2818 | | 1957-2009 | | | | | |
| | | 2029-2080 | | | | | |
| | | 2375-2485 | | | | | |
| | | 2730-2821 | | | | | |

A comparison of the DNA homology between rodent and human shows a minimum of DNA conservation of 38%. The presence of a conserved stretch of DNA outside of a promoter region or a region coding for a gene indicates that there might be a selection pressure on the cell to maintain the DNA sequence or to allow only certain/minor changes. In our specific case, the GAPDH flanking regions might be important for the cells because they maintain a high expression level of the GAPDH genes. Changes in the DNA sequence leading to decrease of expression would be selected against.

In order to evaluate the contribution of the upstream and the downstream GAPDH element to the observed increase in expression, constructs were made containing only the upstream GAPDH flanking region (SEQ ID NO: 7), fragments of the upstream GAPDH flanking region or the downstream GAPDH flanking region (SEQ ID NO: 8). The reporter IgG1 type antibody was expressed by an IRES construct (Light chain-IRES-heavy chain), therefore avoiding co-transfection of multiple plasmids. Details on the fragmentation of the GAPDH upstream fragment are shown in FIG. 8. The following fragments of the upstream GAPDH flanking region were used: Fragment 1 (SEQ ID NO: 9), fragment 2 (SEQ ID NO: 10), fragment 3 (SEQ ID NO: 11), fragment 4 (SEQ ID NO: 12), fragment 8 (SEQ ID NO: 13), fragment 9 (SEQ ID NO: 14), fragment 11 (SEQ ID NO: 15), fragment 17 (SEQ ID NO: 16).

The upstream GAPDH flanking region (SEQ ID NO: 7) used does contain 2 times 3 (in total 6) nucleotides of the NruI restriction site of which three are linked to the genomic DNA at its 5′ and three are linked to the genomic DNA its 3′ end. The downstream GAPDH flanking region (SEQ ID NO: 8) used does contain two times 3 (in total 6) nucleotides of the ScaI restriction site of which three are linked to the genomic DNA at its 5′ and three are linked to the genomic DNA its 3′ end. The upstream GAPDH flanking region and the downstream GAPDH flanking region without the nucleotides of the respective restriction site are shown in SEQ ID NO: 20 (upstream GAPDH flanking region without restriction sites) and SEQ ID NO: 21 (downstream GAPDH flanking region without restriction sites). The fragments of the upstream GAPDH flanking region used does each contain 3 nucleotides of the respective restriction site at its 5′ and/or its 3′ end linked to the genomic DNA (Fragment 1 contains 3 nucleotides of the NruI restriction site at its 5′end; Fragment 2 contains 3 nucleotides of the NruI restriction site at its 3′end; Fragment 3 contains 3 nucleotides of the NruI restriction site at its 5′end: Fragment 4 contains 3 nucleotides of the NruI restriction site at its 3′end; Fragment 8 contains 3 nucleotides of the NruI restriction site at its 3′end; Fragment 9 contains 3 nucleotides of the NruI restriction site at its 5′end and 3 nucleotides of the NruI restriction site at its 3′end; Fragment 11 contains 3 nucleotides of the NruI restriction site at its 3′end). Fragment 17 does not contain nucleotides of a restriction site. The fragments of the upstream GAPDH flanking region without the nucleotides of the respective restriction site are shown in SEQ ID NO: 22 (fragment 1 without restriction site), SEQ ID NO: 23 (fragment 2 without restriction site) SEQ ID NO: 24 (fragment 3 without restriction site), SEQ ID NO: 25 (fragment 4 without restriction site), SEQ ID NO: 26 (fragment 8 without restriction site), SEQ ID NO: 27 (fragment 9 without restriction sites), SEQ ID NO: 28 (fragment 11 without restriction site).

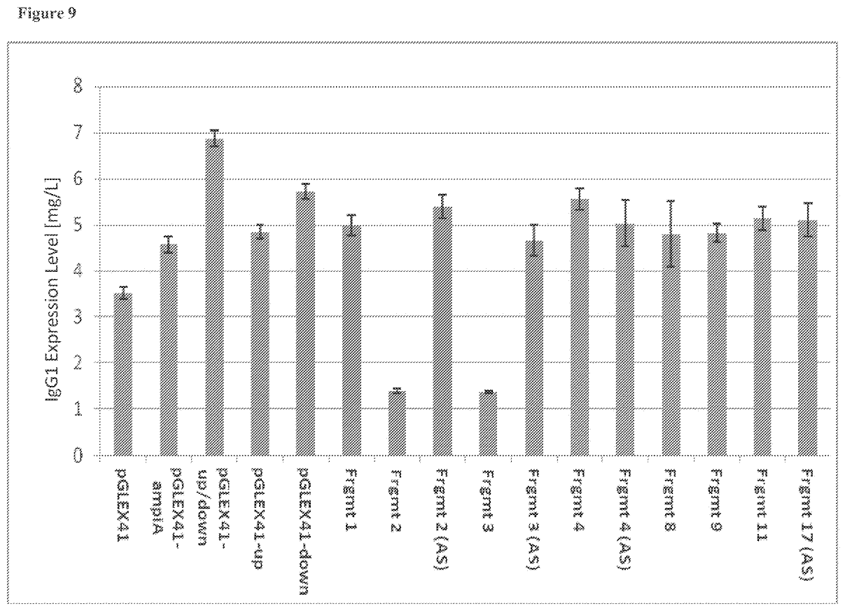

The effect of the upstream and the downstream GAPDH elements on expression was assessed on day 10 after transfection using the Octet (Fortebio, Menlo, Calif., USA) in order to quantify the amount of secreted IgG1 in the supernatant (see FIG. 9). pGLEX41, the original vector is giving lower expression results (80%) compared to the improved new vector design used in the pGLEX41-ampiA backbones. Compared to the original pGLEX41 backbone the new design includes codon optimization of the ampiA gene necessary for ampicillin resistance in E. coli, a different origin of replication (R6K instead of pUC origin of replication) and elimination of unnecessary linker (or spacer) sequences of bacterial origin. Both vectors have approximately the same size.

Surprisingly, pGLEX41-ampiA including the upstream (SEQ ID NO: 7) and downstream element (SEQ ID NO: 8), (named pGLEX41-up/down in FIG. 9 showing the expression results) is giving higher expression (factor 1.5) compared to the same vector without the upstream and downstream sequences. If one considers the difference in size (up/down fragments increase the size of the plasmid by approximately 6000 bps) and therefore the differences in delivered plasmid copies during transfection, the effect might even more important on a per plasmid basis.

The vector containing only the upstream fragment (up) is showing an expression level similar to the original expression construct pGLEX41-ampiA. The vector containing only the downstream fragment (down) is showing a significant increase (factor 1.2) in expression compared to the original expression construct pGLEX41-ampiA. A further increase in expression can be observed if both, the up- and the downstream fragment are present. This is confirmed by the fragmentation of the upstream fragments. Fragment 9 and the promoter proximal fragment 8 do not show any difference in expression compared to pGLEX41-ampiA. Fragment 1, 11 and 17 show an increase in expression. The highest increase was observed for fragment 4. It should be highlighted that the promoter proximal fragment 8 is not showing any effect. Therefore the increase in expression cannot be explained by previously published sequences (Alexander-Bridges et al., (1992) Advan Enzyme Regul, 32: 149-159), Graven et al., (1999) Biochimica et Biophysics Acta 147: 203-218).

Interestingly, fragments 2 and 3 lead to a significant decrease in expression. This is unexpected, especially in view of the fact that these fragments cloned in the opposite direction (antisense (AS) in FIG. 9) do not cause this effect. For the fragments 1, 8, 9, 11 and 17 no difference in expression was observed for fragments that were integrated in sense or antisense orientation (data not shown). Fragment 11, although a part of fragment 2, does not show this effect. Therefore the sequence element that seems to be detrimental to expression should be at least partially on the BstBI-BstBI fragment that was deleted in fragment 2 in order to obtain fragment 11.

In addition, the hypothesis that a negative element is located (at least partially) on the BstBI-BstBI fragment is supported by the increase in expression observed between fragment 3 (which includes the BstBI-BstBI fragment) and fragment 1.

While it seems easy to localize the fragment having a negative effect (BstBI-BstBI), from this study it is less obvious how this negative effect observed for fragment 2 and 3 is compensated by sequence elements present in the complete upstream fragment. It could be that this negative effect is balanced out by the small positive effect that was observed by fragment 1 and fragment 4 (but the increase in expression for fragment 1 is less than for fragment 4). Nevertheless the positive effect for fragment 4 (factor 1.25) observed seems less important compared to the negative effect (factor 0.4). Furthermore fragment 9, which is the entire upstream region without the BstBI-BstBI fragment does not show increased expression compared to the entire GAPDH upstream flanking region (nevertheless, fragment 9 includes the EcoRV-BstBI fragment which is part of fragment 2 and 3 and might have a negative effect on expression).

It can only be speculated about the mechanism behind the observed effects. The orientation dependency of the negative effect on expression observed with fragments 2 and 3 excludes the expression of non-identified open reading frames (for example expression of an ncRNA), because there are no surrounding promoters that could trigger the expression of only one orientation. The fact that the expression is reduced below the basal level shows not only the absence of a positive effect (for example an enhancer activity), but rather the presence of an orientation dependent negative effect.

In summary, a surprising increase of expression in transient in CHO cells is observed if both flanking regions, the upstream and the downstream region, are present in the expression plasmid. Although fragment 4 seems to have a significant positive effect on expression, no single fragment could be identified that is responsible for the entire increase of expression that was observed. The increase of expression of the expression vector pGLEX41-ampiA (up/down) seems to be the summary effect of both, up- and downstream flanking region.

### Example 5: Cloning of the Non-Translated Genomic DNA Sequence Upstream of the Chinese Hamster GAPDH Gene and the Chinese Hamster Promoter

1.1 Cloning of the Non-Translated Genomic DNA Sequence Upstream of the Chinese Hamster GAPDH Gene into an Expression Vector

The non-translated genomic DNA sequence upstream of the Chinese hamster GAPDH gene was amplified from genomic DNA of CHO-S(Life Technologies) cells by PCR. Genomic DNA was extracted as described in Example 1. Constructs were prepared using the mouse CMV promoter or the Chinese hamster GAPDH promoter for the expression of the reporter gene construct [REP] described in Example 1.

For cloning of the genomic DNA sequence upstream of the Chinese hamster GAPDH gene in combination with the mouse CMV promoter, primers GlnPr1896 and GlnPr1897 were used for amplification of the 3 kbs fragment (bps 672 to 3671 of SEQ ID No 29) using the PCR protocol described in Example 1 and leading to the amplicon with the SEQ ID No 30. The amplicon contains the genomic DNA sequence upstream of the Chinese hamster GAPDH gene and 5′ and 3′ restriction sites that were introduced by the primers.

For cloning of the genomic DNA sequence upstream of the Chinese hamster GAPDH gene in combination with the Chinese hamster GAPDH promoter, primers GlnPr1902 and GlnPr1905 were used in order to amplif, the 3508 bps fragment containing the genomic DNA sequence including the genomic DNA sequence upstream of the Chinese hamster GAPDH gene and the GAPDH promoter (bps 672 to 4179 of SEQ ID No 29) leading to the amplicon with the SEQ ID No 31. In a second PCR, GlnPr1901 and GlnPr1902 were used for amplification of the 508 bps fragment containing only the promoter region (bps 3672 to 4179 of SEQ ID No 29), leading to the SEQ ID No 32. The intron used in the vector “A” (described in Example 1) was amplified using primers GlnPr1903 and GlnPr1904.

A first fusion PCR was performed with primers GlnPr1904 and GlnPr1901 using the amplicon with SEQ ID NO: 32 and the amplicon with the intron sequence as templates. The amplicon contains the Chinese hamster GAPDH promoter, an intron and 5′ and 3′ restriction sites that were introduced by the primers. All primers are shown in Table 5.

A second fusion PCR was performed with primers GlnPr1905 and GInPr1904 using the amplicon with SEQ ID No. 31 and the amplicon with the intron sequence as templates. The amplicon contains the genomic DNA sequence upstream of the Chinese hamster GAPDH gene, the Chinese hamster GAPDH promoter, an intron and 5′ and 3′ restriction sites that were introduced by the primers.

After purification on a 1% agarose gel, the bands of interest were cut out and purified using the kit “NucleoSpin Gel and PCR Clean-up” (Macherey Nagel, Oensingen, Switzerland). The purified fragments were cloned into the plasmid pCR_Blunt using the Zero Blunt PCR cloning Kit (Invitrogen, Carlsbad, Calif. USA). Ligation products were transformed into competent E. coli TOP 10 (One Shot® TOP 10 Competent E. coli; Invitrogen, Carlsbad, Calif., USA) and analyzed by restriction analysis of minipreps. This led to the plasmids pCR_blunt[CHO-upstreamGAPDH], containing the genomic DNA sequence upstream of the Chinese hamster GAPDH gene, pCR_Blunt[CHO-upstreamGAPDH_GAPDHpromoter] containing the genomic DNA sequence upstream of the Chinese hamster GAPDH gene and the GAPDH promoter and intron from vector “A” and pCR_Blunt[CHO-GAPDHpromoter] containing the GAPDH promoter and the intron from vector “A”.

For evaluation of the amplicons on their effect on expression of a secreted gene, the vector “A” (described in Example 1) was used. As described previously, the expression cassette used in this vector contains a polycistronic gene coding for a secreted IgG1 and GFP (see Example 1). Transfected cells will therefore secrete the IgG1 monoclonal antibody and accumulate intracellular GFP in a dependent manner.

In order to release the 3 kb insert fragment containing the genomic DNA sequence upstream of the Chinese hamster GAPDH gene, the plasmid pCR_Blunt[CHO-upstreamGAPDH] was digested using the restriction enzyme NaeI. This insert was cloned in the backbone of “A”, digested using the restriction enzyme NruI and CIPed (CIP: NEB, Ipswich, Mass. USA). Backbone and insert were ligated together using T4 DNA ligase (T4 DNA ligase, NEB, Ipswich. Mass., USA) and subsequently transformed into competent E. coli PIR1. Clones were picked for miniprep preparation and subsequent restriction analysis. The resulting plasmid was called “A_GAPDH_UP”, confirmed by sequencing analysis and produced in midiprep scale using the NucleoBond Xtra Midi kit (Macherey Nagel. Oensingen. Switzerland).

For the cloning of expression constructs using the Chinese hamster GAPDH promoter, the insert fragments were released from plasmids pCR_Blunt[CHO-upstreamGAPDH_GAPDH promoter] and pCR_Blunt[CHO-GAPDHpromoter] by digestion using the restriction enzymes NheI and NruI. The resulting fragments were cloned in the backbone of vector “A”, opened using the same enzymes and CIPed. After ligation with T4 DNA ligase and transformation into competent E. coli PIR1, clones were picked for miniprep restriction analysis. The resulting plasmids were called “A_GAPDH_UP_Prom” (plasmid with non-translated genomic DNA sequence upstream of the Chinese hamster GAPDH and the promoter) and “A_PR” (plasmid with only the promoter) confirmed by sequencing analysis and produced in midiprep scale using the kit NucleoBond Xtra Midi (Macherey Nagel, Oensingen, Switzerland).

### 2. Assessment of the Effect of the Non-Translated Genomic DNA Sequence Upstream of the Chinese Hamster GAPDH Gene on the Expression of the Reporter Gene Construct

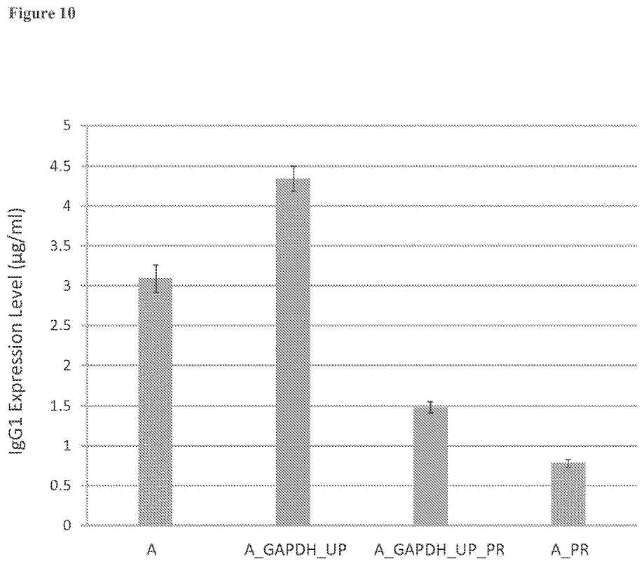

CHO-S cells were transfected in tubespins bioreactors using 10 ml of medium volume (as described in Example 2). The transfected cells were incubated in a shaking incubator with 200 rpm agitation at 37° C., 5% C02 and 80% humidity. The supernatants of the cells were analyzed for IgG1 expression using the Octet QK system with Protein A biosensors, (FortéBio, Menlo Park, Calif., USA). The results are shown in FIG. 10.

The expression level of the plasmid containing the GAPDH promoter (“A_PR”) compared to the mouse CMV promoter (A) is reduced by 50%, indicating that the Chinese hamster GAPDH promoter is not as strong as the viral promoter. The plasmid containing the non-translated genomic DNA sequence upstream of the Chinese hamster GAPDH gene in combination with the Chinese hamster GAPDH promoter (“A_GAPDH_UP_Prom”) shows a two fold increase in expression compared to the construct having only the GAPDH promoter (“A_PR”). The plasmid containing the non-translated genomic DNA sequence upstream of the Chinese hamster GAPDH gene and the mouse CMV promoter “(A_GAPDH_UP”) shows the highest expression and an increase of more than 40% over the plasmid containing only the mouse CMV promoter (“A”). This confirms that the non-translated genomic DNA sequence upstream of the Chinese hamster GAPDH gene has an enhancer effect on the expression of the reporter protein.

TABLE 5
Primers used for cloning in Example 5

| | SEQ | | Orien- | Restrn. |
|---|---|---|---|---|
| Primer | ID No | Sequence | tation | site |
| GlnPr | SEQ ID | TACGGCCGGCTTCACTGTA | forward | NaeI |
| 1896 | No 33 | CAGTGGCACAT | | |
| GlnPr | SEQ ID | TCAGGCCGGCCGTGGTTCT | reverse | NaeI |
| 1897 | No 34 | TCGGTAGTGAC | | |
| GlnPr | SEQ ID | TACTCGCGAAGAAGATCCT | forward | NruI |
| 1901 | No 35 | CAACTTTTCCACAGCC | | |
| GlnPr | SEQ ID | GTTCACTAAACGAGCTCTG | reverse | / |
| 1902 | No 36 | CTATTTATAGGAACTGGGT | | |
| | | G | | |
| GlnPr | SEQ ID | CACCCCAGTTCCTATAAAT | forward | / |
| 1903 | No 37 | AGCAGAGCTCGTTTAGTGA | | |
| | | AC | | |
| GlnPr | SEQ ID | CGCTAGCACCGGTCGATCG | reverse | NheI |
| 1904 | No 38 | A | | |
| GlnPr | SEQ ID | TACTCGCGATTCACTGTAC | forward | NruI |
| 1905 | No 39 | AGTGGCACATAC | | |

1. An expression cassette which comprises a promoter, a polynucleotide sequence encoding a polypeptide, and expression enhancing element wherein expression enhancing element comprises a non-translated genomic DNA sequence downstream of a eukaryotic Glyceraldehyde 3-phosphate dehydrogenase (GAPDH) promoter, wherein the polypeptide encoded by the polynucleotide sequence is not GAPDH, and wherein the non-translated genomic DNA sequence downstream of the eukaryotic GAPDH promoter starts within a region spanning from nucleotide position around +1 to nucleotide position around +7000, wherein the nucleotide position is relative to the transcription start of the GAPDH mRNA, and wherein the length of the non-translated genomic DNA sequence downstream of the eukaryotic GAPDH promoter is from around 100 to around 15000 nucleotides.

2. The expression cassette of claim 1, wherein the expression cassette further comprises a non-translated genomic DNA sequence upstream of a eukaryotic GAPDH promoter, wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, and wherein the length of the non-translated genomic DNA sequence upstream of the eukaryotic GAPDH promoter is from around 100 to around 15000 nucleotides.

3. An expression cassette which comprises a promoter, a polynucleotide sequence encoding a polypeptide, and a non-translated genomic DNA sequence upstream of a eukaryotic GAPDH promoter, wherein the polypeptide encoded by the polynucleotide sequence is not GAPDH, and wherein the non-translated genomic DNA sequence upstream of the eukaryotic GAPDH promoter starts within a region spanning from around the 5′ end of the eukaryotic GAPDH promoter to nucleotide position around −3500, wherein the nucleotide position is relative to the transcription start of the GAPDH mRNA, wherein the length of the non-translated genomic DNA sequence upstream of the eukaryotic GAPDH promoter is from 100 to around 15000 nucleotides, with the proviso that the expression cassette does not comprise a eukaryotic GAPDH promoter or fragments thereof.

4. (canceled)

5. The expression cassette of claim 1, wherein the non-translated genomic DNA sequence downstream and/or upstream of the eukaryotic GAPDH promoter is not operably linked to the polynucleotide sequence encoding the polypeptide, and/or wherein the expression cassette further comprises a polyadenylation site.

6. (canceled)

7. The expression cassette of claim 1, wherein the length of the non-translated genomic DNA sequence downstream of the eukaryotic GAPDH promoter is around 10 nucleotides and extends at its maximum to the second to last intron of the IFF01 gene or to a part thereof, or wherein the non-translated genomic DNA sequence downstream of the eukaryotic GAPDH promoter starts downstream of the eukaryotic GAPDH polyadenylation site and wherein the length of the non-translated genomic DNA sequence downstream of the eukaryotic GAPDH promoter is at least around 100 nucleotides and extends at its maximum to the second to last intron of the IFF01 gene.

8. (canceled)

9. The expression cassette of claim 2, wherein the length of the non-translated genomic DNA sequence upstream of the eukaryotic GAPDH promoter is at least around 100 nucleotides and extends at its maximum to the start codon of the NCAPD2 gene, or wherein the length of the non-translated genomic DNA sequence upstream of the eukaryotic GAPDH promoter is at least around 100 nucleotides and extends at its maximum to the third to last intron of the NCAPD2 gene.

10. (canceled)

11. (canceled)

12. (canceled)

13. The expression cassette of claim 1, wherein the non-translated genomic DNA sequence downstream and/or upstream of the eukaryotic GAPDH promoter is of mammalian origin.

14. The expression cassette of claim 13, wherein the non-translated genomic DNA sequence downstream and/or upstream of the eukaryotic GAPDH promoter is of rodent or human origin.

15. The expression cassette of claim 1, wherein the non-translated genomic DNA sequence downstream of the eukaryotic GAPDH promoter comprises the nucleotide sequence selected from the group consisting of SEQ ID NOs: 8 and 21 or fragments thereof, or wherein the non-translated genomic DNA sequence downstream of the eukaryotic GAPDH promoter comprises a nucleotide sequence complementary to the nucleotide sequence selected from the group consisting of SEQ ID NOs: 8 and 21 or fragments thereof, or wherein the non-translated genomic DNA sequence downstream of the eukaryotic GAPDH promoter comprises a nucleotide sequence at least 80% identical to the nucleotide sequence selected from the group consisting of SEQ ID NOs: 8 and 21 or fragments thereof.

16. (canceled)

17. (canceled)

18. The expression cassette of claim 2, wherein the non-translated genomic DNA sequence upstream of the eukaryotic GAPDH promoter comprises a nucleotide sequence selected from the group consisting of SEQ ID NO: 7, 9, 10, 11, 12, 13, 14, 15, 16, 20, 22, 23, 24, 25, 26, 27 and 28 or fragments thereof, and/or wherein the nucleotide sequence selected from the group consisting of SEQ ID NOs: 7, 9, 10, 11, 12, 13, 14, 15, 16, 20, 22, 23, 24, 25, 26, 27 and 28 or fragments thereof comprises five or less nucleic acid modifications, or wherein the non-translated genomic DNA sequence upstream of the eukaryotic GAPDH promoter comprises a nucleotide sequence complementary to the nucleotide sequence selected from the group consisting of SEQ ID NO: 7, 9, 10, 11, 12, 13, 14, 15, 16, 20, 22, 23, 24, 25, 26, 27 and 28 or fragments thereof, or wherein the non-translated genomic DNA sequence upstream of the eukaryotic GAPDH promoter comprises a nucleotide sequence at least 80% identical to the nucleotide sequence selected from the group consisting of SEQ ID NO: 7, 9, 10, 11, 12, 13, 14, 15, 16, 20, 22, 23, 24, 25, 26, 27 and 28 or fragments thereof.

19. (canceled)

20. (canceled)

21. (canceled)

22. The expression cassette of claim 1, wherein the promoter and the polynucleotide sequence encoding a polypeptide are operatively linked.

23. The expression cassette of claim 1, wherein the non-translated genomic DNA sequence downstream of the eukaryotic GAPDH promoter is orientated in the same direction as the polynucleotide sequence encoding a polypeptide, or wherein the non-translated genomic DNA sequence downstream of the eukaryotic GAPDH promoter is oriented in opposite direction in relation to the polynucleotide sequence encoding a polvDeptide.

24. (canceled)

25. The expression cassette of claim 2, wherein the non-translated genomic DNA sequence upstream of the eukaryotic GAPDH promoter is oriented in the same direction as the polynucleotide sequence encoding a polypeptide, or wherein the non-translated genomic DNA sequence upstream of the eukaryotic GAPDH promoter is oriented in opposite direction in relation to the polynucleotide sequence encoding a polypeptide.

26. (canceled)

27. The expression cassette of claim 1, wherein the promoter is selected from the group consisting of SV40 promoter, MPSV promoter, mouse CMV, human tk, human CMV, rat CMV, human EF1alpha, Chinese hamster EF1alpha, human GAPDH, hybrid promoters including MYC, HYK and CX promoter, and/or wherein the polypeptide is selected from the group consisting of antibodies, antibody fragments and antibody derivatives and/or wherein the polyadenylation site is selected from the group consisting of BGH poly(A) and SV40 poly(A).

28. (canceled)

29. (canceled)

30. The expression cassette of claim 1, further comprising a genetic element selected from the group consisting of an additional promoter, an enhancer, transcriptional control elements, and a selectable marker.

31. The expression cassette of claim 30, wherein the genetic element is a selectable marker wherein the content of CpG sites contained in the polynucleotide sequence encoding the selectable marker is 45 or less.

32. An expression vector comprising the expression cassette of claim 1.

33. (canceled)

34. (canceled)

35. (canceled)

36. A host cell comprising the vector comprising the expression cassette of claim 32.

37. The expression cassette of claim 1 for use as a medicament for the treatment of a disorder, and/or for use in gene therapy.

38. (canceled)

39. An in vitro method for the expression of a polypeptide, comprising transfecting a host cell with the expression cassette of claim 1 and recovering the polypeptide.

40. (canceled)

41. (canceled)

42. Use of the expression cassette of claim 1 for the expression of a heterologous polypeptide from a mammalian host cell.
